SEQUENCE LISTING (304 sequences)

SEQ ID NO: 1 - protein, 5 aa, Artificial sequence
  synthetic
SFEVH

SEQ ID NO: 2 - protein, 5 aa, Artificial sequence
  synthetic
FEVHV

SEQ ID NO: 3 - protein, 5 aa, Artificial sequence
  synthetic
EVHVC

SEQ ID NO: 4 - protein, 5 aa, Artificial sequence
  synthetic
VHVCA

SEQ ID NO: 5 - protein, 5 aa, Artificial sequence
  synthetic
HVCAC

SEQ ID NO: 6 - protein, 5 aa, Artificial sequence
  synthetic
PSTGR

SEQ ID NO: 7 - protein, 5 aa, Artificial sequence
  synthetic
STGRV

SEQ ID NO: 8 - protein, 5 aa, Artificial sequence
  synthetic
TGRVK

SEQ ID NO: 9 - protein, 5 aa, Artificial sequence
  synthetic
GRVKK

SEQ ID NO: 10 - protein, 5 aa, Artificial sequence
  synthetic
RVKKP

SEQ ID NO: 11 - protein, 5 aa, Artificial sequence
  synthetic
KLGTA

SEQ ID NO: 12 - protein, 5 aa, Artificial sequence
  synthetic
LGTAV

SEQ ID NO: 13 - protein, 5 aa, Artificial sequence
  synthetic
GTAVK

SEQ ID NO: 14 - protein, 5 aa, Artificial sequence
  synthetic
TAVKP

SEQ ID NO: 15 - protein, 5 aa, Artificial sequence
  synthetic
AVKPK

SEQ ID NO: 16 - protein, 5 aa, Artificial sequence
  synthetic
RNFVH

SEQ ID NO: 17 - protein, 5 aa, Artificial sequence
  synthetic
NSEHV

SEQ ID NO: 18 - protein, 5 aa, Artificial sequence
  synthetic
FEHCA

SEQ ID NO: 19 - protein, 5 aa, Artificial sequence
  synthetic
VHCCP

SEQ ID NO: 20 - protein, 5 aa, Artificial sequence
  synthetic
HVAPG

SEQ ID NO: 21 - protein, 5 aa, Artificial sequence
  synthetic
SASGR

SEQ ID NO: 22 - protein, 5 aa, Artificial sequence
  synthetic
APTRV

SEQ ID NO: 23 - protein, 5 aa, Artificial sequence
  synthetic
STRKK

SEQ ID NO: 24 - protein, 5 aa, Artificial sequence
  synthetic
GRKPH

SEQ ID NO: 25 - protein, 5 aa, Artificial sequence
  synthetic
RVKHR

SEQ ID NO: 26 - protein, 5 aa, Artificial sequence
  synthetic
VKPRY

SEQ ID NO: 27 - protein, 5 aa, Artificial sequence
  synthetic
KKHYR

SEQ ID NO: 28 - protein, 5 aa, Artificial sequence
  synthetic
SGLTA

SEQ ID NO: 29 - protein, 5 aa, Artificial sequence
  synthetic
GKGAV

SEQ ID NO: 30 - protein, 5 aa, Artificial sequence
  synthetic
LGAKP

SEQ ID NO: 31 - protein, 5 aa, Artificial sequence
  synthetic
TAKKS

SEQ ID NO: 32 - protein, 5 aa, Artificial sequence
  synthetic
AVPSL

SEQ ID NO: 33 - protein, 9 aa, Artificial sequence
  synthetic
SNFEVHVCK

SEQ ID NO: 34 - protein, 9 aa, Artificial sequence
  synthetic
SGNVHVCAF

SEQ ID NO: 35 - protein, 9 aa, Artificial sequence
  synthetic
FEVHVCACP

SEQ ID NO: 36 - protein, 9 aa, Artificial sequence
  synthetic
GSGHVCACR

SEQ ID NO: 37 - protein, 9 aa, Artificial sequence
  synthetic
HAASTGRVK

SEQ ID NO: 38 - protein, 9 aa, Artificial sequence
  synthetic
PGWSTGRVP

SEQ ID NO: 39 - protein, 9 aa, Artificial sequence
  synthetic
SSWTGRVKP

SEQ ID NO: 40 - protein, 9 aa, Artificial sequence
  synthetic
FSPRVKKPK

SEQ ID NO: 41 - protein, 9 aa, Artificial sequence
  synthetic
PFKLGTAVK

SEQ ID NO: 42 - protein, 9 aa, Artificial sequence
  synthetic
SGKLGTAVK

SEQ ID NO: 43 - protein, 9 aa, Artificial sequence
  synthetic
SASGTAVKP

SEQ ID NO: 44 - protein, 9 aa, Artificial sequence
  synthetic
SPQTAVKPK

SEQ ID NO: 45 - protein, 15 aa, Artificial sequence
  synthetic
PLGKRNSFEVHWWAL

SEQ ID NO: 46 - protein, 15 aa, Artificial sequence
  synthetic
PALHRNSFEVHWWNN

SEQ ID NO: 47 - protein, 15 aa, Artificial sequence
  synthetic
VLKHNSFEVHVNMLA

SEQ ID NO: 48 - protein, 15 aa, Artificial sequence
  synthetic
NGLWSTGRVKKWLLG

SEQ ID NO: 49 - protein, 15 aa, Artificial sequence
  synthetic
NWLMSTGRVKKLHVL

SEQ ID NO: 50 - protein, 15 aa, Artificial sequence
  synthetic
NLGHGRVKKPHWLLL

SEQ ID NO: 51 - protein, 15 aa, Artificial sequence
  synthetic
WGSWSGKLGTASHNL

SEQ ID NO: 52 - protein, 15 aa, Artificial sequence
  synthetic
HANLSGKLGTAAMLS

SEQ ID NO: 53 - protein, 15 aa, Artificial sequence
  synthetic
WPWALGTAVKPKKLA

SEQ ID NO: 54 - protein, 5 aa, Artificial sequence
  synthetic
GGMNQ

SEQ ID NO: 55 - protein, 5 aa, Artificial sequence
  synthetic
GMNQR

SEQ ID NO: 56 - protein, 5 aa, Artificial sequence
  synthetic
MNQRP

SEQ ID NO: 57 - protein, 5 aa, Artificial sequence
  synthetic
NQRPI

SEQ ID NO: 58 - protein, 5 aa, Artificial sequence
  synthetic
QRPIL

SEQ ID NO: 59 - protein, 5 aa, Artificial sequence
  synthetic
YTEFI

SEQ ID NO: 60 - protein, 5 aa, Artificial sequence
  synthetic
TEFIT

SEQ ID NO: 61 - protein, 5 aa, Artificial sequence
  synthetic
EFITP

SEQ ID NO: 62 - protein, 5 aa, Artificial sequence
  synthetic
FITPD

SEQ ID NO: 63 - protein, 5 aa, Artificial sequence
  synthetic
ITPDG

SEQ ID NO: 64 - protein, 5 aa, Artificial sequence
  synthetic
TSQKL

SEQ ID NO: 65 - protein, 5 aa, Artificial sequence
  synthetic
SQKLI

SEQ ID NO: 66 - protein, 5 aa, Artificial sequence
  synthetic
QKLIL

SEQ ID NO: 67 - protein, 5 aa, Artificial sequence
  synthetic
KLILE

SEQ ID NO: 68 - protein, 5 aa, Artificial sequence
  synthetic
LILEL

SEQ ID NO: 69 - protein, 5 aa, Artificial sequence
  synthetic
YTGWG

SEQ ID NO: 70 - protein, 5 aa, Artificial sequence
  synthetic
TGWGN

SEQ ID NO: 71 - protein, 5 aa, Artificial sequence
  synthetic
GWGNV

SEQ ID NO: 72 - protein, 5 aa, Artificial sequence
  synthetic
WGNVK

SEQ ID NO: 73 - protein, 5 aa, Artificial sequence
  synthetic
GNVKS

SEQ ID NO: 74 - protein, 5 aa, Artificial sequence
  synthetic
PMGSK

SEQ ID NO: 75 - protein, 5 aa, Artificial sequence
  synthetic
MGSKP

SEQ ID NO: 76 - protein, 5 aa, Artificial sequence
  synthetic
GSKPV

SEQ ID NO: 77 - protein, 5 aa, Artificial sequence
  synthetic
SKPVP

SEQ ID NO: 78 - protein, 5 aa, Artificial sequence
  synthetic
KPVPK

SEQ ID NO: 79 - protein, 5 aa, Artificial sequence
  synthetic
LEKFW

SEQ ID NO: 80 - protein, 5 aa, Artificial sequence
  synthetic
EKFWD

SEQ ID NO: 81 - protein, 5 aa, Artificial sequence
  synthetic
KFWDN

SEQ ID NO: 82 - protein, 5 aa, Artificial sequence
  synthetic
FWDNC

SEQ ID NO: 83 - protein, 5 aa, Artificial sequence
  synthetic
WDNCL

SEQ ID NO: 84 - protein, 5 aa, Artificial sequence
  synthetic
CMGNQ

SEQ ID NO: 85 - protein, 5 aa, Artificial sequence
  synthetic
MGMQR

SEQ ID NO: 86 - protein, 5 aa, Artificial sequence
  synthetic
GMQPI

SEQ ID NO: 87 - protein, 5 aa, Artificial sequence
  synthetic
MNRIL

SEQ ID NO: 88 - protein, 5 aa, Artificial sequence
  synthetic
NQPLT

SEQ ID NO: 89 - protein, 5 aa, Artificial sequence
  synthetic
QRITI

SEQ ID NO: 90 - protein, 5 aa, Artificial sequence
  synthetic
RPLII

SEQ ID NO: 91 - protein, 5 aa, Artificial sequence
  synthetic
FNTFI

SEQ ID NO: 92 - protein, 5 aa, Artificial sequence
  synthetic
NYEIT

SEQ ID NO: 93 - protein, 5 aa, Artificial sequence
  synthetic
TEIPD

SEQ ID NO: 94 - protein, 5 aa, Artificial sequence
  synthetic
EFTDG

SEQ ID NO: 95 - protein, 5 aa, Artificial sequence
  synthetic
FIPGH

SEQ ID NO: 96 - protein, 5 aa, Artificial sequence
  synthetic
ITDHY

SEQ ID NO: 97 - protein, 5 aa, Artificial sequence
  synthetic
TPGYY

SEQ ID NO: 98 - protein, 5 aa, Artificial sequence
  synthetic
LDSKL

SEQ ID NO: 99 - protein, 5 aa, Artificial sequence
  synthetic
DTQLI

SEQ ID NO: 100 - protein, 5 aa, Artificial sequence
  synthetic
SQLLE

SEQ ID NO: 101 - protein, 5 aa, Artificial sequence
  synthetic
QKIEL

SEQ ID NO: 102 - protein, 5 aa, Artificial sequence
  synthetic
KLLLE

SEQ ID NO: 103 - protein, 5 aa, Artificial sequence
  synthetic
LIEES

SEQ ID NO: 104 - protein, 5 aa, Artificial sequence
  synthetic
ILLSH

SEQ ID NO: 105 - protein, 5 aa, Artificial sequence
  synthetic
VYTWG

SEQ ID NO: 106 - protein, 5 aa, Artificial sequence
  synthetic
YYGGN

SEQ ID NO: 107 - protein, 5 aa, Artificial sequence
  synthetic
TGGVK

SEQ ID NO: 108 - protein, 5 aa, Artificial sequence
  synthetic
GWNKS

SEQ ID NO: 109 - protein, 5 aa, Artificial sequence
  synthetic
WGVSF

SEQ ID NO: 110 - protein, 5 aa, Artificial sequence
  synthetic
GNKFL

SEQ ID NO: 111 - protein, 5 aa, Artificial sequence
  synthetic
NVSLT

SEQ ID NO: 112 - protein, 5 aa, Artificial sequence
  synthetic
VKMSK

SEQ ID NO: 113 - protein, 5 aa, Artificial sequence
  synthetic
KPGKP

SEQ ID NO: 114 - protein, 5 aa, Artificial sequence
  synthetic
MGKVP

SEQ ID NO: 115 - protein, 5 aa, Artificial sequence
  synthetic
GSPPK

SEQ ID NO: 116 - protein, 5 aa, Artificial sequence
  synthetic
SKVKS

SEQ ID NO: 117 - protein, 5 aa, Artificial sequence
  synthetic
KPPSR

SEQ ID NO: 118 - protein, 5 aa, Artificial sequence
  synthetic
PVKRI

SEQ ID NO: 119 - protein, 5 aa, Artificial sequence
  synthetic
ELEFW

SEQ ID NO: 120 - protein, 5 aa, Artificial sequence
  synthetic
LLKWD

SEQ ID NO: 121 - protein, 5 aa, Artificial sequence
  synthetic
EKWNC

SEQ ID NO: 122 - protein, 5 aa, Artificial sequence
  synthetic
KFDCL

SEQ ID NO: 123 - protein, 5 aa, Artificial sequence
  synthetic
FWNLA

SEQ ID NO: 124 - protein, 5 aa, Artificial sequence
  synthetic
WDCAI

SEQ ID NO: 125 - protein, 5 aa, Artificial sequence
  synthetic
DNLIL

SEQ ID NO: 126 - protein, 9 aa, Artificial sequence
  synthetic
GVAEKFWDL

SEQ ID NO: 127 - protein, 9 aa, Artificial sequence
  synthetic
LLEKFWDNC

SEQ ID NO: 128 - protein, 9 aa, Artificial sequence
  synthetic
SPVWDNCLT

SEQ ID NO: 129 - protein, 9 aa, Artificial sequence
  synthetic
ETTYTGWGY

SEQ ID NO: 130 - protein, 9 aa, Artificial sequence
  synthetic
KLPTGWGNF

SEQ ID NO: 131 - protein, 9 aa, Artificial sequence
  synthetic
HPIWGNVKY

SEQ ID NO: 132 - protein, 9 aa, Artificial sequence
  synthetic
VANTEFITV

SEQ ID NO: 133 - protein, 9 aa, Artificial sequence
  synthetic
AGDFITPDA

SEQ ID NO: 134 - protein, 9 aa, Artificial sequence
  synthetic
WPQFITPDV

SEQ ID NO: 135 - protein, 9 aa, Artificial sequence
  synthetic
GVKPMGSKP

SEQ ID NO: 136 - protein, 9 aa, Artificial sequence
  synthetic
PPGGSKPVG

SEQ ID NO: 137 - protein, 9 aa, Artificial sequence
  synthetic
SGSKPVPKY

SEQ ID NO: 138 - protein, 9 aa, Artificial sequence
  synthetic
TLLGMNQRS

SEQ ID NO: 139 - protein, 9 aa, Artificial sequence
  synthetic
AGEMNQRPI

SEQ ID NO: 140 - protein, 9 aa, Artificial sequence
  synthetic
MGGMNQRPI

SEQ ID NO: 141 - protein, 9 aa, Artificial sequence
  synthetic
GMNQRPILT

SEQ ID NO: 142 - protein, 9 aa, Artificial sequence
  synthetic
FSLTSQKLL

SEQ ID NO: 143 - protein, 9 aa, Artificial sequence
  synthetic
LPSSQKLIL

SEQ ID NO: 144 - protein, 9 aa, Artificial sequence
  synthetic
LGKKLILEP

SEQ ID NO: 145 - protein, 15 aa, Artificial sequence
  synthetic
SVVIEKFWDNCASYV

SEQ ID NO: 146 - protein, 15 aa, Artificial sequence
  synthetic
LLEKFWDNCLAILES

SEQ ID NO: 147 - protein, 15 aa, Artificial sequence
  synthetic
LEKFWDNCLAILESK

SEQ ID NO: 148 - protein, 15 aa, Artificial sequence
  synthetic
DPVYTEFITPDWLRY

SEQ ID NO: 149 - protein, 15 aa, Artificial sequence
  synthetic
RAQYTEFITPDIPPR

SEQ ID NO: 150 - protein, 15 aa, Artificial sequence
  synthetic
FTQWITPDGHYKVKL

SEQ ID NO: 151 - protein, 15 aa, Artificial sequence
  synthetic
SSLFMGGMNQRQIWR

SEQ ID NO: 152 - protein, 15 aa, Artificial sequence
  synthetic
QVVFGMNQRPIPWPP

SEQ ID NO: 153 - protein, 15 aa, Artificial sequence
  synthetic
PFAFNQRPILTLPKE

SEQ ID NO: 154 - protein, 15 aa, Artificial sequence
  synthetic
VILTVYYTGWGWRSR

SEQ ID NO: 155 - protein, 15 aa, Artificial sequence
  synthetic
SILLVYYTGWGWRDS

SEQ ID NO: 156 - protein, 15 aa, Artificial sequence
  synthetic
RLQLWGNVKSFGFQQ

SEQ ID NO: 157 - protein, 15 aa, Artificial sequence
  synthetic
KGFYMGSKPVPPRRR

SEQ ID NO: 158 - protein, 15 aa, Artificial sequence
  synthetic
RQISKLILELEVYYP

SEQ ID NO: 159 - protein, 15 aa, Artificial sequence
  synthetic
GLWELILELESNFQA

SEQ ID NO: 160 - protein, 15 aa, Artificial sequence
  synthetic
SVVIEKFWDNCASYV

SEQ ID NO: 161 - protein, 15 aa, Artificial sequence
  synthetic
LLEKFWDNCLAILES

SEQ ID NO: 162 - skipped

SEQ ID NO: 163 - protein, 5 aa, Artificial sequence
  synthetic
NPCSD

SEQ ID NO: 164 - protein, 5 aa, Artificial sequence
  synthetic
PCSDL

SEQ ID NO: 165 - protein, 5 aa, Artificial sequence
  synthetic
CSDLI

SEQ ID NO: 166 - protein, 5 aa, Artificial sequence
  synthetic
SDLIR

SEQ ID NO: 167 - protein, 5 aa, Artificial sequence
  synthetic
SKTLG

SEQ ID NO: 168 - protein, 5 aa, Artificial sequence
  synthetic
PCSKT

SEQ ID NO: 169 - protein, 5 aa, Artificial sequence
  synthetic
CSKTL

SEQ ID NO: 170 - protein, 5 aa, Artificial sequence
  synthetic
IGCPD

SEQ ID NO: 171 - protein, 5 aa, Artificial sequence
  synthetic
GCPDL

SEQ ID NO: 172 - protein, 5 aa, Artificial sequence
  synthetic
CPDLI

SEQ ID NO: 173 - protein, 5 aa, Artificial sequence
  synthetic
PDLIR

SEQ ID NO: 174 - protein, 9 aa, Artificial sequence
  synthetic
TANNPCSDL

SEQ ID NO: 175 - protein, 9 aa, Artificial sequence
  synthetic
ANNPCSDLI

SEQ ID NO: 176 - protein, 9 aa, Artificial sequence
  synthetic
NNPCSDLIR

SEQ ID NO: 177 - protein, 9 aa, Artificial sequence
  synthetic
NPCSDLIRD

SEQ ID NO: 178 - protein, 9 aa, Artificial sequence
  synthetic
NPCSKTLGR

SEQ ID NO: 179 - protein, 9 aa, Artificial sequence
  synthetic
ANNPCSKTL

SEQ ID NO: 180 - protein, 9 aa, Artificial sequence
  synthetic
NNPCSKTLG

SEQ ID NO: 181 - protein, 9 aa, Artificial sequence
  synthetic
SAYIGCPDL

SEQ ID NO: 182 - protein, 9 aa, Artificial sequence
  synthetic
AYIGCPDLI

SEQ ID NO: 183 - protein, 9 aa, Artificial sequence
  synthetic
YIGCPDLIR

SEQ ID NO: 184 - protein, 9 aa, Artificial sequence
  synthetic
IGCPDLIRD

SEQ ID NO: 185 - protein, 9 aa, Artificial sequence
  synthetic
GLPNPCSDL

SEQ ID NO: 186 - protein, 9 aa, Artificial sequence
  synthetic
FAVNPCSDV

SEQ ID NO: 187 - protein, 9 aa, Artificial sequence
  synthetic
NIYPCSDLV

SEQ ID NO: 188 - protein, 9 aa, Artificial sequence
  synthetic
VMQPCSDLL

SEQ ID NO: 189 - protein, 9 aa, Artificial sequence
  synthetic
IDTCSDLIK

SEQ ID NO: 190 - protein, 9 aa, Artificial sequence
  synthetic
EVRCSDLIP

SEQ ID NO: 191 - protein, 9 aa, Artificial sequence
  synthetic
RAECSDLIP

SEQ ID NO: 192 - protein, 9 aa, Artificial sequence
  synthetic
GSNCSDLIP

SEQ ID NO: 193 - protein, 9 aa, Artificial sequence
  synthetic
IVASDLIRY

SEQ ID NO: 194 - protein, 9 aa, Artificial sequence
  synthetic
SIQSDLIRP

SEQ ID NO: 195 - protein, 9 aa, Artificial sequence
  synthetic
EPRCSDLIR

SEQ ID NO: 196 - protein, 9 aa, Artificial sequence
  synthetic
RAVCSDLIK

SEQ ID NO: 197 - protein, 9 aa, Artificial sequence
  synthetic
VGTSDLIRK

SEQ ID NO: 198 - protein, 9 aa, Artificial sequence
  synthetic
TVLSDLIRK

SEQ ID NO: 199 - protein, 9 aa, Artificial sequence
  synthetic
APPNPCSDW

SEQ ID NO: 200 - protein, 9 aa, Artificial sequence
  synthetic
NPLNPCSDA

SEQ ID NO: 201 - protein, 9 aa, Artificial sequence
  synthetic
PGRPCSDLT

SEQ ID NO: 202 - protein, 9 aa, Artificial sequence
  synthetic
KGEPCSDLL

SEQ ID NO: 203 - protein, 9 aa, Artificial sequence
  synthetic
KPMSDLIRG

SEQ ID NO: 204 - protein, 9 aa, Artificial sequence
  synthetic
KPESDLIRA

SEQ ID NO: 205 - protein, 9 aa, Artificial sequence
  synthetic
GARNPCSDV

SEQ ID NO: 206 - protein, 9 aa, Artificial sequence
  synthetic
AARNPCSDV

SEQ ID NO: 207 - protein, 9 aa, Artificial sequence
  synthetic
YSRPCSDLV

SEQ ID NO: 208 - protein, 9 aa, Artificial sequence
  synthetic
RTRPCSDLV

SEQ ID NO: 209 - protein, 9 aa, Artificial sequence
  synthetic
YTFCSDLIT

SEQ ID NO: 210 - protein, 9 aa, Artificial sequence
  synthetic
RNECSDLIR

SEQ ID NO: 211 - protein, 9 aa, Artificial sequence
  synthetic
LGKSDLIRF

SEQ ID NO: 212 - protein, 9 aa, Artificial sequence
  synthetic
RVVSDLIRF

SEQ ID NO: 213 - protein, 9 aa, Artificial sequence
  synthetic
LVWSKTLGD

SEQ ID NO: 214 - protein, 9 aa, Artificial sequence
  synthetic
IPLSKTLGR

SEQ ID NO: 215 - protein, 9 aa, Artificial sequence
  synthetic
DAFSKTLGR

SEQ ID NO: 216 - protein, 9 aa, Artificial sequence
  synthetic
ASGSKTLGR

SEQ ID NO: 217 - protein, 9 aa, Artificial sequence
  synthetic
FSVSKTLGK

SEQ ID NO: 218 - protein, 9 aa, Artificial sequence
  synthetic
VAPSKTLGR

SEQ ID NO: 219 - protein, 9 aa, Artificial sequence
  synthetic
FMRPCSKTI

SEQ ID NO: 220 - protein, 9 aa, Artificial sequence
  synthetic
LPIPCSKTP

SEQ ID NO: 221 - protein, 9 aa, Artificial sequence
  synthetic
LASSKTLGL

SEQ ID NO: 222 - protein, 9 aa, Artificial sequence
  synthetic
AGMSKTLGL

SEQ ID NO: 223 - protein, 9 aa, Artificial sequence
  synthetic
VSKPCSKTV

SEQ ID NO: 224 - protein, 9 aa, Artificial sequence
  synthetic
KLRPCSKTL

SEQ ID NO: 225 - protein, 9 aa, Artificial sequence
  synthetic
ARSCSKTLS

SEQ ID NO: 226 - protein, 9 aa, Artificial sequence
  synthetic
PQLCSKTLL

SEQ ID NO: 227 - protein, 9 aa, Artificial sequence
  synthetic
LKDSKTLGS

SEQ ID NO: 228 - protein, 9 aa, Artificial sequence
  synthetic
HNFSKTLGR

SEQ ID NO: 229 - protein, 9 aa, Artificial sequence
  synthetic
VITSKTLGY

SEQ ID NO: 230 - protein, 9 aa, Artificial sequence
  synthetic
FKFSKTLGY

SEQ ID NO: 231 - protein, 9 aa, Artificial sequence
  synthetic
HMAIGCPDL

SEQ ID NO: 232 - protein, 9 aa, Artificial sequence
  synthetic
KIAIGCPDV

SEQ ID NO: 233 - protein, 9 aa, Artificial sequence
  synthetic
RVEGCPDLV

SEQ ID NO: 234 - protein, 9 aa, Artificial sequence
  synthetic
YLEGCPDLS

SEQ ID NO: 235 - protein, 9 aa, Artificial sequence
  synthetic
TSLCPDLIP

SEQ ID NO: 236 - protein, 9 aa, Artificial sequence
  synthetic
QAQCPDLIP

SEQ ID NO: 237 - protein, 9 aa, Artificial sequence
  synthetic
KTSPDLIRY

SEQ ID NO: 238 - protein, 9 aa, Artificial sequence
  synthetic
VSAPDLIRP

SEQ ID NO: 239 - protein, 9 aa, Artificial sequence
  synthetic
AVVIGCPDP

SEQ ID NO: 240 - protein, 9 aa, Artificial sequence
  synthetic
QNHIGCPDK

SEQ ID NO: 241 - protein, 9 aa, Artificial sequence
  synthetic
PEQCPDLIK

SEQ ID NO: 242 - protein, 9 aa, Artificial sequence
  synthetic
WSACPDLIP

SEQ ID NO: 243 - protein, 9 aa, Artificial sequence
  synthetic
AQSPDLIRR

SEQ ID NO: 244 - protein, 9 aa, Artificial sequence
  synthetic
SEYPDLIRR

SEQ ID NO: 245 - protein, 9 aa, Artificial sequence
  synthetic
KAVCPDLIR

SEQ ID NO: 246 - protein, 9 aa, Artificial sequence
  synthetic
QPTCPDLIR

SEQ ID NO: 247 - protein, 9 aa, Artificial sequence
  synthetic
RPPPDLIRR

SEQ ID NO: 248 - protein, 9 aa, Artificial sequence
  synthetic
SSLPDLIRK

SEQ ID NO: 249 - protein, 9 aa, Artificial sequence
  synthetic
SPLIGCPDY

SEQ ID NO: 250 - protein, 9 aa, Artificial sequence
  synthetic
RGRIGCPDF

SEQ ID NO: 251 - protein, 9 aa, Artificial sequence
  synthetic
HLTGCPDLV

SEQ ID NO: 252 - protein, 9 aa, Artificial sequence
  synthetic
EAKGCPDLV

SEQ ID NO: 253 - protein, 9 aa, Artificial sequence
  synthetic
TRRGCPDLL

SEQ ID NO: 254 - protein, 9 aa, Artificial sequence
  synthetic
YKSGCPDLP

SEQ ID NO: 255 - protein, 9 aa, Artificial sequence
  synthetic
YVPIGCPDW

SEQ ID NO: 256 - protein, 9 aa, Artificial sequence
  synthetic
QATIGCPDW

SEQ ID NO: 257 - protein, 9 aa, Artificial sequence
  synthetic
NAKGCPDLW

SEQ ID NO: 258 - protein, 9 aa, Artificial sequence
  synthetic
RLVGCPDLF

SEQ ID NO: 259 - protein, 9 aa, Artificial sequence
  synthetic
VPVCPDLIR

SEQ ID NO: 260 - protein, 9 aa, Artificial sequence
  synthetic
KLHCPDLIF

SEQ ID NO: 261 - protein, 9 aa, Artificial sequence
  synthetic
LPQPDLIRF

SEQ ID NO: 262 - protein, 9 aa, Artificial sequence
  synthetic
VASPDLIRF

SEQ ID NO: 263 - protein, 5 aa, Artificial sequence
  synthetic
PCDIR

SEQ ID NO: 264 - protein, 5 aa, Artificial sequence
  synthetic
SDIDQ

SEQ ID NO: 265 - protein, 5 aa, Artificial sequence
  synthetic
KTGRD

SEQ ID NO: 266 - protein, 5 aa, Artificial sequence
  synthetic
GCDIR

SEQ ID NO: 267 - protein, 5 aa, Artificial sequence
  synthetic
CPLRD

SEQ ID NO: 268 - protein, 5 aa, Artificial sequence
  synthetic
PDIDQ

SEQ ID NO: 269 - protein, 15 aa, Artificial sequence
  synthetic
TANNPCSDLIRDQGF

SEQ ID NO: 270 - protein, 15 aa, Artificial sequence
  synthetic
NNPCSDLIRDQGFRG

SEQ ID NO: 271 - protein, 15 aa, Artificial sequence
  synthetic
NPCSKTLGRRDSSDD

SEQ ID NO: 272 - protein, 15 aa, Artificial sequence
  synthetic
SAYIGCPDLIRDQGF

SEQ ID NO: 273 - protein, 15 aa, Artificial sequence
  synthetic
AYIGCPDLIRDQGFR

SEQ ID NO: 274 - protein, 15 aa, Artificial sequence
  synthetic
YIGCPDLIRDQGFRG

SEQ ID NO: 275 - protein, 15 aa, Artificial sequence
  synthetic
IAFTPCSDLIRRTFS

SEQ ID NO: 276 - protein, 15 aa, Artificial sequence
  synthetic
FGRLPCSDLIRVFDK

SEQ ID NO: 277 - protein, 15 aa, Artificial sequence
  synthetic
RPRLPCSDLIRVPHP

SEQ ID NO: 278 - protein, 15 aa, Artificial sequence
  synthetic
PSILPCSDLIRSTKL

SEQ ID NO: 279 - protein, 15 aa, Artificial sequence
  synthetic
RSIISDLIRDQGFVA

SEQ ID NO: 280 - protein, 15 aa, Artificial sequence
  synthetic
GSFLSDLIRDQMPPE

SEQ ID NO: 281 - protein, 15 aa, Artificial sequence
  synthetic
TVLVKTLGRRDLWQL

SEQ ID NO: 282 - protein, 15 aa, Artificial sequence
  synthetic
IAAFKTLGRRDKNRE

SEQ ID NO: 283 - protein, 15 aa, Artificial sequence
  synthetic
ISYFGCPDLIRASLM

SEQ ID NO: 284 - protein, 15 aa, Artificial sequence
  synthetic
TRFLGCPDLIRGSLM

SEQ ID NO: 285 - protein, 15 aa, Artificial sequence
  synthetic
HLFICPDLIRDSLLH

SEQ ID NO: 286 - protein, 15 aa, Artificial sequence
  synthetic
AFSWCPDLIRDVSYH

SEQ ID NO: 287 - protein, 15 aa, Artificial sequence
  synthetic
DIYLPDLIRDQVGTS

SEQ ID NO: 288 - protein, 15 aa, Artificial sequence
  synthetic
IPVLPDLIRDQFSLS

SEQ ID NO: 289 - protein, 15 aa, Artificial sequence
  synthetic
LDMVGCPDLIRRVGV

SEQ ID NO: 290 - protein, 15 aa, Artificial sequence
  synthetic
VHEQGCPDLIRWFLH

SEQ ID NO: 291 - protein, 15 aa, Artificial sequence
  synthetic
SDLICPDLIRDPQQL

SEQ ID NO: 292 - protein, 15 aa, Artificial sequence
  synthetic
VELLCPDLIRDLNNQ

SEQ ID NO: 293 - protein, 15 aa, Artificial sequence
  synthetic
YLILPDLIRDQEQTW

SEQ ID NO: 294 - protein, 15 aa, Artificial sequence
  synthetic
VYLRPDLIRDQQKFA

SEQ ID NO: 295 - protein, 15 aa, Artificial sequence
  synthetic
YTWRGCPDLIRPTIF

SEQ ID NO: 296 - protein, 15 aa, Artificial sequence
  synthetic
YVVYGCPDLIRPQIV

SEQ ID NO: 297 - protein, 15 aa, Artificial sequence
  synthetic
TTVLPDLIRDQEAAR

SEQ ID NO: 298 - protein, 15 aa, Artificial sequence
  synthetic
ARLEPDLIRDQLLSS

SEQ ID NO: 299 - protein, 15 aa, Artificial sequence
  synthetic
QIQVGCPDLIRVLPL

SEQ ID NO: 300 - protein, 15 aa, Artificial sequence
  synthetic
TVLFGCPDLIRWNRT

SEQ ID NO: 301 - protein, 15 aa, Artificial sequence
  synthetic
FLKLCPDLIRDPAYP

SEQ ID NO: 302 - protein, 15 aa, Artificial sequence
  synthetic
HPVPCPDLIRDPLWT

SEQ ID NO: 303 - protein, 15 aa, Artificial sequence
  synthetic
RLLHPDLIRDQIVVG

SEQ ID NO: 304 - protein, 15 aa, Artificial sequence
  synthetic
APEFPDLIRDQYYLA

(19) United States
(12) Patent Application Publication
(10) Pub. No.: US 2024/0197878 A1
(43) Pub. Date: Jun. 20, 2024

(54) PERSONALIZED ALLOGENEIC IMMUNOTHERAPY

(71) Applicant: IOGENETICS, LLC, Sun Prairie, WI (US)

(72) Inventors: Jane Homan, Marshall, WI (US); Robert D. Bremel, Marshall, WI (US)

(21) Appl. No.: 18/287,715

(22) PCT Filed: Apr. 20, 2022
(86) PCT No.: PCT/US2022/025527

§ 371 Date: Oct. 20, 2023

Related U.S. Application Data
(60) Provisional application No. 63/178,147, filed on Apr. 22, 2021.

(51) Int. Cl. A61K 39/00; A61P 35/00; C12N 15/10; G16B 15/30; G16B 20/20

(52) U.S. Cl. CPC A61K 39/464401; A61K 39/4611; A61P 35/00; C12N 15/1055; G16B 15/30; G16B 20/20

(57) ABSTRACT

The present invention provides methods for treating cancer by T cell therapy comprising the steps of obtaining a biopsy from a subject affected by cancer, identifying mutated amino acids in the tumor and the T cell exposed amino acid motifs which contain the mutated amino acids, identifying a donor with matching alleles, generating an array of alternate peptides in which the T cell exposed motifs are maintained constant, but the other amino acids are substituted, selecting one or more peptides from the array of alternative peptides, each having a desired binding affinity to the MHC allele while maintaining the tumor specific T cell exposed motif, contacting antigen presenting cells with the selected alternative peptides so that the peptide is presented by the MHC of the antigen presenting cells, contacting the antigen presenting cells carrying the selected peptide with T cells harvested from the donor, and infusing the subject with stimulated T cells responding to the peptide of interest presented by the dendritic cell MHC.

### CROSS-REFERENCE TO RELATED APPLICATION

The present application claims priority to and the benefit of U.S. Provisional Patent Application No. 63/178,147, filed Apr. 22, 2021. The contents of which are hereby incorporated by reference in its entirety.

### FIELD OF THE INVENTION

The present invention relates to methods for the selection and expansion of T cell clones from an MHC allele-matched donor for transfer into a subject affected by cancer, wherein the donor T cells are stimulated ex vivo to respond to a selected T cell exposed motif in a target peptide, wherein the T cell exposed motif comprises an amino acid of interest, and in particular the T cell exposed motif comprises an amino acid mutated in the tumor, and the selected peptide has been selected to bind preferentially to the shared MHC alleles of donor and subject.

### BACKGROUND OF THE INVENTION

Cancer is a leading cause of death of children over one year of age in developed countries [41]. The spectrum of pediatric cancers is significantly different from that seen in adults. Pediatric cancers may arise from mutations which occur relatively early in embryonic development, and throughout fetal life and early childhood. The most common brain and solid tumors that arise in children, including medulloblastoma, neuroblastoma, rhabdomyosarcoma. Ewing sarcoma, osteosarcoma and Wilms' tumor, are exceedingly rare in adults [42]. This difference is not unexpected, in that many pediatric cancers are thought to arise within developing tissues as they undergo substantial expansion during early organ formation, growth and maturation. Mutational frequencies are many times lower overall in childhood cancers than in their adult counterparts [43]. Only those tumors arising from mutations in mismatch repair gene mutations have overall higher mutational frequencies more comparable to those seen in adult cancers. As with adult cancers, mutations include missense mutations, insertions and deletions, with and without frameshifts and gene fusions. In pediatric cancers, fusions are a significant class of oncogenic drivers [44]. For instance, fusions of KIAA1549-BRAF are particularly characteristic of pediatric low-grade gliomas [45]. Overall, the younger patients have the lowest mutational burden.

About 8% of childhood cancers are associated with a hereditary predisposition [43, 46]. In the context of the present invention, the presence of such an inherited gene must be ruled out in selecting a suitable T cell donor. This is accomplished by DNA sequencing of PBMCs at the same time that the preliminary allele typing is done and before any cell culture with neoepitope peptides is conducted. Hereditary mutations are particularly found among the DNA repair genes (such as MSH, BRCA etc.), however as these result in a higher mutational level, the tumors they lead to are also more responsive to checkpoint inhibition than low mutational tumors and the secondary mutations these defects lead to are potential targets for neoepitope vaccines. Li-Fraumeni syndrome is an example of another type of germline predisposition due to a hereditary TP53 mutation [47]. The majority of pediatric cancers are the result of early somatic mutations rather than hereditary mutant genes. Of the somatic mutations, TP53 is also the most commonly found, along with KRAS, ATRX, NF1, and RBI [43]. Among single driver mutations commonly reported across all tumors pediatric tumors typically only exhibit one such driver mutation and often not accompanied by the many passenger mutations that are seen in adult tumors [43]. This paucity of mutations, and hence immunologic targets, underscores the difficulty and the necessity of precisely identifying appropriate neoepitope targets in pediatric tumors. It also indicates that immune escape arising from thymic tolerance may be a major contributing factor. Nevertheless, neoepitopes have been identified in pediatric cancers [44, 49] and posited as potential vaccinal targets.

Pediatric cancers are typically “immunologically cold” with little or no inflammation or T cell infiltration [28]. This is the combined result of tolerance, low mutation burden and hence low neoepitope numbers, and modulation of MHC expression. This limits the utility of immunotherapeutic interventions like checkpoint inhibitors, while young age also limits use of radiation and cytotoxic chemotherapy. However, precise neoepitope strategies have shown some feasibility in at least one instance [49].

What is needed in the art are improved methods of treating pediatric cancers.

### SUMMARY OF THE INVENTION

The methods of the present invention begin with two steps. In the first instance the sequences of normal and tumor tissue, obtained as a biopsy from the subject affected by cancer, are compared to identify mutated amino acids in the tumor and the T cell exposed amino acid motifs which contain the mutated amino acids. Secondly, the MHC alleles of the subject are determined and compared with those of a potential donor to identify a donor with matching alleles. In subsequent steps the binding affinity of peptides comprising the mutated amino acids is determined and an array of alternate peptides is generated in which the T cell exposed motifs are maintained constant, but the other amino acids are substituted, in order to generate an array of alternative peptides with a desired binding affinity to the MHC alleles of the cancer affected subject that are shared with the donor. A group of one or more peptides is selected from the array of alternative peptides, each having a desired binding affinity to the MHC allele that the donor has in common with the patient while maintaining the tumor specific T cell exposed motif. Antigen presenting cells, including but not limited to dendritic cells, are contacted with the selected alternative peptides, or with nucleic acids encoding the said peptides, such that the peptide is presented by the MHC of the antigen presenting cells. The dendritic cells carrying the selected peptide bound in its MHC molecules are then contacted by T cells harvested from the donor. Stimulated T cells responding to the peptide of interest presented by the dendritic cell MHC are expanded and infused into the cancer affected subject.

In some embodiments the group of selected peptides is selected to bind at a desired affinity with MHC I allele molecules: in other embodiments the group of selected peptides is selected to bind at a desired affinity with MHC II allele molecules. Therefore, in some embodiments, the T cell exposed motif that is maintained constant comprises 5 sequential amino acids to engage a CD8+ T cell, whereas in other embodiments the T cell exposed motif that is maintained constant comprises a discontinuous set of 5 amino acids to engage a CD4+ T cell. In some embodiments the selected peptides having a desired binding affinity are selected to bind with an affinity of less than 500 nanomolar, whereas in other embodiments the selection is for peptides that bind at an affinity of less than 200 nanomolar. In yet other preferred embodiments the preferred affinity is less than 100 nanomolar or less than 50 nanomolar.

In a preferred embodiment the donor is an MHC allelic haplomatch for the cancer affected subject, sharing 50% or more of the alleles. In yet other embodiments the donor matches multiple MHC alleles of the recipient subject: in preferred embodiments said sharing exceeds 25% of the alleles. In most preferred embodiments the donor is a family member, drawn from the group comprising a parent, child or sibling of the subject. In yet further preferred embodiments the donor shares microchimerism with the recipient subject. In some cases, said microchimerism arises when the recipient cancer subject carries non-inherited alleles shared with the donor, but the converse is also a desired embodiment in which the donor carries the non-inherited alleles of the recipient. Antigen presenting cells, and in particular cases dendritic cells are cultured and matured in vitro and provided with the selected peptides or nucleic acids encoding the said peptides, to bind the peptide in MHC and present to the donor T cells as pMHC in vitro. In some preferred embodiments the dendritic cells are harvested from the donor. In yet other instances dendritic cells are collected from the cancer affected subject.

In some embodiments, peptide-responsive T cells infused into the subject are CD8+ cells: in other embodiments they are CD4+ cells. In preferred embodiments both CD8+ and CD4+ T cells responsive to selected peptides are infused into the subject. In yet further embodiments donor dendritic cells may also be included in the infusion. In preferred embodiment the T cells that are responsive to the selected peptides of interest are separated from non-responsive peptides prior to infusion. In some particular instances the peptide-stimulated T cells may be separated from non-stimulated T cells by contacting them with antibody to CD137, or beads bearing the said antibody to separate activated T cells.

In some embodiments the potential response of donor cells is enhanced by stimulating the T cell repertoire of the donor in vivo prior to collection of T cells. In instances where potential cross-reactive autoimmunity is considered a de minimus risk, the donor may be vaccinated with the neoepitopes, as the selected peptides, in advance of collection of T cells. In other embodiments a more general approach is taken in which the donor's T cell repertoire is stimulated and diversified by administration of IVIG in advance of cell donation. In other embodiments the donor repertoire may be stimulated and diversified by other immunomodulatory interventions, including but not limited to, dietary supplement including immunoglobulins, or gastrointestinal microbiome inoculations.

In some embodiments following the infusion of epitope specific T cells into the cancer affected subject, said subject may be directly vaccinated with the same neoepitopes, either as a formulation comprising peptides or nucleic acids encoding the peptides.

In each case, prior to applying the selected peptides comprising tumor-specific T cell exposed motifs to a subject or donor, a review is conducted to determine if there are potential matching T cell exposed motifs in other proteins, which in the context of the MHC binding profile of the subject have the potential to elicit an adverse autoimmune response. Said review is accomplished by searching a database of the human proteome to locate matched pentameric motifs and by evaluation of their potential presentation in the context of their flanking regions and the host alleles. The selected tumor specific peptides, or their encoding amino acids, are selected to avoid matches with critical protein functions, as well as by evaluating their predicted binding to the subject's HLA alleles. In the case where a donor is vaccinated in advance of T cell collection, the corresponding T cell matches in conjunction with binding to any of the donor alleles is also evaluated to determine the risk profile: the donor may of course carry alleles which are not found in the cancer subject and so this is a necessary further separate review. Furthermore, in preferred embodiments the exome of the donor is screened for the tumor mutations found in the subject to identify and rule out hereditary mutations.

Once the subject has received the infusion of tumor specific donor T cells, said subject, in preferred embodiments, may be further treated with an immunomodulatory intervention selected to further boost the tumor specific response and extend the useful life of the mutation specific T cells. This may comprise a checkpoint inhibitor or other immune agonist.

In some applications of the present invention the subject affected by cancer is an adult over 25 years of age. In yet other embodiments the subject may be under 25 years of age or under 15 years of age and may be affected by a pediatric cancer. Said pediatric cancer may be from the group comprising medulloblastoma, neuroblastoma rhabdomyosarcoma. Ewing's sarcoma, osteosarcoma and Wilms tumor, or may be yet another pediatric cancer. Provided herein are specific peptides which are examples of the selected peptides that may be applicable to certain cancers and which are enumerated in the Examples, but are not considered limiting.

The peptides, selected and designed to bind with a desired affinity, may be delivered to the antigen presenting cells, including but not limited to the dendritic cells in vitro, in a number of modalities. The selected peptide may be contacted directly with the dendritic cell, but in some embodiments the selected peptide or peptides may be formulated as a particulate which may be a liposome, a nanoparticle, virosome or virus like particle, or the peptide may be embodied into a lipid drug delivery system. In yet other preferred embodiments the peptide of interest may be operatively linked to an immunoglobulin Fc receptor to facilitate uptake by dendritic cells. In further preferred embodiments the peptide, or a nucleic acid that encodes said peptide, may be inserted into the dendritic cell by electroporation. The selected peptides may be encoded in nucleic acid and, in some instances, delivered to the dendritic cell as a messenger RNA, plasmid, virus like particle or as a viral vector, which in some embodiments may be pseudotyped to facilitate dendritic cell uptake.

In some preferred embodiments, the present invention provides methods of treating a subject affected by cancer by providing tumor-mutation specific T cells from an MHC allele-matched donor, comprising the following steps: obtaining a biopsy of the subject's tumor:

obtaining sequences for proteins in said biopsy; identifying proteins from the biopsy containing mutated amino acids and the peptide comprising each of said mutated amino acids; determining T cell exposed motifs which comprise mutated amino acids in each of the proteins; determining the predicted binding affinity to the subject's MHC alleles of peptides which comprises each of said T cell exposed motifs comprising mutated amino acids, or a subset thereof; generating an array of alternative peptides not present in the tumor, wherein each peptide in the array comprises the amino acids of one of said T cell exposed motifs, and in which the amino acids not within the T cell exposed motif are substituted to change the predicted MHC binding affinity; selecting a group of one or more selected peptides from said array of alternative peptides which have a desired predicted binding affinity for one or more of the subject's MHC alleles; and synthesizing said group of one or more selected peptides, or nucleic acids encoding the selected peptides; and obtaining T cells from a donor who carries at least one matched MHC allele to the subject; and contacting dendritic cells in vitro with said selected peptides, or nucleic acids encoding the selected peptide, then contacting said dendritic cells with the T cells from said donor; and multiplying in vitro the T cells responsive to the selected peptide; and infusing said T cells responsive to the selected peptide from said donor into the subject.

In some preferred embodiments, the subject's MHC alleles are MHC I alleles. In some preferred embodiments, the subject's MHC alleles are MHC II alleles. In some preferred embodiments, the T cell exposed motifs comprise 5 sequential amino acids that engage the T cell receptor of a CD8+ T cell. In some preferred embodiments, the T cell exposed motifs comprise a discontinuous sequence of 5 amino acids that engage the T cell receptor of a CD4+ T cell.

In some preferred embodiments, the desired predicted binding affinity for said one or more MHC alleles is less than 500 nanomolar. In some preferred embodiments, the desired predicted binding affinity for said one or more MHC alleles is less than 200 nanomolar. In some preferred embodiments, the desired predicted binding affinity for said one or more MHC alleles is less than 100 nanomolar. In some preferred embodiments, the desired predicted binding affinity for said one or more MHC alleles is less than 50 nanomolar.

In some preferred embodiments, the donor carries one or more MHC I alleles matched to those of the subject. In some preferred embodiments, the donor carries one or more MHC II alleles matched to those of the subject. In some preferred embodiments, the donor is selected from the group consisting of a parent, a sibling, and a child of the subject. In some preferred embodiments, the donor and subject are matched in at least 25% of their MHC alleles. In some preferred embodiments, the donor and subject are matched in at least 50% of their MHC alleles. In some preferred embodiments, the donor carries microchimeric non-inherited MHC alleles shared with the subject. In some preferred embodiments, the subject carries microchimeric non-inherited MHC alleles shared with the donor.

In some preferred embodiments, the dendritic cells are drawn from the subject. In some preferred embodiments, the dendritic cells are drawn from the donor.

In some preferred embodiments, the T cells responsive to the selected peptide infused into the subject are CD8+ T cells. In some preferred embodiments, the T cells responsive to the selected peptide infused into the subject are CD4+ T cells. In some preferred embodiments, the T cells responsive to the selected peptide infused into the subject comprise both CD8+ and CD4+ T cells. In some preferred embodiments, the T cells responsive to the selected peptide infused into the subject comprise both CD8+ and CD4+ T cells and also donor dendritic cells. In some preferred embodiments, the T cells responsive to the selected peptide are preferentially separated from non-responsive T cells prior to infusion to the subject. In some preferred embodiments, the separation is achieved by panning with antibody to CD137.

In some preferred embodiments, the T cell repertoire of the donor is modified prior to collection of T cells. In some preferred embodiments, the donor is vaccinated with the selected peptides as described above prior to donation of cells. In some preferred embodiments, the methods further comprise analyzing the selected peptides to reduce the potential of adverse off-target binding in the donor, the analysis comprising: searching a reference database of T cell exposed motifs in the human proteome to identify T cell exposed motifs that match the T cell exposed motifs which comprise mutated amino acids in the selected peptides; determining the predicted MHC binding of the peptides in the human proteome which comprise the T cell exposed motifs; determining if there is high predicted binding to the donor's MHC alleles; evaluating if the peptides in the human proteome occur in proteins which constitute a risk of adverse reactions if targeted by a T cell; and eliminating any selected peptide comprising T cell exposed motifs giving rise to the risk. In some preferred embodiments, the donor receives IVIG prior to donation of cells. In some preferred embodiments, the donor receives an immunomodulatory intervention prior to donation of cells. In some preferred embodiments, the immunomodulatory intervention is a dietary supplement comprising immunoglobulin. In some preferred embodiments, the donor receives a gastrointestinal microbiome inoculation prior to donation of cells.

In some preferred embodiments, the infusion of the subject with the T cells responsive to the selected peptides is followed by vaccination of the subject with one or more of the selected peptides or nucleic acids encoding the peptides. In some preferred embodiments, a tissue sample from the donor is sequenced to determine if the tumor mutations found in the subject are hereditary.

In some preferred embodiments, the methods further comprise analyzing the selected peptides to reduce the potential of adverse off-target binding in the subject, the analysis comprising: searching a reference database of T cell exposed motifs in the human proteome to identify T cell exposed motifs that match the T cell exposed motifs which comprise mutated amino acids in the subject; determining the predicted MHC binding of the peptides in the human proteome which comprise the T cell exposed motifs; determining if there is high predicted binding to the subject's MHC alleles; evaluating if the peptides in the human proteome occur in proteins which constitute a risk of adverse reactions if targeted by a T cell; and eliminating any selected peptide comprising T cell exposed motifs giving rise to the risk.

In some preferred embodiments, the infusion of the subject with the T cells responsive to the selected peptides is followed by treatment of the subject with an immunomodulatory intervention. In some preferred embodiments, the immunomodulatory intervention is a checkpoint inhibitor.

In some preferred embodiments, the subject is over 25 years of age. In some preferred embodiments, the subject is under 25 years of age. In some preferred embodiments, the subject is under 15 years of age.

In some preferred embodiments, the tumor is a solid tumor. In some preferred embodiments, the tumor is a pediatric tumor. In some preferred embodiments, The pediatric tumor is selected from the group consisting of medulloblastoma, neuroblastoma, rhabdomyosarcoma. Ewing's sarcoma, osteosarcoma and Wilms' tumor.

In some preferred embodiments, the subject is affected by a glioblastoma and selected peptides are selected from the group consisting of SEQ ID NOs: 33-53 and SEQ ID NOs: 126-161. In some preferred embodiments, the subject is affected by a glioblastoma and the selected peptides comprises a T cell exposed motif selected from the group consisting of SEQ ID NOs: 1-32 and SEQ ID NOs: 54-125. In some preferred embodiments, the subject is affected by a low-grade glioma and selected peptides are selected from the group consisting of SEQ ID NOs: 185-255 and SEQ ID NOs: 275-279. In some preferred embodiments, the subject is affected by a by a low-grade glioma and the selected peptides comprises a T cell exposed motif selected from the group consisting of SEQ ID NOs: 163-173 and SEQ ID NOs: 263-264.

In some preferred embodiments, the selected peptide, or nucleic acid encoding the selected peptide, is in a particulate form when contacted with dendritic cells. In some preferred embodiments, the particulate form is selected from the group consisting of a liposome, nanoparticle, virosome, virus like particle, viral vector, pseudotyped viral vector and a lipid drug delivery system.

In some preferred embodiments, the selected peptide is operationally linked to a Fc receptor when contacted with dendritic cells. In some preferred embodiments, the selected peptide, or a nucleic acid encoding the selected peptide, is introduced to the dendritic cell by electroporation.

The description and examples below provide more detail as to the methods embodied by this invention.

### DESCRIPTION OF FIGURES

FIG. 1: Distribution histograms of TCEM I frequency for the 37,622 different TCEM peptides mutants (top panel) and wildtype motifs (bottom panel) in seven proteins of interest as listed in FIGS. 1 and 2. The base frequency of the TCEM in the proteome was log 2 basis. This frequency was standardized to a zero mean unit variance distribution with a Johnson S1 distribution function. The wildtype distribution shows that the mean is shifted slightly negative from zero mean of the full proteome but the standard deviation is very nearly 1.0 (unit variance). The wildtype TCEM frequency is a relatively random selection from the proteome unit variance distribution. The histogram bar at the far left of the top panel is a coded frequency for TCEM completely absent from the human proteome. This pattern of TCEM generation by mutation shows the stochastic mutation process inserts amino acids into protein sequences that are either much more rare or in many cases (14% overall), completely absent in normal protein sequences in the proteome.

FIG. 2: T cell repertoire diversity by decade of age. Analysis of 666 patients. Log₂ cellular frequency distributions were modeled as a four normal distribution mixture. The boundary line represents the fit of the model. Probability represents the fraction of the total cells in the repertoire in that particular Log₂ frequency bin.

FIG. 3: Individual T cell repertoire diversity within the decade 61-70 years of age shown in aggregate in FIG. 2.

FIG. 4: Example of HLA typing data derived by the exact aligner method shown in Example 4.

FIG. 5: Examples of rare and less common motifs generated by mutation sin common oncogenes and tumor suppressor proteins. Y axis shows the frequency of each motif relative to a mean of zero in the whole human proteome. Lower values are thus more rare. Top tier shows TCEM I, middle tier TCEM IIA and lower tier TCEM IIB. On the X axis peptides are aligned at the mutation position indicated by the vertical line at zero and motif frequency is shown for peptides in position upstream and downstream of that position.

FIG. 6: In a series of 33 glioblastoma patients the characteristics of peptides bearing amino acid mutations were evaluated. FIG. 6A shows that the predicted MHC I binding affinity of a 9 peptide comprising the mutant amino acid is unchanged relative to its wildtype homolog, regardless of whether the mutant amino acid is exposed or in the groove exposed or pocket position. FIG. 6B show that across over 5000 mutations in the 33 cases mutations were more likely to favor binding in pocket positions than non-pocket exposed positions.

### DEFINITIONS

As used herein, the term “genome” refers to the genetic material (e.g., chromosomes) of an organism or a host cell.

As used herein, the term “proteome” refers to the entire set of proteins expressed by a genome, cell, tissue or organism. A “partial proteome” refers to a subset the entire set of proteins expressed by a genome, cell, tissue or organism. Examples of “partial proteomes” include, but are not limited to, transmembrane proteins, secreted proteins, and proteins with a membrane motif. Human proteome refers to all the proteins comprised in a human being. Multiple such sets of proteins have been sequenced and are accessible at the InterPro international repository (www.ebi.ac.uk/interpro). Human proteome is also understood to include those proteins and antigens thereof which may be over-expressed in certain pathologies, or expressed in a different isoforms in certain pathologies. Hence, as used herein, tumor associated antigens are considered part of the human proteome. “Proteome” may also be used to describe a large compilation or collection of proteins, such as all the proteins in an immunoglobulin collection or a T cell receptor repertoire, or the proteins which comprise a collection such as the allergome, such that the collection is a proteome which may be subject to analysis. All the proteins in a bacteria or other microorganism are considered its proteome.

As used herein, the terms “protein,” “polypeptide,” and “peptide” refer to a molecule comprising amino acids joined via peptide bonds. In general “peptide” is used to refer to a sequence of 40 or less amino acids and “polypeptide” is used to refer to a sequence of greater than 40 amino acids.

As used herein, the term, “synthetic polypeptide,” “synthetic peptide” and “synthetic protein” refer to peptides, polypeptides, and proteins that are produced by a recombinant process (i.e., expression of exogenous nucleic acid encoding the peptide, polypeptide or protein in an organism, host cell, or cell-free system) or by chemical synthesis.

As used herein, the term “protein of interest” refers to a protein encoded by a nucleic acid of interest. It may be applied to any protein to which further analysis is applied or the properties of which are tested or examined. Similarly, as used herein, “target protein” may be used to describe a protein of interest that is subject to further analysis.

As used herein the term “amino acid of interest” refers to an amino acid which sets the protein apart from other sequences of the same protein, for instance by being the product of a mutation, indel, splice or fusion event, or the amino acid attracts attention as it is a salient feature in a particular T cell epitope.

A “target peptide” as used herein is one to which it is desired to direct an immune response.

As used herein “peptidase” refers to an enzyme which cleaves a protein or peptide. The term peptidase may be used interchangeably with protease, proteinases, oligopeptidases, and proteolytic enzymes. Peptidases may be endopeptidases (endoproteases), or exopeptidases (exoproteases). The term peptidase would also include the proteasome which is a complex organelle containing different subunits each having a different type of characteristic scissile bond cleavage specificity. Similarly the term peptidase inhibitor may be used interchangeably with protease inhibitor or inhibitor of any of the other alternate terms for peptidase.

As used herein, the term “exopeptidase” refers to a peptidase that requires a free N-terminal amino group, C-terminal carboxyl group or both, and hydrolyses a bond not more than three residues from the terminus. The exopeptidases are further divided into aminopeptidases, carboxypeptidases, dipeptidyl-peptidases, peptidyl-dipeptidases, tripeptidyl-peptidases and dipeptidases.

As used herein, the term “endopeptidase” refers to a peptidase that hydrolyses internal, alpha-peptide bonds in a polypeptide chain, tending to act away from the N-terminus or C-terminus. Examples of endopeptidases are chymotrypsin, pepsin, papain and cathepsins. A very few endopeptidases act a fixed distance from one terminus of the substrate, an example being mitochondrial intermediate peptidase. Some endopeptidases act only on substrates smaller than proteins, and these are termed oligopeptidases. An example of an oligopeptidase is thimet oligopeptidase. Endopeptidases initiate the digestion of food proteins, generating new N- and C-termini that are substrates for the exopeptidases that complete the process. Endopeptidases also process proteins by limited proteolysis. Examples are the removal of signal peptides from secreted proteins (e.g. signal peptidase I,) and the maturation of precursor proteins (e.g. enteropeptidase, furin). In the nomenclature of the Nomenclature Committee of the International Union of Biochemistry and Molecular Biology (NC-IUBMB) endopeptidases are allocated to sub-subclasses EC 3.4.21, EC 3.4.22, EC 3.4.23, EC 3.4.24 and EC 3.4.25 for serine-, cysteine-, aspartic-, metallo- and threonine-type endopeptidases, respectively. Endopeptidases of particular interest are the cathepsins, and especially cathepsin B, L and S known to be active in antigen presenting cells.

As used herein, the term “immunogen” refers to a molecule which stimulates a response from the adaptive immune system, which may include responses drawn from the group comprising an antibody response, a cytotoxic T cell response, a T helper response, and a T cell memory. An immunogen may stimulate an upregulation of the immune response with a resultant inflammatory response, or may result in down regulation or immunosuppression. Thus the T-cell response may be a T regulatory response. An immunogen also may stimulate a B-cell response and lead to an increase in antibody titer. Another term used herein to describe a molecule or combination of molecules which stimulate an immune response is “antigen”.

As used herein, the term “native” (or wild type) when used in reference to a protein refers to proteins encoded by the genome of a cell, tissue, or organism, other than one manipulated to produce synthetic proteins.

As used herein the term “epitope” refers to a peptide sequence which elicits an immune response, from either T cells or B cells or antibody

As used herein, the term “B-cell epitope” refers to a polypeptide sequence that is recognized and bound by a B-cell receptor. A B-cell epitope may be a linear peptide or may comprise several discontinuous sequences which together are folded to form a structural epitope. Such component sequences which together make up a B-cell epitope are referred to herein as B-cell epitope sequences. Hence, a B-cell epitope may comprise one or more B-cell epitope sequences. Hence, a B cell epitope may comprise one or more B-cell epitope sequences. A linear B-cell epitope may comprise as few as 2-4 amino acids or more amino acids.

“B cell core peptides” or “core pentamer” when used herein refers to the central 5 amino acid peptide in a predicted B cell epitope sequence. The B cell epitope may be evaluated by predicting the binding of across a series of 9-mer windows, the core pentamer then is the central pentamer of the 9-mer window.

As used herein, the term “predicted B-cell epitope” refers to a polypeptide sequence that is predicted to bind to a B-cell receptor by a computer program, for example, as described in PCT US2011/029192, PCT US2012/055038, US2014/014523, PCT US2015/039969, PCT US2020/037206, U.S. Pat. No. 10,706,955 and 10,755,801 each of which is incorporated herein by reference in its entirety, and in addition by Bepipred (Larsen, et al., Immunome Research 2:2, 2006.) and others as referenced by Larsen et al (ibid) (Hopp T et al PNAS 78:3824-3828, 1981; Parker J et al, Biochem. 25:5425-5432, 1986). A predicted B-cell epitope may refer to the identification of B-cell epitope sequences forming part of a structural B-cell epitope or to a complete B-cell epitope.

As used herein, the term “T-cell epitope” refers to a polypeptide sequence which when bound to a major histocompatibility protein molecule provides a configuration recognized by a T-cell receptor. Typically, T-cell epitopes are presented bound to an MHC molecule on the surface of an antigen-presenting cell.

As used herein, the term “predicted T-cell epitope” refers to a polypeptide sequence that is predicted to bind to a major histocompatibility protein molecule by the neural network algorithms described herein, by other computerized methods, or as determined experimentally.

As used herein, the term “major histocompatibility complex (MHC)” refers to the MHC Class I and MHC Class II genes and the proteins encoded thereby. Molecules of the MHC bind small peptides and present them on the surface of cells for recognition by T-cell receptor-bearing T-cells. The MHC is both polygenic (there are several MHC class I and MHC class II genes) and polyallelic or polymorphic (there are multiple alleles of each gene). The terms MHC-I, MHC-II, MHC-1 and MHC-2 are variously used herein to indicate these classes of molecules. Included are both classical and nonclassical MHC molecules. An MHC molecule is made up of multiple chains (alpha and beta chains) which associate to form a molecule. The MHC molecule contains a cleft or groove which forms a binding site for peptides. Peptides bound in the cleft or groove may then be presented to T-cell receptors. The term “MHC binding region” refers to the groove region of the MHC molecule where peptide binding occurs.

As used herein, a “MHC II binding groove” refers to the structure of an MHC molecule that binds to a peptide. The peptide that binds to the MHC II binding groove may be from about 11 amino acids to about 23 amino acids in length, but typically comprises a 15-mer. The amino acid positions in the peptide that binds to the groove are numbered based on a central core of 9 amino acids numbered 1-9, and positions outside the 9 amino acid core numbered as negative (N terminal) or positive (C terminal). Hence, in a 15mer the amino acid binding positions are numbered from −3 to +3 or as follows: −3, −2, −1, 1, 2, 3, 4, 5, 6, 7, 8, 9, +1, +2, +3.

As used herein, the term “haplotype” refers to the HLA alleles found on one chromosome and the proteins encoded thereby. Haplotype may also refer to the allele present at any one locus within the MHC. Each class of MHC-Is represented by several loci: e.g., HLA-A (Human Leukocyte Antigen-A), HLA-B, HLA-C, HLA-E, HLA-F, HLA-G, HLA-H, HLA-J. HLA-K, HLA-L, HLA-P and HLA-V for class I and HLA-DRA, HLA-DRB1-9, HLA-, HLA-DQA1, HLA-DQB1, HLA-DPA1, HLA-DPB1, HLA-DMA, HLA-DMB, HLA-DOA, and HLA-DOB for class II. The terms “HLA allele” and “MHC allele” are used interchangeably herein. HLA alleles are listed at hla.alleles.org/nomenclature/naming.html, which is incorporated herein by reference.

The MHCs exhibit extreme polymorphism: within the human population there are, at each genetic locus, a great number of haplotypes comprising distinct alleles—the IMGT/HLA database release (February 2010) lists 948 class I and 633 class II molecules, many of which are represented at high frequency (>1%). MHC alleles may differ by as many as 30-aa substitutions. Different polymorphic MHC alleles, of both class I and class II, have different peptide specificities; each allele encodes proteins that bind peptides exhibiting particular sequence patterns.

The naming of new HLA genes and allele sequences and their quality control is the responsibility of the WHO Nomenclature Committee for Factors of the HLA System, which first met in 1968, and laid down the criteria for successive meetings. This committee meets regularly to discuss issues of nomenclature and has published 19 major reports documenting firstly the HLA antigens and more recently the genes and alleles. The standardization of HLA antigenic specifications has been controlled by the exchange of typing reagents and cells in the International Histocompatibility Workshops. The IMGT/HLA Database collects both new and confirmatory sequences, which are then expertly analyzed and curated before been named by the Nomenclature Committee. The resulting sequences are then included in the tools and files made available from both the IMGT/HLA Database and at hla.alleles.org.

Each HLA allele name has a unique number corresponding to up to four sets of digits separated by colons. See e.g., hla.alleles.org/nomenclature/naming.html which provides a description of standard HLA nomenclature and Marsh et al., Nomenclature for Factors of the HLA System, 2010 Tissue Antigens 2010 75:291-455. HLA-DRB1*13:01 and HLA-DRB1*13:01:01:02 are examples of standard HLA nomenclature. The length of the allele designation is dependent on the sequence of the allele and that of its nearest relative. All alleles receive at least a four digit name, which corresponds to the first two sets of digits, longer names are only assigned when necessary.

The digits before the first colon describe the type, which often corresponds to the serological antigen carried by an allele, The next set of digits are used to list the subtypes, numbers being assigned in the order in which DNA sequences have been determined. Alleles whose numbers differ in the two sets of digits must differ in one or more nucleotide substitutions that change the amino acid sequence of the encoded protein. Alleles that differ only by synonymous nucleotide substitutions (also called silent or non-coding substitutions) within the coding sequence are distinguished by the use of the third set of digits. Alleles that only differ by sequence polymorphisms in the introns or in the 5′ or 3′ untranslated regions that flank the exons and introns are distinguished by the use of the fourth set of digits. In addition to the unique allele number there are additional optional suffixes that may be added to an allele to indicate its expression status. Alleles that have been shown not to be expressed, ‘Null’ alleles have been given the suffix ‘N’. Those alleles which have been shown to be alternatively expressed may have the suffix ‘L’, ‘S’, ‘C’, ‘A’ or ‘Q’. The suffix ‘L’ is used to indicate an allele which has been shown to have ‘Low’ cell surface expression when compared to normal levels. The ‘S’ suffix is used to denote an allele specifying a protein which is expressed as a soluble ‘Secreted’ molecule but is not present on the cell surface. A ‘C’ suffix to indicate an allele product which is present in the ‘Cytoplasm’ but not on the cell surface. An ‘A’ suffix to indicate ‘Aberrant’ expression where there is some doubt as to whether a protein is expressed. A ‘Q’ suffix when the expression of an allele is ‘Questionable’ given that the mutation seen in the allele has previously been shown to affect normal expression levels.

In some instances, the HLA designations used herein may differ from the standard HLA nomenclature just described due to limitations in entering characters in the databases described herein. As an example, DRB1_0104, DRB1*0104, and DRB1-0104 are equivalent to the standard nomenclature of DRB1*01:04. In most instances, the asterisk is replaced with an underscore or dash and the semicolon between the two digit sets is omitted.

As used herein, the term “polypeptide sequence that binds to at least one major histocompatibility complex (MHC) binding region” refers to a polypeptide sequence that is recognized and bound by one or more particular MHC binding regions as predicted by the neural network algorithms described herein or as determined experimentally.

As used herein the terms “canonical” and “non-canonical” are used to refer to the orientation of an amino acid sequence. Canonical refers to an amino acid sequence presented or read in the N terminal to C terminal order: non-canonical is used to describe an amino acid sequence presented in the inverted or C terminal to N terminal order. “Canonical” is also used to designate the dominant sequence of a protein for which many isoforms exist. The canonical protein is thus typically that in the Reference sequence designated by uniport.org.

As used herein, the term “allergen” refers to an antigenic substance capable of producing immediate hypersensitivity and includes both synthetic as well as natural immunostimulant peptides and proteins. Allergen includes but is not limited to any protein or peptide catalogued in the Structural Database of Allergenic Proteins database http://fermi.utmb.edu/SDAP/index.html

As used herein, the term “transmembrane protein” refers to proteins that span a biological membrane. There are two basic types of transmembrane proteins. Alpha-helical proteins are present in the inner membranes of bacterial cells or the plasma membrane of eukaryotes, and sometimes in the outer membranes. Beta-barrel proteins are found only in outer membranes of Gram-negative bacteria, cell wall of Gram-positive bacteria, and outer membranes of mitochondria and chloroplasts.

As used herein, the term “affinity” refers to a measure of the strength of binding between two members of a binding pair, for example, an antibody and an epitope and an epitope and a MHC-I or II haplotype. K_{d} is the dissociation constant and has units of molarity. The affinity constant is the inverse of the dissociation constant. An affinity constant is sometimes used as a generic term to describe this chemical entity. It is a direct measure of the energy of binding. The natural logarithm of K is linearly related to the Gibbs free energy of binding through the equation ΔG₀=−RT LN(K) where R=gas constant and temperature is in degrees Kelvin. Affinity may be determined experimentally, for example by surface plasmon resonance (SPR) using commercially available Biacore SPR units (GE Healthcare) or in silico by methods such as those described herein in detail. Affinity may also be expressed as the ic50) or inhibitory concentration 50, that concentration at which 50% of the peptide is displaced. Likewise In(ic50) refers to the natural log of the ic50.

The term “K_{off}”, as used herein, is intended to refer to the off rate constant, for example, for dissociation of an antibody from the antibody/antigen complex, or for dissociation of an epitope from an MHC haplotype.

The term “K_{d}”, as used herein, is intended to refer to the dissociation constant (the reciprocal of the affinity constant “Ka”), for example, for a particular antibody-antigen interaction or interaction between an epitope and an MHC haplotype.

As used herein, the terms “strong binder” and “strong binding” and “High binder” and “high binding” or “high affinity” refer to a binding pair or describe a binding pair that have an affinity of greater than 2×10⁷M⁻¹ (equivalent to a dissociation constant of 50 nM Kd)

As used herein, the term “moderate binder” and “moderate binding” and “moderate affinity” refer to a binding pair or describe a binding pair that have an affinity of from 2×10⁷M⁻¹ to 2×10⁶ M⁻¹

As used herein, the terms “weak binder” and “weak binding” and “low affinity” refer to a binding pair or describe a binding pair that have an affinity of less than 2×10⁶M⁻¹ (equivalent to a dissociation constant of less than 500 nM Kd).

Binding affinity may also be expressed by the standard deviation from the mean binding found in the peptides making up a protein. Hence a binding affinity may be expressed as “−1σ” or <−1σ, where this refers to a binding affinity of 1 or more standard deviations below the mean. A common mathematical transformation used in statistical analysis is a process called standardization wherein the distribution is transformed from its standard units to standard deviation units where the distribution has a mean of zero and a variance (and standard deviation) of 1. Because each protein comprises unique distributions for the different MHC alleles standardization of the affinity data to zero mean and unit variance provides a numerical scale where different alleles and different proteins can be compared. Analysis of a wide range of experimental results suggest that a criterion of standard deviation units can be used to discriminate between potential immunological responses and non-responses. An affinity of 1 standard deviation below the mean was found to be a useful threshold in this regard and thus approximately 15% (16.2% to be exact) of the peptides found in any protein will fall into this category.

The terms “specific binding” or “specifically binding” when used in reference to the interaction of an antibody and a protein or peptide or an epitope and an MHC haplotype means that the interaction is dependent upon the presence of a particular structure (i.e., the antigenic determinant or epitope) on the protein: in other words the antibody is recognizing and binding to a specific protein structure rather than to proteins in general. For example, if an antibody is specific for epitope “A.” the presence of a protein containing epitope A (or free, unlabeled A) in a reaction containing labeled “A” and the antibody will reduce the amount of labeled A bound to the antibody.

As used herein, the term “antigen binding protein” refers to proteins that bind to a specific antigen. “Antigen binding proteins” include, but are not limited to, immunoglobulins, including polyclonal, monoclonal, chimeric, single chain, and humanized antibodies, Fab fragments, F(ab′)2 fragments, and Fab expression libraries. Various procedures known in the art are used for the production of polyclonal antibodies. For the production of antibody, various host animals can be immunized by injection with the peptide corresponding to the desired epitope including but not limited to rabbits, mice, rats, sheep, goats, etc.

“Adjuvant” as used herein encompasses various adjuvants that are used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, squalene, squalene emulsions, liposomes, imiquimod, keyhole limpet hemocyanins, dinitrophenol, and potentially useful human adjuvants such as BCG (Bacille Calmette-Guerin) and Corynebacterium parvum. In other embodiments a cytokine may be co-administered, including but not limited to interferon gamma or stimulators thereof, interleukin 12, or granulocyte stimulating factor. In other embodiments the peptides or their encoding nucleic acids may be co-administered with a local inflammatory agent, either chemical or physical. Examples include, but are not limited to, heat, infrared light, proinflammatory drugs, including but not limited to imiquimod.

As used herein “immunoglobulin” means the distinct antibody molecule secreted by a clonal line of B cells: hence when the term “100 immunoglobulins” is used it conveys the distinct products of 100 different B-cell clones and their lineages.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refer to a device that is able to read a program from a computer memory (e.g., ROM or other computer memory) and perform a set of steps according to the program.

As used herein, the term “support vector machine” refers to a set of related supervised learning methods used for classification and regression. Given a set of training examples, each marked as belonging to one of two categories, an SVM training algorithm builds a model that predicts whether a new example falls into one category or the other.

As used herein, the term “classifier” when used in relation to statistical processes refers to processes such as neural nets and support vector machines.

As used herein “neural net”, which is used interchangeably with “neural network” and sometimes abbreviated as NN, refers to various configurations of classifiers used in machine learning, including multilayered perceptrons with one or more hidden layer, support vector machines and dynamic Bayesian networks. These methods share in common the ability to be trained, the quality of their training evaluated, and their ability to make either categorical classifications of non-numeric data or to generate equations for predictions of continuous numbers in a regression mode. Perceptron as used herein is a classifier which maps its input x to an output value which is a function of x, or a graphical representation thereof.

As used herein, the term “principal component analysis”, or as abbreviated “PCA”, refers to a mathematical process which reduces the dimensionality of a set of data (Wold, S., Sjorstrom,M., and Eriksson, L., Chemometrics and Intelligent Laboratory Systems 2001.58: 109-130.; Multivariate and Megavariate Data Analysis Basic Principles and Applications (Parts I&II) by L. Eriksson, E. Johansson, N. Kettaneh-Wold, and J. Trygg, 2006 2^{nd} Edit. Umetrics Academy). Derivation of principal components is a linear transformation that locates directions of maximum variance in the original input data, and rotates the data along these axes. For n original variables, n principal components are formed as follows: The first principal component is the linear combination of the standardized original variables that has the greatest possible variance. Each subsequent principal component is the linear combination of the standardized original variables that has the greatest possible variance and is uncorrelated with all previously defined components. Further, the principal components are scale-independent in that they can be developed from different types of measurements. The application of PCA generates numerical coefficients (descriptors). The coefficients are effectively proxy variables whose numerical values are seen to be related to underlying physical properties of the molecules. A description of the application of PCA to generate descriptors of amino acids and by combination thereof peptides is provided in PCT US2011/029192 incorporated herein by reference in its entirety. Unlike neural nets PCA do not have any predictive capability. PCA is deductive not inductive.

As used herein, the term “vector” when used in relation to a computer algorithm or the present invention, refers to the mathematical properties of the amino acid sequence.

As used herein, the term “vector,” when used in relation to recombinant DNA technology, refers to any genetic element, such as a plasmid, phage, transposon, cosmid, chromosome, retrovirus, virion, etc., which is capable of replication when associated with the proper control elements and which can transfer gene sequences between cells. Thus, the term includes cloning and expression vehicles, as well as viral vectors. “Viral vector” as used herein includes but is not limited to adenoviral vectors, adeno-associated viral vectors, lentiviral vectors, retroviral vectors, poliovirus vectors, measles virus vectors, flavivirus vectors, poxvirus vectors, and other viral vectors which may be used to deliver a peptide or nucleic acid sequence to a host cell.

As used herein, the term “host cell” refers to any eukaryotic cell (e.g., mammalian cells, avian cells, amphibian cells, plant cells, fish cells, insect cells, yeast cells), and bacteria cells, and the like, whether located in vitro or in vivo (e.g., in a transgenic organism).

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acids are nucleic acids present in a form or setting that is different from that in which they are found in nature. In contrast, non-isolated nucleic acids are nucleic acids such as DNA and RNA that are found in the state in which they exist in nature.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

A “subject” is an animal such as vertebrate, preferably a mammal such as a human, a bird, or a fish. Mammals are understood to include, but are not limited to, murines, simians, humans, bovines, ovines, cervids, equines, porcines, canines, felines etc.). A “subject affected by cancer” is a cancer patient.

An “effective amount” is an amount sufficient to effect beneficial or desired results. An effective amount can be administered in one or more administrations,

As used herein, the term “purified” or “to purify” refers to the removal of undesired components from a sample. As used herein, the term “substantially purified” refers to molecules, either nucleic or amino acid sequences, that are removed from their natural environment, isolated or separated, and are at least 60% free, preferably 75% free, and most preferably 90% free from other components with which they are naturally associated. An “isolated polynucleotide” is therefore a substantially purified polynucleotide.

As used herein “Complementarity Determining Regions” (CDRs) are those parts of the immunoglobulin variable chains which determine how these molecules bind to their specific antigen. Each immunoglobulin variable region typically comprises three CDRs and these are the most highly variable regions of the molecule. T cell receptors also comprise similar CDRs and the term CDR may be applied to T cell receptors.

As used herein, the term “motif” refers to a characteristic sequence of amino acids forming a distinctive pattern: this may also be expressed as an “amino acid motif”. A “pentamer motif” is a combination of five amino acids, either contiguous to each other or separated by one or more other amino acids

The term “Groove Exposed Motif” (GEM) as used herein refers to a subset of amino acids within a peptide that binds to an MHC molecule: the GEM comprises those amino acids which are turned inward towards the groove formed by the MHC molecule and which play a significant role in determining the binding affinity. In the case of human MHC-I the GEM amino acids are typically (1,2,3,9). In the case of MHC-II molecules two formats of GEM are most common comprising amino acids (−3,2,−1,1,4,6,9,+1,+2,+3) and (−3,2, 1,2,4,6,9,+1,+2,+3) based on a 15-mer peptide with a central core of 9 amino acids numbered 1-9) and positions outside the core numbered as negative (N terminal) or positive (C terminal). Groove exposed positions are also referred to herein as “pocket positions”. “Immunoglobulin germline” is used herein to refer to the variable region sequences encoded in the inherited germline genes and which have not yet undergone any somatic hypermutation. Each individual carries and expresses multiple copies of germline genes for the variable regions of heavy and light chains. These undergo somatic hypermutation during affinity maturation. Information on the germline sequences of immunoglobulins is collated and referenced by www.imgt.org [1]. “Germline family” as used herein refers to the 7 main gene groups, catalogued at IMGT, which share similarity in their sequences and which are further subdivided into subfamilies.

“Affinity maturation” is the molecular evolution that occurs during somatic hypermutation during which unique variable region sequences generated that are the best at targeting and neutralizing and antigen become clonally expanded and dominate the responding cell populations.

“Germline motif” as used herein describes the amino acid subsets that are found in germline immunoglobulins. Germline motifs comprise both GEM and TCEM motifs found in the variable regions of immunoglobulins which have not yet undergone somatic hypermutation.

“pMHC” Is used to describe a complex of a peptide bound to an MHC molecule. In many instances a peptide bound to an MHC-I will be a 9-mer or 10-mer however other sizes of 7-11 amino acids may be thus bound. Similarly MHC-II molecules may form pMHC complexes with peptides of 15 amino acids or with peptides of other sizes from 11-23 amino acids. The term pMHC is thus understood to include any short peptide bound to a corresponding MHC.

“Somatic hypermutation” (SHM), as used herein refers to the process by which variability in the immunoglobulin variable region is generated during the proliferation of individual B-cells responding to an immune stimulus. SHM occurs in the complementarity determining regions.

“T-cell exposed motif” (also where abbreviated TCEM), as used herein, refers to the sub set of amino acids in a peptide bound in a MHC molecule which are directed outwards and exposed to a T-cell binding to the pMHC complex. A T-cell binds to a complex molecular space-shape made up of the outer surface MHC of the particular HLA allele and the exposed amino acids of the peptide bound within the MHC. Hence any T-cell recognizes a space shape or receptor which is specific to the combination of HLA and peptide. The amino acids which comprise the TCEM in an MHC-I binding peptide typically comprise positions 4, 5, 6, 7, 8 of a 9-mer. The amino acids which comprise the TCEM in an MHC-II binding peptide typically comprise 2, 3, 5, 7, 8 or −1, 3, 5, 7, 8 based on a 15-mer peptide with a central core of 9 amino acids numbered 1-9 and positions outside the core numbered as negative (N terminal) or positive (C terminal). As indicated under pMHC, the peptide bound to an MHC may be of other lengths and thus the numbering system here is considered a non-exclusive example of the instances of 9-mer and 15 mer peptides.

As used herein “histotope” refers to the outward facing surface of the MHC molecules which surrounds the T cell exposed motif and in combination with the T cell exposed motif serves as the binding surface for the T cell receptor.

As used herein the T cell receptor refers to the molecules exposed on the surface of a T cell which engage the histotope of the MHC and the T cell exposed motif of a peptide bound in the MHC. The T cell receptor comprises two protein chains, known as the alpha and beta chain in 95% of human T cells and as the delta and gamma chains in the remaining 5% of human T cells. Each chain comprises a variable region and a constant region. Each variable region comprises three complementarity determining regions or CDRs

“Regulatory T-cell” or “Treg” as used herein, refers to a T-cell which has an immunosuppressive or down-regulatory function. Regulatory T-cells were formerly known as suppressor T-cells. Regulatory T-cells come in many forms but typically are characterized by expression CD4+, CD25, and Foxp3. Tregs are involved in shutting down immune responses after they have successfully eliminated invading organisms, and also in preventing immune responses to self-antigens or autoimmunity.

“uTOPE™ analysis” as used herein refers to the computer assisted processes for predicting binding of peptides to MHC and predicting cathepsin cleavage, described in PCT US2011/029192, PCT US2012/055038, US2014/014523, PCT US2015/039969, PCT US2020/037206, U.S. Pat. No. 10,706,955 and 10,755,801, each of which is incorporated herein by reference in its entirety.

“Framework region” as used herein refers to the amino acid sequences within an immunoglobulin variable region which do not undergo somatic hypermutation.

“Isotype” as used herein refers to the related proteins of particular gene family. Immunoglobulin isotype refers to the distinct forms of heavy and light chains in the immunoglobulins. In heavy chains there are five heavy chain isotypes (alpha, delta, gamma, epsilon, and mu, leading to the formation of IgA, IgD, IgG, IgE and IgM respectively) and light chains have two isotypes (kappa and lambda). Isotype when applied to immunoglobulins herein is used interchangeably with immunoglobulin “class”.

“Isoform” as used herein refers to different forms of a protein which differ in a small number of amino acids. The isoform may be a full-length protein (i.e., by reference to a reference wild-type protein or isoform) or a modified form of a partial protein, i.e., be shorter in length than a reference wild-type protein or isoform.

“Class switch recombination” (CSR) as used herein refers to the change from one isotype of immunoglobulin to another in an activated B cell, wherein the constant region associated with a specific variable region is changed, typically from IgM to IgG or other isotypes.

“Immunostimulation” as used herein refers to the signaling that leads to activation of an immune response, whether the immune response is characterized by a recruitment of cells or the release of cytokines which lead to suppression of the immune response. Thus, immunostimulation refers to both upregulation or down regulation.

“Up-regulation” as used herein refers to an immunostimulation which leads to cytokine release and cell recruitment tending to eliminate a non self or exogenous epitope. Such responses include recruitment of T cells, including effectors such as cytotoxic T cells, and inflammation. In an adverse reaction upregulation may be directed to a self-epitope.

“Down regulation” as used herein refers to an immunostimulation which leads to cytokine release that tends to dampen or eliminate a cell response. In some instances such elimination may include apoptosis of the responding T cells.

“Frequency class” or “frequency classification” as used herein is used to describe logarithmic based bins or subsets of amino acid motifs or cells. When applied to the counts of TCEM motifs found in a given dataset of peptides a logarithmic (log base 2) frequency categorization scheme was developed to describe the distribution of motifs in a dataset. As the cellular interactions between T-cells and antigen presenting cells displaying the motifs in MHC molecules on their surfaces are the ultimate result of the molecular interactions, using a log base 2 system implies that each adjacent frequency class would double or halve the cellular interactions with that motif. Thus, using such a frequency categorization scheme makes it possible to characterize subtle differences in motif usage as well as providing a comprehensible way of visualizing the cellular interaction dynamics with the different motifs. Hence a Frequency Class 2, or FC 2 means 1 in 4, a Frequency class 10 or FC 10 means 1 in 2¹⁰ or 1 in 1024. In other embodiments the frequency classification of the TCEM motif in the reference dataset is described by the quantile score of the TCEM in the reference dataset. Quantile scores are used, but is not limited to, applications where the reference dataset is the human proteome or a microbial proteome. “Frequency class” or “frequency classification” may also be applied to cellular clonotypic frequency where it refers to subgroups or bins defined by logarithmic based groupings, whether log base 2 or another selected log base.

A “rare TCEM” as used herein is one which is completely missing in the human proteome or present in up to only five instances in the human proteome.

“Adverse immune response” as used herein may refer to (a) the induction of immunosuppression when the appropriate response is an active immune response to eliminate a pathogen or tumor or (b) the induction of an upregulated active immune response to a self-antigen or (c) an excessive up-regulation unbalanced by any suppression, as may occur for instance in an allergic response.

“Clonotype” as used herein refers to the cell lineage arising from one unique cell. In the particular case of a B cell clonotype it refers to a clonal population of B cells that produces a unique sequence of IGV. The number of B cells that express that sequence varies from singletons to thousands in the repertoire of an individual. In the case of a T cell it refers to a cell lineage which expresses a particular TCR. A clonotype of cancer cells all arise from one cell and carry a particular mutation or mutations or the derivates thereof. The above are examples of clonotypes of cells and should not be considered limiting.

As used herein “epitope mimic” or “TCEM mimic” is used to describe a peptide which has an identical or overlapping TCEM, but may have a different GEM. Such a mimic occurring in one protein may induce an immune response directed towards another protein which carries the same TCEM motif. This may give rise to autoimmunity or inappropriate responses to the second protein.

“Cytokine” as used herein refers to a protein which is active in cell signaling and may include, among other examples, chemokines, interferons, interleukins, lymphokines, granulocyte colony-stimulating factor, tumor necrosis factor and programmed death proteins.

As used herein “oncoprotein” means a protein encoded by an oncogene which can cause the transformation of a cell into a tumor cell if introduced into it. Examples of oncoproteins include but are not limited to the early proteins of papillomaviruses, polyomaviruses, adenoviruses and herpesviruses, however oncoproteins are not necessarily of viral origin.

“MHC subunit chain” as used herein refers to the alpha and beta subunits of MHC molecules. An MHC II molecule is made up of an alpha chain which is constant among each of the DR, DP, and DQ variants and a beta chain which varies by allele. The MHC I molecule is made up of a constant beta macroglobulin and a variable MHC A, B or C chain.

As used here in “virome” comprises the viruses present in a human subject, latently chronically or during acute infection, or a sub-set thereof made up of viruses of a particular taxonomic group or of the viruses located in a particular tissue or organ.

“Immunoglobulinome” as used herein refers to the total complement of immunoglobulins produced and carried by any one subject.

As used herein “allergome” refers to all proteins which may give rise to allergies. This includes proteins recorded in allergen datasets such as that represented at www.allergome.com, http://www.allergenonline.org/, http://comparedatabase.org/www.allergen.org as well as included in Uniprot, Swiss prot, etc.

As used herein the term “repertoire’ is used to describe a collection of molecules or cells making up a functional unit or whole. Thus, as one non limiting example, the entirely of the B cells or T cells in a subject comprise its repertoire of B cells or T cells. The entirety of all immunoglobulins expressed by the B cells are its immunoglobulinome or the repertoire of immunoglobulins. A collection of proteins or cell clonotypes which make up a tissue sample, an individual subject or a microorganism may be referred to as a repertoire.

As used herein “mutated amino acid” refers to the appearance of an amino acid in a protein that is the result of a nucleotide change, a missense mutation, or an insertion or deletion or fusion.

“Splice variant” as used herein refers to different proteins that are expressed from one gene as the result of inclusion or exclusion of particular exons of a gene in the final, processed messenger RNA produced from that gene or that is the result of cutting and re-annealing of RNA or DNA.

“TRAV” as used herein refers to the T cell receptor alpha variable region family or allele subgroups and “TRBV” refers to T cell receptor beta variable region family or allele subgroups as described in IMGT http://imgt.org/IMGTrepertoire/Proteins/index.php #C http://imgt.org/IMGTrepertoire/Proteins/taballeles/human/TRA/TRAV/Hu_TRAVall.html TRAV comprises at least 41 subgroups, with some having sub-subgroups. TRBV comprises at least 30 subgroups. Most combinations of alpha and beta variable region subgroups are encountered. “hTRAV” refers to human TRAV.

As used here in a “receptor bearing cell” is any cell which carries a ligand binding recognition motif on its surface. In some particular instances a receptor bearing cell is a B cell and its surface receptor comprises an immunoglobulin variable region, the immunoglobulin variable region comprising both heavy and light chains which make up the receptor. In other particular instances a receptor bearing cell may be a T cell which bears a receptor made up of both alpha and beta chains or both delta and gamma chains. Other examples of a receptor bearing cell include cells which carry other ligands such as, in one particular non limiting example, a programmed death protein of which there are multiple isoforms.

As used herein the term “bin” refers to a quantitative grouping and a “logarithmic bin” is used to describe a grouping according to the logarithm of the quantity.

As used herein “immunotherapy intervention” is used to describe any deliberate modification of the immune system including but not limited to through the administration of therapeutic drugs or biopharmaceuticals, radiation, T cell therapy, application of engineered T cells, which may include T cells linked to cytotoxic, chemotherapeutic or radiosensitive moieties, checkpoint inhibitor administration, cytokine or recombinant cytokine or cytokine enhancer, including but not limited to a IL-15 agonist, microbiome manipulation, vaccination, B or T cell depletion or ablation, or surgical intervention to remove any immune related tissues.

As used herein “immunomodulatory intervention” refers to any medical or nutritional treatment or prophylaxis administered with the intent of changing the immune response or the balance of immune responsive cells. Such an intervention may be delivered parenterally or orally or via inhalation. Such intervention may include, but is not limited to, a vaccine including both prophylactic and therapeutic vaccines, a biopharmaceutical, which may be from the group comprising an immunoglobulin or part thereof, a T cell stimulator, checkpoint inhibitor, or suppressor, an adjuvant, a cytokine, a cytotoxin, receptor binder, an enhancer of NK (natural killer) cells, an interleukin including but not limited to variants of IL15, superagonists, and a nutritional or dietary supplement. The intervention may also include radiation or chemotherapy to ablate a target group of cells. The impact on the immune response may be to stimulate or to down regulate.

“Checkpoint inhibitor” or “checkpoint blockade” as used herein refers to a type of drug that blocks certain proteins made by some types of immune system cells, such as T cells, and some cancer cells. These proteins help keep immune responses in check and can keep T cells from killing cancer cells. When these proteins are blocked, the “brakes” on the immune system are released and T cells are able to kill cancer cells better. Examples of checkpoint proteins found on T cells or cancer cells include, but are not limited to, PD-1/PD-L1 and CTLA-4/B7-1/B7-2.

As used herein the “cluster of differentiation” proteins refers to cell surface molecules providing targets for immunophenotyping of cells. The cluster of differentiation is also known as cluster of designation or classification determinant and may be abbreviated as CD. Examples of CD proteins include those listed at https://www.uniprot.org/docs/cdlist.

As used herein “microbiome” refers to the constellation of commensal microorganisms found within the human or other host body, inhabiting sites such as the gastrointestine, skin the urogenital tract, the oral cavity, the upper respiratory tract. While most frequently referring to bacteria, the microbiome also may include the viruses in these sites, referred to as the “virome”, or commensal fungi.

As used herein “tumor associated mutations” refers to all nucleotide or amino acid mutations detected in a tumor. In some cases the tumor associated mutations are commonly found within many patients with a particular tumor type. In other cases tumor associated mutations may be unique to a specific patient. In other instances different patients may carry different tumor associated mutations are in the same protein. “Pattern” as used herein means a characteristic or consistent distribution of data points.

As used herein a “frequency pattern” is a data set that displays the frequency of TCEMs in a repertoire of proteins from a proteome associated with an individual subject as compared to the frequency of those TCEMs in a reference database. Particular TCEMs, or groups of TCEMs, within the subject's repertoire may occur at the same, lower or higher frequencies than the corresponding TCEMs in the reference database. The frequency pattern allows identification and categorization of unique TCEMs and/or patterns of TCEMs (i.e., unique features of unique TCEM features). The term “frequency pattern” as used herein is also used to describe the distribution of cellular clonotypes within a repertoire of cells from an individual subject, as compared to the frequency of the cellular clonotypes in a reference database. Particular clonotypes, or groups of clonotypes, within the subject's repertoire may occur at the same, lower or higher frequencies than the corresponding cellular clonotypes in the reference database. The frequency pattern allows identification and categorization of unique patterns of clonotypes. In some embodiments, a “frequency class” or “frequency classification” is assigned to a TCEM motif or to a cellular clonotype based on its frequency as described elsewhere herein.

As used herein “clonotype” is a line of cells derived from a committed or fully differentiated progenitor. In the case of T cells and somatic cells other than B cells, a clonotype of cells has a common genotype, i.e. comprises a common nucleotide sequence. Clonotypes with different nucleotide sequences may express a protein of identical amino acid sequence as a result of different codon utilization. Hence multiple genotypes may lead to a shared phenotype among such clonotypes. In B cells, somatic mutation results in a differentiated cell line comprising a nucleotide sequence that expresses antibodies of one isotype and variable region sequence: this is a B cell clonotype.

As used herein “clonotypic diversity” refers to the distribution of the total number of cells in a repertoire among all unique clonotypes in a repertoire. Hence, if a repertoire has 1 million cells, but these comprise 400,000 of clonotype 1 and 600,000 of clonotype 2, the repertoire has a low clonotypic diversity. If the 1 million cells are distributed as 10 each of 100,000 unique clonotypes the repertoire has a high clonotypic diversity.

As used herein “many to one” describes a relationship in which one protein or peptide sequence is encoded be many different synonymous nucleotide sequences.

As used herein “presentome” refers to the peptides bound in MHC and presented on the surface of antigen presented cells. Mass spectroscopy detects some, but not all, peptides which are part of the presentome.

“Neoantigen” as used herein refers to a novel epitope motif or antigen created as the result of introduction of a mutation into an amino acid sequence. Thus, a neoantigen differentiates a wildtype protein from its mutant-bearing tumor protein homolog, when such mutant is presented to T cells or B cells.

“Tumor specific antigen” or “tumor specific epitope” is used herein to designate an epitope or antigen that differentiates a mutated tumor protein from its unmutated wildtype homologue. Thus, a neoantigen is one type of tumor specific antigen.

As used herein “driver” mutations are those which arise very early in tumorogeneis and are causally associated with the early steps of cell dysregulation. Driver mutations are shared by all clonal offspring arising from the initial tumor cells and offer some additional fitness benefit to the clonal line within its microenvironment. In contrast passenger mutations are those somatic mutations which arise during the differentiation of the tumor and which offer no particular benefit of fitness to the cell. Passengers may serve as biomarkers on tumor cells and may enable some immune evasion. Passenger mutations may differ at different time points in its development and among different parts of a tumor or among metastases. “Driver and passenger” are terms largely interchangeable with “trunk and branch” mutations.

“Bespoke peptides” or “bespoke vaccine” as used herein refers to a peptide or neoantigen or a combination of peptides, or nucleic acid encoding peptides, that are tailored or personalized specifically for an individual patient, taking into account that patient's HLA alleles and mutations. A bespoke peptide or bespoke vaccine is also referred to herein as a “personalized peptide”, “personalized peptide vaccine”, “personalized neoepitope vaccine” or “‘personalized vaccine”.

As used herein “TCGA” refers to The Cancer Genome Atlas (https://cancer.gov/about-nci/organization/ccg/research/structural-genomics/tcga)

As used herein a “polyhydrophobic amino acid” refers to a short chain of natural amino acids which are hydrophobic. Examples include, but are not limited to, leucines, isoleucines or tryptophans where these are assembled in multimers of 5-15 repeats of any one such amino acid. As a non-limiting example, a poly leucine comprising 8 leucines would be an example of a polyhydrophobic amino acid.

A “lipid core peptide system”, as used herein, refers to subunit vaccine comprising a lipoamino acid (LAA) moiety which allows the stimulation of immune activity. A combination of T cell stimulating epitopes or T and B cell stimulating epitopes are linked to a LAA. Multiple different constructs can be created with of different spatial orientation or LAA lengths (e.g. C12 2-amino-D,L-dodecanoic acid or C16, 2-amino-D,L-hexadecanoic acid,). When dissolved in a standard phosphate buffer LCP particles form and the particles facilitate uptake by antigen presenting cells. Different LAA chain lengths lead to different particle sizes.

As used herein, the term “cleavage site octomer” refers to the 8 amino acids located four each side of the bond at which a peptidase cleaves an amino acid sequence. Cleavage site octomer is abbreviated as CSO. “Cathepsin cleavage site octomer” is used herein where the peptidase is a cathepsin.

As used herein “compounding pharmacy” has the meaning defined in sections 503A and 503B of the Federal Food, Drug, and Cosmetic Act

As used herein, a “BAM” file is a compressed binary version of a Sequence Alignment File “SAM” file wherein all nucleotides are aligned to a reference genome. A “BAM slice” is a subset of the entire genome defined by genome coordinates. The HLA locus is located on Chromosome 6. In one particular instance a BAM slice is defined to contain just the HLA locus.

“Immunopathology” when used herein describes an abnormality of the immune system. An immunopathology may affect B-cells and their lineage causing qualitative or quantitative changes in the production of immunoglobulins. Immunopathologies may alternatively affect T-cells and result in abnormal T-cell responses. Immunopathologies may also affect the antigen presenting cells. Immunopathologies may be the result of neoplasias of the cells of the immune system. Immunopathology is also used to describe diseases mediated by the immune system such as autoimmune diseases. Representative autoimmune diseases include, but are not limited to rheumatoid arthritis, diabetes type I and type II, Ankylosing Spondylitis, Atopic allergy, Atopic Dermatitis, Autoimmune cardiomyopathy, Autoimmune enteropathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome, Autoimmune progesterone dermatitis, Autoimmune thrombocytopenia purpura, Autoimmune uveitis, Bullous Pemphigoid, Castleman's disease, Celiac disease, Cogan syndrome, Cold agglutinin disease, Crohn's Disease, Dermatomyositis, Eosinophilic fasciitis, Gastrointestinal pemphigoid, Goodpasture's syndrome, Graves' disease, Guillain-Barré syndrome, Anti-ganglioside Hashimoto's encephalitis, Hashimoto's thyroiditis, Systemic Lupus erythematosus, Miller-Fisher syndrome, Mixed Connective Tissue Disease, Myasthenia gravis, Narcolepsy, Pemphigus vulgaris, Polymyositis, Primary biliary cirrhosis, Psoriasis, Psoriatic Arthritis, Relapsing polychondritis, Sjögren's syndrome, Temporal arteritis, Ulcerative Colitis, Vasculitis, and Wegener's granulomatosis. An allergy is a form of immunopathology. An adverse immune response to an exogenous agent such as a biopharmaceutical protein introduced into a subject is a form of immunopathology.

“Antigen presenting cell” as used herein refers to cells which are capable of presentation of peptides to T cells bound to MHC molecules. This includes but is not limited to the so called “professional” antigen presenting cells comprising, but not limited to, dendritic cells, B cells, and macrophages, but also the so-called non-professional antigen presenting cells in other cell types which carry MHC molecules.

“Parenteral” as used herein refers to any direct injection into the body, including but not limited to intradermal, subcutaneous, intramuscular, intraperitoneal and intravenous injection.

“Non parenteral” as used herein refers to delivery per os to any point in the gastrointestinal tract, to the mucosa of the upper and lower respiratory tract, rectal mucosa or genitourinary tract. Topical application to the skin is also non parenteral

“Originating peptide” as used herein refers to a naturally occurring peptide, whether mutated or not, which comprises a T cell exposed motif and an amino acid of interest therein, that is used as the basis for designing a peptide with desired binding affinity for a particular MHC allele.

“Proposed peptide” as used herein refers to the peptide with desired binding affinity for a particular MHC allele which is designed by changing the amino acids not in the T cell exposed motif and then selected from a list of such peptides for potential inclusion in a vaccination regimen.

As used herein, the term “motif” refers to a characteristic sequence of amino acids forming a distinctive pattern, this may also be expressed as an “amino acid motif”. A “pentamer motif” is a combination of five amino acids, either contiguous to each other or separated by one or more other amino acids.

“HUGO” as used herein refers to the Human Genome Organisation Gene Nomenclature Committee at the European Bioinformatics Institute (genenames.org) which assigns a name and an approved gene symbol to each gene. Examples of HUGO gene names included herein are EGFR (Epidermal growth factor receptor), H3.3 or H33 (Histone H3.3), IDH (isocitrate dehydrogenase), BRAF (Serine/threonine-protein kinase B-raf), TP53 (Cellular tumor antigen p53), PTEN (Phosphatidylinositol 3,4,5-trisphosphate 3-phosphatase and dual-specificity protein phosphatase), ERBB2 (Receptor tyrosine-protein kinase erbB-2), PIK3CA (Phosphatidylinositol 4,5-bisphosphate 3-kinase catalytic subunit alpha isoform), and KRAS (GTPase KRas). Other examples which are found in fusion proteins mentioned herein are KIAA1549-BRAF (UPF0606 protein KIAA1549 fused to Serine/threonine-protein kinase B-raf) and EML4-ALK (Echinoderm microtubule-associated protein-like 4 fused to ALK tyrosine kinase receptor).

“EGFRviii” as used herein refers to the common variant #3 of EGFR in which exons 2-7 are deleted.

“Tumor associated antigen” as used herein refers to an antigen found in a protein that is not mutated or changed from a normal sequence in a tumor but which may be expressed on the surface of a tumor cell and may be expressed at higher levels in a tumor.

“Intravenous immunoglobulin” or “IVIG” as used herein refers to a biopharmaceutical product comprising pooled human immunoglobulin derived from the blood of thousands of donors. Despite its name indicating intravenous administration under some circumstances IVIG may be administered by other routes including intramuscular or subcutaneously.

“Microchimerism” as used herein refers to the presence of a small number of cells that originate from another individual and are therefore genetically distinct from the cells of the host individual. Microchimerism can arise during pregnancy due to exchange of maternal cells into the fetus and fetal cells into the mother.

“Non inherited maternal antigens” or “NIMA” as used herein refers to antigens in cells acquired by offspring from their mother during gestation that are not germline antigens. In particular it refers to cells bearing MHC alleles which are not inherited by the offspring from the mother but are found in the offspring as the result of microchimerism

“Inherited paternal antigens” or “IPA” as used herein refers to antigens in cells acquired by a mother from their offspring during gestation that are germline antigens of the offspring acquired from their father. In particular it refers to cells bearing MHC alleles which are inherited by the offspring from their father and which are found in the mother as the result of microchimerism arising during gestation.

“Haplomatch” or “haploidentical” as used herein refers to an allogeneic transplant which matches half the MHC alleles of the recipient. Typically a haplomatch is a family member.

“Pediatric tumor” or “pediatric cancer” as used herein refers to tumors found predominantly in children, adolescents or young adults arising from mutational events in embryonic, fetal or early childhood development or from inherited mutations.

### DESCRIPTION OF THE INVENTION

The success of neoepitope vaccination in cancer relies on multiple facets of the cellular immune system. After identification of tumor mutations consideration is given to the frequency of occurrence in the tumor and whether the mutated protein is transcribed and expressed. Then, on one hand, a peptide encompassing a tumor mutation must be bound with an adequate affinity to an MHC molecule of the subject affected by cancer in order to be presented to potential cognate T cells. The mutation is most preferably located in a position exposed to the T cell receptor and not hidden in a pocket position of the MHC when presented to a T cell receptor to stimulate a tumor specific response. On the other hand, the subject affected by cancer must carry T cell precursors, as naïve or memory cells, which are capable of mounting a cognate response to the epitope. In many cancer patients the T cell repertoire is depleted, through age and immunosenesence, as a result of prior therapeutic intervention, or because the necessary cognate clones are absent, hence allowing tumor progression. In the particular case of pediatric tumors, the initial mutation may have occurred in utero or at an early age leading to tolerance to the mutation.

The present invention therefore addresses two challenges in neoepitope vaccination: firstly, ensuring that a tumor specific epitope is presented to the T cells by antigen presenting cells carrying the MHC alleles of the cancer subject and, secondly, ensuring that precursor T cells are available to respond to the tumor specific mutation. The first is accomplished by modelling the optimal binding of a peptide carrying a tumor specific motif and, while maintaining this motif constant, varying the flanking amino acids to optimize binding, thereby designing a personalized, or bespoke, peptide to target a T cell response to a tumor specific motif. The second is accomplished in the present invention by providing T cells derived from an allele-matched donor, wherein the T cells are stimulated by the peptide neoepitope of interest presented by an antigen presenting cell. In preferred embodiments the donor of allele-matched T cells is a haplomatched donor, or a familial donor sharing multiple matched alleles. In yet further preferred embodiments, the donor shares microchimerism of MHC alleles with the subject. In the present invention, the goal is therefore to narrowly focus the cells transferred from a donor to those T cells which are cognate for the neoepitope peptide of interest in the tumor when the peptide is presented bound to the MHC alleles which are shared between subject and donor. In essence, the goal is to elicit a precisely focused graft versus host reaction in order to provide an epitope-specific attack on the tumor, while avoiding more disseminated adverse reactions. The methods provided are applicable both to adult subjects and to pediatric subjects with solid tumors, but are of particular utility in pediatric cases.

Tumors carry genes in which mutations give rise to changes in function in the proteins expressed from such genes. The mutations may be manifest as missense mutations, in which one amino acid is replaced by another, as deletions and insertions of codons which maintain the amino acid sequence in frame, or as insertions and deletions of nucleotides which result in a frame shift and generate a novel protein sequence or truncation. A further group of changes in proteins expressed by tumor cells arises from fusions of two gene products. Broadly speaking, tumor mutations may be divided into two groups, drivers and passengers. Drivers are those mutations which are directly oncogenic or are oncogenic by virtue of abrogation of a tumor suppressor function. Passengers are the secondary mutations, which arise stochastically as a sequel to the dysregulation caused by the initial oncogenic event. Some oncogenes and suppressors have well characterized “hot spots” in genes which are the location of many common mutations [2], but the majority of mutations identified in a tumor are stochastic events, and each tumor in each patient is characterized by a different set of mutations. Mutated proteins may comprise novel epitopes, or neoepitopes, which are potential targets for both CD8+ cytotoxic T cells and CD4+ T cells. Considerable success has been achieved by vaccination with peptides comprising neoepitopes, or nucleic acid encoding such peptides, to recruit and target T cells to the tumor [3-8]. However, such successes have been limited, and further improvement of neoantigen targeting is needed [9].

The limitations in neoepitope vaccines to date arise from the evolved nature of tumors, in which mutations present at the time of clinical manifestation have been under pressure to select those motifs that evade the immune response which could otherwise eliminate them. A further limitation is the prior immune experience of the subject who is affected by the tumor. Important factors in prior immune experience include the age of the subject, the age at first occurrence of tumor mutations, and any interventions or other events which may have depleted the diversity of their T cell repertoire. Immune evasion also can occur due to the preferential binding of peptides by the MHC molecules in positions which conceal the mutations in pocket positions of the MHC molecules. A further mode of evasion is due to mutations which give rise to very rare amino acid combinations, such that these comprise very unusual T cell exposed motifs. It is less likely that cognate T cells for rare motifs will be found in the T cell repertoire.

The present invention addresses the synthesis of neoepitope peptides comprising tumor specific mutations, wherein the peptides are designed to ensure that the mutated amino acids are exposed to T cell receptors when the peptides are bound by the subject's MHC alleles. In particular embodiments, the invention addresses the utilization of T cells derived from allele-matched donors to compensate for the loss of diversity of the T cell repertoire of the cancer-affected subject, or to compensate for the acquisition of tolerance by the affected subject to the mutations. The donors are matched in at least one MHC allele with the subject, but in most preferred embodiments are haplomatched donors, who share half of their MHC alleles with the affected subject, or who share at least 25% of their alleles. In particular preferred embodiments the donor is a parent, child or sibling of the subject who share multiple alleles with the donor. In yet further preferred embodiments, the donor also shares some degree of microchimerism with the recipient cancer-affected subject.

The present invention embodies methods comprising the collection of peripheral blood monocyte cells (PBMCs) from the selected MHC typed donor, separation of dendritic cells and then contacting of the dendritic cells with neoantigen peptides designed to be optimally bound and presented by the MHC alleles shared by the donor and the cancer-affected subject. The dendritic cells are then contacted with a population of T cells from the donor: and cultured to expand those cognate T cell clones which respond to the MHC-presented peptide. The expanded neoepitope tumor-specific population of T cells are then infused into the cancer-affected subject. In preferred embodiments the T cells responsive to the epitope of interest in culture are separated from the other T cells of different specificities in the population prior to infusion. In some embodiments, the subject may subsequently be vaccinated with the same neoepitope peptides, or with neoepitope peptides sharing the same T cell exposed motif to further expand desired T cell clones. The methods of the present invention may also be combined with other immunotherapies and immunomodulatory interventions administered to the subject, including but not limited to, checkpoint inhibitors and other immune agonists.

### Targeting T Cell Exposed Motifs

In order to stimulate a T cell immune response, two features of the epitope peptide are required. First, the peptide has to be excised from its source protein by endopeptidases and bound by an MHC molecule in an antigen presenting cell, so that it is presented for potential binding with a T cell bearing a T cell receptor. Secondly, the amino acid motif within the peptide must encounter a cognate T cell; wherein the T cell receptor recognizes and binds the exposed amino acid motif presented within the context of the histotope determined by the particular MHC allele. Neoepitope peptides are thus janiform, with two independent functions [10, 11]. The T cell exposed motifs for a CD8+ T cell comprise amino acids 4,5,6,7 and 8, numbered based on amino acids in a 9-mer peptide, and the binding to the MHC is determined by amino acids 1, 2, 3 and 9, referred to as the groove exposed or pocket positions [12]. The T cell exposed motifs for a CD4+ T cell comprise amino acids 2,3,5,7 and 8, based on a 9) mer peptide as the central core of a peptide that is 13-22 amino acids long, and the binding to the MHC is determined by amino acids 1,4,6, and 9 and by the flanking amino acids outside the 9 mer core. In other instances, the T cell exposed motifs for a CD4+ T cell may comprise amino acids −1,3,5,7 and 8, based on a 9 mer peptide as the central core of a peptide that is 13-22 amino acids long, and the binding to the MHC is determined by amino acids 1,2,4,6, and 9, and by the flanking amino acids outside the 9 mer core. Thus, the T cell receptor does not engage those amino acids located in the groove exposed or pocket positions. The amino acids in these groove-exposed or pocket positions may therefore be altered, while maintaining the T cell exposed motif constant, and may be selected to provide a desired binding affinity to a particular MHC allele of interest and to provide a desired degree of binding affinity most likely to result in stimulation of the T cell, rather than non-stimulation or exhaustion. Thus, in the present invention, peptides are designed to stimulate a desired set of T cell clones which will engage a particular T cell exposed motif corresponding to that found in a mutated tumor protein. The cognate T cell clones will therefore also engage the same T cell exposed motif in the mutated tumor peptide when that motif is subsequently encountered in vivo. This approach enables the positioning of the mutated amino acid in one of the exposed positions, such that the T cell receptor and T cell clone or clones stimulated are responsive to a tumor-specific neoepitope. A similar approach I applied to indel and fusion mutations, wherein the unique T cell exposed motifs are identified, and peptides maintaining these exposed motifs designed.

Methods for precisely predicting MHC binding, identifying and analyzing T cell exposed motifs and generating peptides with altered binding affinity are provided in the following co-pending applications, all of which are incorporated herein by reference in their entirety: PCT US2011/029192, PCT US2012/055038, US2014/014523, PCT US2015/039969, PCT US2017/021781, US Publ. No. 20130330335, US Publ. No. 20160132631, US Publ. No. 20170039314, US Publ. No 20170161430, US Publ. No. 20190070255, PCT US2020/037206, U.S. Pat. Nos. 10,706,955, 10,755,801, and US Prov. Appls. 63/122,191, 63/122,192, 63/122,195, and 63/122,196.

### Determination of Tumor Mutations

In some preferred embodiments, mutated proteins in biopsy samples are identified by sequencing the genome, proteome or transcriptome of cells from a biopsy from a subject. The present invention is not limited to any particular method of obtaining sequences of mutated in a biopsy. A variety of sequencing methods are readily available to those of ordinary skill in the art.

In some preferred embodiments, the present invention utilizes nucleic acid sequencing techniques. The nucleic acid sequences are preferably converted in silico to protein sequences from the identification of mutated amino acids and peptides comprising the mutated amino acids.

In some embodiments, the sequencing is Second Generation (a.k.a. Next Generation or Next-Gen), Third Generation (a.k.a. Next-Next-Gen), or Fourth Generation (a.k.a. N3-Gen) sequencing technology including, but not limited to, pyrosequencing, sequencing-by-ligation, single molecule sequencing, sequence-by-synthesis (SBS), semiconductor sequencing. massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety. Those of ordinary skill in the art will recognize that because RNA is less stable in the cell and more prone to nuclease attack experimentally RNA is usually reverse transcribed to DNA before sequencing.

DNA sequencing techniques include fluorescence-based sequencing methodologies (See, e.g., Birren et al., Genome Analysis: Analyzing DNA, 1, Cold Spring Harbor, N.Y.; herein incorporated by reference in its entirety). In some embodiments, the sequencing is automated sequencing. In some embodiments, the sequencing is parallel sequencing of partitioned amplicons (PCT Publication No: WO2006084132 to Kevin McKernan et al., herein incorporated by reference in its entirety). In some embodiments, the sequencing is DNA sequencing by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional examples of sequencing techniques include the Church polony technology (Mitra et al., 2003, Analytical Biochemistry 320, 55-65: Shendure et al., 2005 Science 309, 1728-1732: U.S. Pat. Nos. 6,432,360, 6,485,944, 6,511,803; herein incorporated by reference in their entireties), the 454 picotiter pyrosequencing technology (Margulies et al., 2005 Nature 437, 376-380; US 20050130173: herein incorporated by reference in their entireties), the Solexa single base addition technology (Bennett et al., 2005, Pharmacogenomics, 6, 373-382; U.S. Pat. No. 6,787,308: U.S. Pat. No. 6,833,246; herein incorporated by reference in their entireties), the Lynx massively parallel signature sequencing technology (Brenner et al. (2000). Nat. Biotechnol. 18:630-634; U.S. Pat. Nos. 5,695,934; 5,714,330; herein incorporated by reference in their entireties), and the Adessi PCR colony technology (Adessi et al. (2000). Nucleic Acid Res. 28, E87: WO 00018957; herein incorporated by reference in its entirety).

Next-generation sequencing (NGS) methods share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbiol., 7: 287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), Life Technologies/Ion Torrent, the Solexa platform commercialized by Illumina, GnuBio, and the Supported Oligonucleotide Ligation and Detection (SOLID) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., and Pacific Biosciences, respectively.

In pyrosequencing (Voelkerding et al., Clinical Chem., 55: 641-658, 2009; MacLean et al., Nature Rev. Microbiol., 7: 287-296; U.S. Pat. No. 6,210,891: U.S. Pat. No. 6,258,568; each herein incorporated by reference in its entirety), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 10⁶ sequence reads can be achieved, resulting in up to 500 million base pairs (Mb) of sequence.

In the Solexa/Illumina platform (Voelkerding et al., Clinical Chem., 55: 641-658, 2009; MacLean et al., Nature Rev. Microbiol., 7: 287-296; U.S. Pat. Nos. 6,833,246; 7,115,400; 6,969,488; each herein incorporated by reference in its entirety), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 250 nucleotides, with overall output exceeding 1 billion nucleotide pairs per analytical run.

Sequencing nucleic acid molecules using SOLID technology (Voelkerding et al., Clinical Chem., 55: 641-658, 2009; MacLean et al., Nature Rev. Microbiol., 7: 287-296: U.S. Pat. No. 5,912,148: U.S. Pat. No. 6,130,073; each herein incorporated by reference in their entirety) also involves fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLID system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, sequencing is nanopore sequencing (see, e.g., Astier et al., J. Am. Chem. Soc. 2006 Feb. 8: 128(5): 1705-10, herein incorporated by reference). The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore, this causes a change in the magnitude of the current through the nanopore that is distinct for each of the four bases, thereby allowing the sequence of the DNA molecule to be determined.

In certain embodiments, sequencing is HeliScope by Helicos BioSciences (Voelkerding et al., Clinical Chem., 55: 641-658, 2009; MacLean et al., Nature Rev. Microbiol., 7: 287-296: U.S. Pat. Nos. 7,169,560; 7,282,337; 7,482,120; 7,501,245; 6,818,395; 6,911,345; 7,501,245; each herein incorporated by reference in their entirety). Template DNA is fragmented and polyadenylated at the 3′ end, with the final adenosine bearing a fluorescent label. Denatured polyadenylated template fragments are ligated to poly(dT) oligonucleotides on the surface of a flow cell. Initial physical locations of captured template molecules are recorded by a CCD camera, and then label is cleaved and washed away. Sequencing is achieved by addition of polymerase and serial addition of fluorescently-labeled dNTP reagents. Incorporation events result in fluor signal corresponding to the dNTP, and signal is captured by a CCD camera before each round of dNTP addition. Sequence read length ranges from 25-50 nucleotides, with overall output exceeding 1 billion nucleotide pairs per analytical run.

The Ion Torrent technology is a method of DNA sequencing based on the detection of hydrogen ions that are released during the polymerization of DNA (see, e.g., Science 327(5970): 1190 (2010): U.S. Pat. Appl. Pub. Nos. 20090026082, 20090127589, 20100301398, 20100197507, 20100188073, and 20100137143, incorporated by reference in their entireties for all purposes). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per-base accuracy of the Ion Torrent sequencer is ˜99.6% for 50 base reads, with ˜100 Mb to 100Gb generated per run. The read-length is 100-300 base pairs. The accuracy for homopolymer repeats of 5 repeats in length is ˜98%. The benefits of ion semiconductor sequencing are rapid sequencing speed and low upfront and operating costs.

In some embodiments, sequencing is the technique developed by Stratos Genomics, Inc. and involves the use of Xpandomers. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid. Reporter elements of the Xpandomer are then detected.

Additional details relating to Xpandomer-based approaches are described in, for example, U.S. Pat. Pub No. 20090035777, entitled “High Throughput Nucleic Acid Sequencing by Expansion,” filed Jun. 19, 2008, which is incorporated herein in its entirety. Other emerging single molecule sequencing methods include real-time sequencing by synthesis using a VisiGen platform (Voelkerding et al., Clinical Chem., 55: 641-58, 2009; U.S. Pat. No. 7,329,492: U.S. patent application Ser. No. 11/671,956: U.S. patent application Ser. No. 11/781,166; each herein incorporated by reference in their entirety) in which immobilized, primed DNA template is subjected to strand extension using a fluorescently-modified polymerase and florescent acceptor molecules, resulting in detectible fluorescence resonance energy transfer (FRET) upon nucleotide addition.

In other preferred embodiments, the present invention utilizes protein sequencing techniques. In some embodiments, proteins may be sequenced by Edman degradation. See. e.g., Edman and Begg (1967). “A protein sequenator”. Eur. J. Biochem. 1 (1): 80-91; Alterman and Hunziker (2011) Amino Acid Analysis: Methods and Protocols. Humana Press. ISBN 978-1-61779-444-5. In other embodiments, mass spectrometry techniques are utilized to sequence proteins. See. e.g., Shevchenko et al., (2006) “In-gel digestion for mass spectrometric characterization of proteins and proteomes”. Nature Protocols. 1 (6): 2856-60; Gundry et al., (2009) “Preparation of proteins and peptides for mass spectrometry analysis in a bottom-up proteomics workflow” Current Protocols in Molecular Biology. Chapter 10: Unit10.25.

To identify tumor mutations, DNA extracted from both a tumor biopsy and normal tissue (typically PBMCs) is compared as described in Example 3.

In some preferred embodiments, as a separate process, the normal genome is aligned with the exons in MHC molecules responsible for the differences in peptide binding affinities to define the HLA haplotype for the MHC class I and II alleles. In yet another embodiment, the RNA sequence files are then aligned to the reference genome using the STAR aligner that defines the splice junctions in the sequences and in addition creates a .bam file that can be viewed with a genome browser viewer. In preferred embodiments reads of the RNA sequence files are tallied gene (normalized by sequence length) using magicBLAST to provide a metric of gene expression of all genes in the tumor.

### T Cell Repertoire Determines Response

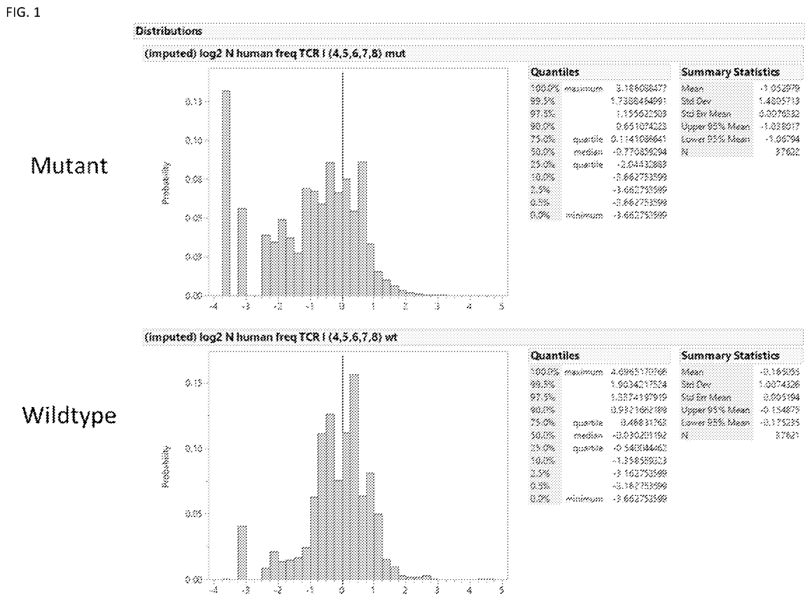

A typical healthy human carries up to 4×10¹⁰ distinct T cell clones [13], and about tenfold higher T cells in toto [14, 15]. Each T cell clone may be responsive to the T cell exposed motifs of a peptide bound in a particular MHC allele, even though peptides comprising that T cell exposed motif (TCEM) may be derived from multiple different sources, and may comprise amino acids outside of the T cell exposed motif and located in the pocket positions which differ. Hence the T cell is polyspecific [16]. The T cell exposed motif comprises 5 exposed amino acids: so there are 205 or 3.2 million possible configurations for CD8+, and likewise for CD4+(potentially twice this number, given two possible configurations of MHC II binding as noted above). Each T cell exposed motif has a determinable frequency distribution in proteomes of interest. For instance, some such T cell exposed motif configurations occur commonly in the human proteome and immunoglobulinome. Other motifs are rarely encountered and yet others are completely absent from the human proteome, but are occasionally found in immunoglobulin variable regions [17, 18]. Analysis of tumor mutations that are present at clinical diagnosis and biopsy shows that in some oncogenes there is a bias towards mutant proteins containing more rare motifs than those that occur in their wild type protein counterparts, as shown in FIG. 1.

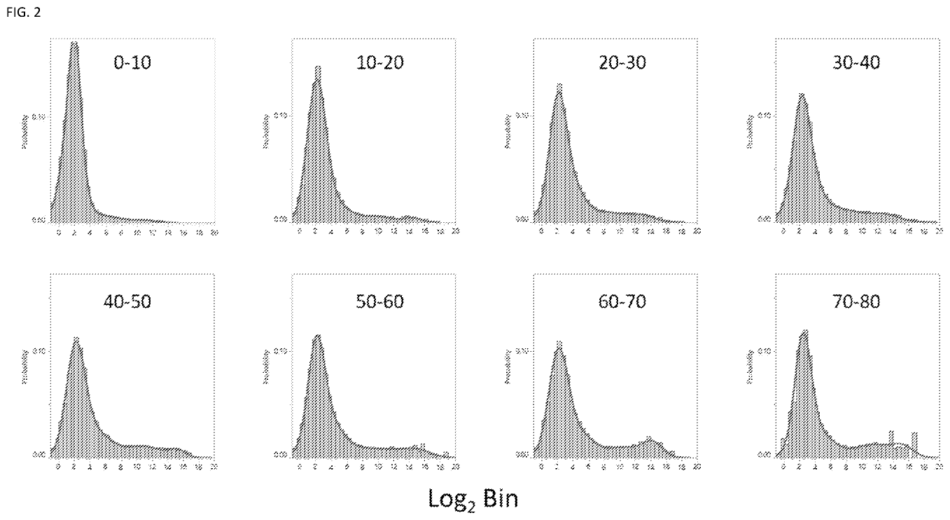

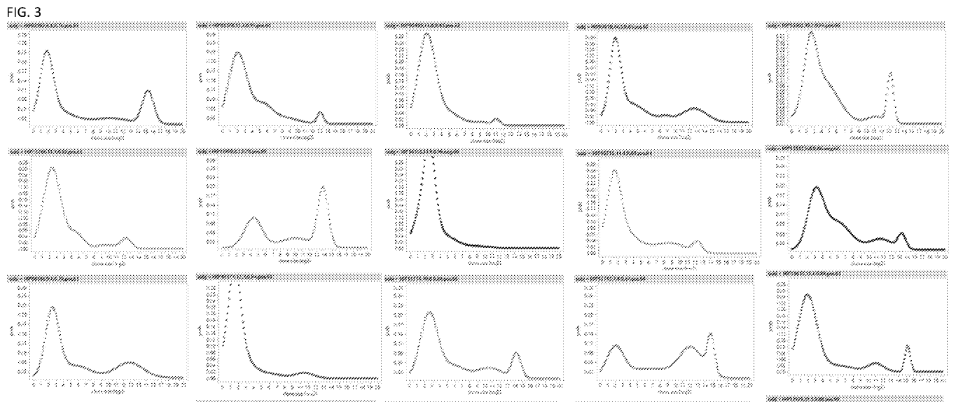

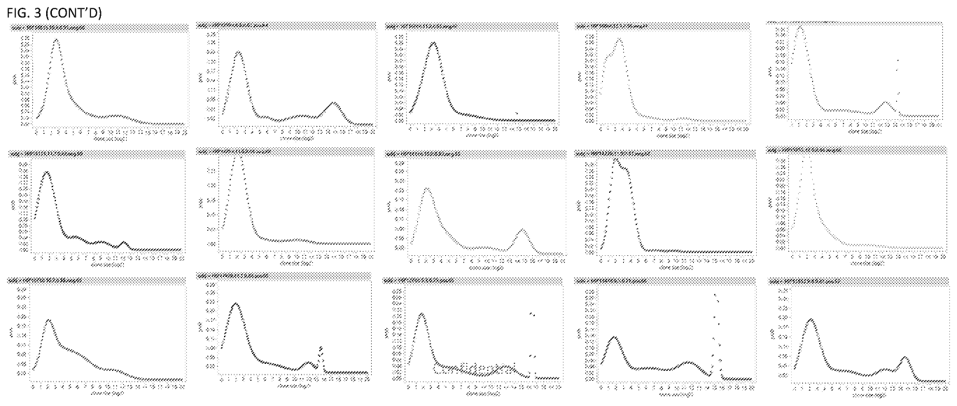

The diversity of clonal populations in the T cell repertoire of an individual is the product of thymic processing and the prior immune exposure of the individual. Thymic processing is most active from birth to about 8 years of age, after which thymic export of new T cells starts to decrease, initially rapidly and then more gradually until about 20 years of age [19]. While some memory T cells have been shown to survive for decades [20], the diversity of residual T cell memory is in constant flux and is a function of restimulation and replenishment of each clone over a lifetime of daily immune stimulation. After early adulthood the repertoire of unique T cell clones carried by each person starts to diminish with age and continues into the immunosenescence of old age [21]. This is depicted in FIG. 2. As shown in FIG. 3 there is considerable individual variation in repertoire diversity, with a few individuals retaining a diversity similar to childhood, while others having a rapid loss of diversity. Infections such as cytomegalovirus may impact repertoire diversity [22]. In cancer patients, interventions from radiation, cytotoxic drugs, and various biotherapeutics may disrupt the normal T cell repertoire. The ability of a subject to respond to a neoepitope vaccine is limited by the presence of naïve precursor or memory T cell clones which are cognate for the tumor specific epitopes that comprise the mutation specific T cell exposed motifs. The more rare a T cell exposed motif, the lesser the chance that a cognate T cell precursor or memory cell is present in the repertoire. Further, it follows that if the tumor cell has escaped immune surveillance and the individual now has a tumor, that affected individual has a higher probability of being deficient in the appropriate cognate T cells. Several approaches to seeking out, identifying and expanding the critical cognate T cell clones have been developed. The development of CAR T technologies has emerged as a workaround for the absence of cognate T cells for tumor cells. The use of autologous dendritic cells and T cells as a means of ex vivo vaccination with neoepitopes or tumor cell lysates has been well described [23-27]. The success of this approach, however, is dependent on there being cognate T cell precursors in the autologous cells sampled from the patient, which for the reasons indicated above may not be the case.

A different set of circumstances may lead to the absence of a tumor epitope-responsive T cell repertoire in the case of pediatric cancers. Here, the tumor mutation has typically arisen in utero, or at a very young age post partum, while thymic processing and tolerization of T cells which bind self-epitopes is still actively in progress [19]. In a small percentage of cases the mutation is inherited. Thus, in these situations, the mutant epitope, even if the mutation is exposed in a T cell exposed motif, is regarded as a self-peptide and no T cell response is mounted to respond to it. Hence, neoepitope vaccination of the child is unlikely to engender any active T cell response to the tumor. Pediatric cancers are typically immunologically “cold” indicating there is no noticeable immune response to the tumor.

The present invention addresses the circumstances where a deficient T cell repertoire limits the ability to respond to a tumor-specific T cell exposed motif. It does so by drawing on the repertoire of an allele-matched donor, and in preferred embodiments a haplomatched related donor, or a donor which shares microchimerism, and then expanding those T cells that respond to a selected tumor specific T cell exposed motif when bound in the MHC allele that is shared with the cancer affected subject. Then, the epitope-specific activated T cells from the allele-matched or haplomatched donor are infused into the cancer affected subject in order to provide an active cytotoxic T cell response targeting the tumor specific epitopes. Applications of this invention include both adult patients, where tumor specific mutations have created rare T cell exposed motifs or where cognate clones are missing or tolerized, and pediatric cancer patients who may have undergone early tolerization to a tumor specific mutation.

Sourcing T Cells from Allele-Matched Donors

The role of MHC alleles in Graft versus Host (GvH) and Host versus Graft (HvG) reactions in tissue transplantation has long been recognized [29, 30]. The difficulty of finding histocompatible donors for tissue transplant has led to development of techniques for using T cell-depleted stem cell transfer and the use of partial matched allogenic donors and haploidentical family members as donors, combined with the use of immunosuppressive drugs. Such an immunosuppressive regimen is counter to the goal of eliciting a T cell response to a tumor neoepitope in a transplant recipient. In most cases the goal is to replace in a cancer patient an immune system which has been ablated by radiation with a healthy immune system “as is” without any modification of the transferred cells.

In the present invention, the goal is to narrowly focus the cells transferred from a donor to those T cells which are cognate for the neoepitope peptide of interest in the tumor when the peptide is presented bound to the MHC alleles which are shared between subject and donor. In essence, the goal is to elicit a graft versus host reaction precisely focused on the tumor mutations in order to provide an epitope specific attack on the tumor while avoiding more disseminated adverse reactions.

There has been considerable success in the transplant of stem cells replete with T cells from haplomatched parent, child and sibling donors which have a degree of microchimerism for non-inherited MHC alleles as long as there is supportive immunosuppressive therapy. There is a surprising degree of microchimerism between parent and child, and between siblings which can increase the acceptability of haplomatched donor cells and mitigate GvH and HvG reactions. In particular, reactivity is reduced to non-inherited maternal antigens (NIMA) to which exposure and transfer occurs during pregnancy, more so than to non-inherited paternal antigens (NIPA). Studies of the persistence of MHC microchimerism have shown approximately 65-70% of mothers carry the paternal MHC acquired from their fetuses and approximately 70% offspring carry the NIMA MHC alleles from their mothers. In both cases such levels of microchimerism are detected through five or six decades of life [32]. Other studies have shown that transplants of non-T cell depleted bone marrow transplants from mothers elicited less GvH disease than transplants from fathers, and that haplomatched siblings with shared non inherited microchimeric alleles had a yet lesser degree of reactivity [33].

In the present invention peptides comprising the tumor mutation specific T cell exposed motif are designed by selection of amino acids in the pocket positions to bind a desired MHC allele, in this case favoring the allele or alleles shared between subject and donor. Such design may also disadvantage binding by the non-shared alleles and favor the stimulation of T cell clones reactive to only the pMHC combination comprising the shared alleles.

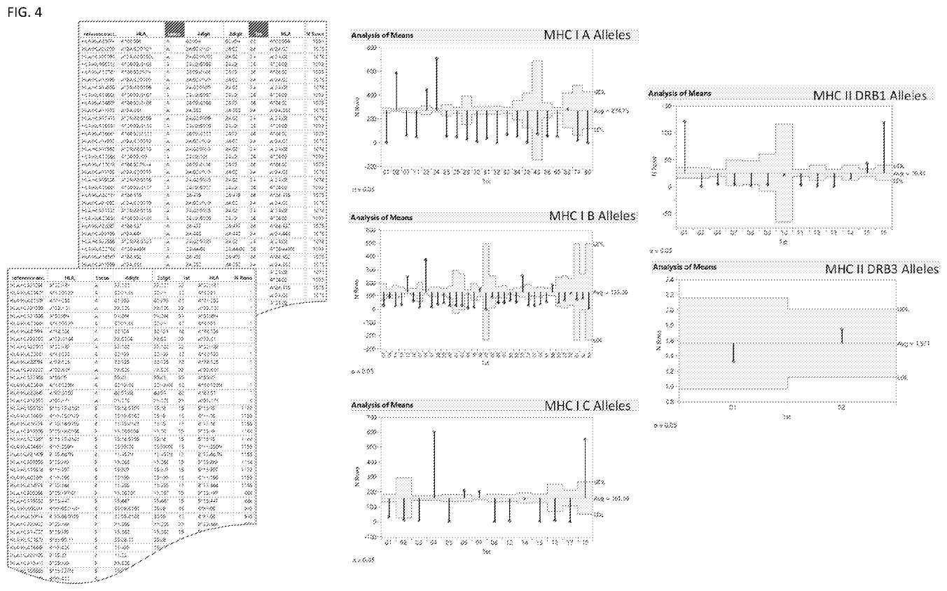

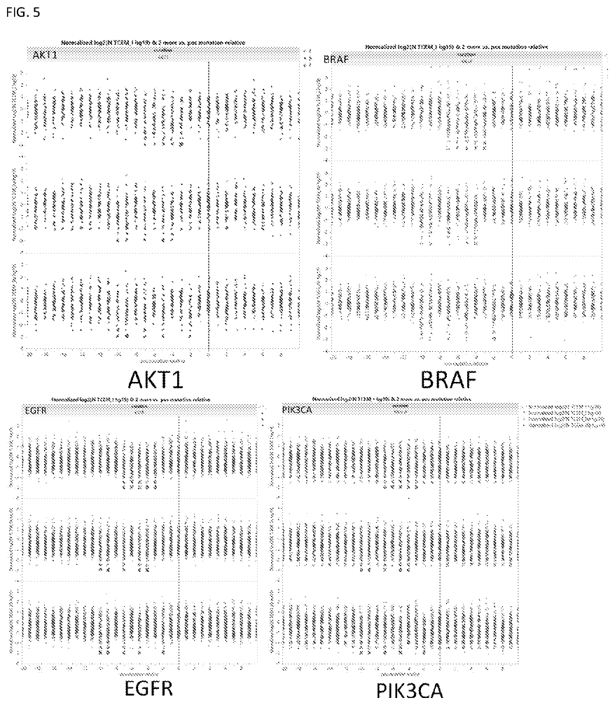

The alleles shared through familial microchimerism, can be detected in the process of determining HLA of a cancer patient through whole exome sequencing at the same time as detection of tumor mutations in a biopsy. In some embodiments this is achieved using an exact aligner based on MagicBLAST [34]. In preferred embodiments this is used with a custom alignment database from the coding DNA sequences of approximately 15,000 human MHC I and MHC II sequences in a repository maintained by IMGT.org. (see the world wide web at ebi.ac.uk/ipd/imgt/hla). Enumeration of 100% matches of whole exome sequences to regions of the gene comprising the genetic differences between alleles. Exomes 2 and 3 of MHC I and exome 2 of MHC II of PBMC are analyzed by Analysis of Means Methods in JMP®. This allows rapid determination of MHC alleles from a tumor biopsy or normal tissue sample. It identifies not only the dominant inherited alleles but also subdominant alleles which are likely indicators of microchimerism. An example of output for one subject is shown in FIG. 4.

### Off-Target T Cell Exposed Motif Matches

Given the polyspecificity of T cell receptor binding, the occurrence of off-target binding of T cells stimulated to respond to a peptide displaying a T cell exposed motif that comprises a tumor-specific mutation is of concern as a source of potential adverse reactions. Therefore, in one embodiment the present invention provides a method to identify potential unintended protein targets in the human proteome and to determine if such potential collateral targets are of concern for the particular subject according to the predicted binding to MHC alleles that subject carries of the peptide carrying that T cell exposed motif in the potential adverse target protein. The application of this embodiment provides a list of the proteins in the human proteome which may be inadvertently targeted by CD8+ or CD4+ T cells stimulated by the peptide arrays selected for T cell targeting of the tumor and with sufficient binding affinity to MHC alleles of the particular subject to stimulate T cells. In one embodiment the list is flagged to identify proteins of particular concern because they have a critical function or are non-redundant and the list is provided to the oncologist to enable an informed risk benefit analysis (See, e.g., PCT/US2020/037206, which is incorporated herein by reference in its entirety.

In a further similar embodiment, the list of potential off target peptide targets is used to assess whether a potential donor could safely be vaccinated with the neoepitope peptide of interest in order to expand the T cell clones of interest prior to collection of the PBMCs for in vitro culture. In this application all alleles that the donor carries are considered, not just those which are shared with the proposed recipient subject.

### In Vitro Stimulation of T Cells

Methods for in vitro maturation of dendritic cells and co-cultivation with T cells have been well described in the context of adoptive cell transfer with autologous T cells and are known to those skilled in the art [35-37]. This has included the induction of neoantigen reactive T cells from donors used as a method to validate binding of neoantigens [38].

Introducing the epitope peptides to antigen presenting cells can be accomplished by several methods. In some embodiments activated dendritic cells are cultivated directly with the peptides of interest. In other embodiments the peptides are incorporated into a particulate format. In some embodiments this is a nanoparticulate. In others the peptide is combined with a lipid to generate liposomes. In further embodiments the peptide is applied as a virus like particle.

In yet other preferred embodiments, the peptide is encoded in a nucleic acid and presented to the dendritic cell as mRNA, or as a DNA plasmid. In yet other embodiments a nucleic acid encoding the desired peptide is introduced to the dendritic cell by electroporation.

In other embodiments the peptide of interest is delivered as a component of a longer synthetic peptide. And in further embodiments the peptide of interest is operationally linked to an immunoglobulin Fc receptor which is preferentially taken up by a dendritic cell. A key factor when the designed peptide is presented as a part of a longer peptide is to ensure that the allele specific flanking amino acids either side of the T cell exposed motif is retained. This may be achieved by the introduction of preferred cleavage sites, for instance comprising arginines. Methods have been developed for the concentration of activated T cells following in vitro stimulation by means of beads or other surfaces coated with antibody to CD137 [39, 40].

### Donor Pretreatment

In order to determine the shared alleles, the prospective T cell donor first provides a sample of PBMCs to allow HLA typing. Having knowledge of all the alleles carried by the prospective donor permits an evaluation of potential adverse matches of designed or bespoke peptides comprising the recipient tumor specific T cell exposed motif. This is conducted as described in Example 4 below. If no significant adverse matches are identified which are an autoimmunity risk based on predicted binding to any of the donor's alleles, consideration may be given to vaccinating the donor with selected peptides designed to comprise neoepitopes prior to collecting the donated dendritic cells and T cells, in order to expand the relevant cognate T cell clones in vivo first before in vitro expansion. In a preferred embodiment the neoepitope vaccination may be conducted 1, 2, 3 or 4 weeks prior to collection of the donor cells and may comprise one or multiple vaccinations.

In yet other embodiments the cell donor may be provided other pre-treatments to boost the diversity of their T cell repertoire in preparation for donation. Such pre-treatments may include, but are not limited to, administration of human IVIG, or the administration of an oral immunostimulant comprising a probiotic, microbiome inoculation or concentrated immunoglobulins. In some preferred instances the concentrated immunoglobulins are concentrated from milk or eggs.

In some embodiments PBMCs collected from the donor for derivation of dendritic cells and T cells are frozen for subsequent neoepitope stimulation in vitro; in alternative embodiments they are separated and placed in culture immediately. In yet other embodiments the epitope specific T cells may be frozen in aliquots after expansion.

### Relevant Characteristics of Pediatric Cancers

Pediatric cancers in which selected subjects are considered potential beneficiaries of the invention described herein are primarily solid tumors. They include, but are not limited to hepatoblastoma, neuroblastoma, rhabdomyosarcoma, Ewing's sarcoma, ependymoma, medulloblastoma, glioma, pilocytic astrocytoma, retinoblastoma, Wilms tumor (nephroblastoma), adrenocortical carcinoma, osteosarcoma, atypical teratoid/rhabdoid tumor, chordoma, and embryonal tumor. However, a subject, whether pediatric or older who is affected by other tumors are also potential beneficiaries, so this list is not considered limiting.

Neuroblastoma is the most common extracranial pediatric solid tumor and has a poor prognosis, meaning that innovative interventions are urgently needed. Neuroblastoma is initiated from mutations occurring early in development in neural crest cells which develop to form the adrenal medulla and sympathetic nerve ganglia [50]. As familial neuroblastoma is rare, the driving mutations are primarily somatic events. MYCN amplification is an indicator of poor prognosis and is the most common aberration, but 40% cases have somatic alterations in known driver genes and about 50% of these being missense variants spread across many different genes potentially constituting tumor-specific motifs that are targetable by T cells. Neuroblastomas can regress spontaneously, suggesting that immunotherapy could be effective. However, they have various additional means of immune evasion including surface expression of gangliosides and sialic acids and have low levels of MHC expression [50, 52, 53]. Dendritic cell vaccines have been attempted using whole tumor lysates, but not with attention to specific epitopes or the likelihood of tolerance [54].

Brain and CNS malignancies are among the most common solid tumors in children, again arguing for a precision immunotherapeutic approach to avoid collateral damage. Medulloblastomas are among the most commonly occurring of this group of pediatric tumors [55] accounting for some 8-10% of pediatric brain tumors. The origin is neuron precursor cells which acquire mutations in one of several pathways. As with other CNS tumors the mutational load is low limiting the number of potential neoepitope targets. As with neuroblastoma, autologous dendritic cells pulsed with tumor lysates have been examined, as well as several different virus mediated oncolytic approaches [55]. These approaches using broad spectrum stimulation teach away from the precision donor T cell approach proposed herein. On the other hand, one study showed that despite low mutational burden CD8+ T cell responses could be induced to a few neoepitopes, indicating that precisely targeting neoepitopes has promise [49].

Pediatric low-grade gliomas, which account for about 30% of childhood brain tumors, are characterized by a high prevalence of KIAA1549-BRAF fusions, BRAF V600E and NF1 mutations [45]. These mutations are present in about two-thirds of cases. Gene rearrangements and fusions had a better prognosis than the less frequent missense variants.

Ewing's sarcoma, the product of an early mutational event in mesenchymal stem cells, chromosome 21 rearrangements give rise to fusions of the Ewing's sarcoma gene EWS and the Friend leukemia insertion FLII generating unique fusions only in the tumor cells [56]. The fusions are in frame and act as an oncogene [57]. The EWS-FLII fusions adopt several isoforms, the most common comprises EWSR11-7 fused to FLII exons 6-10. The low mutational load in EWS is a constraint to the use of checkpoint inhibitors as is the downregulation of HLA [58]. Experimentally the use of a neoepitope vaccine spanning the fusion junction has been shown in mice [59], again indicating promise for a neoepitope vaccine approach if precisely targeted.

### Adult Solid Tumors

Adult solid tumors including, but not limited to, adrenocortical carcinoma, bladder urothelial carcinoma, breast adenocarcinoma, cervical squamous cell carcinoma, cholangiocarcinoma, colon carcinoma, lymphoid neoplasm diffuse large b-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, chronic myelogenous leukemia, brain lower grade glioma, liver hepatocellular carcinoma, lung adenocarcinoma, lung squamous cell carcinoma, mesothelioma, ovarian serous carcinoma, pancreatic adenocarcinoma, pheochromocytoma and paraganglioma, prostate adenocarcinoma, rectal carcinoma, sarcoma, skin cutaneous melanoma, stomach adenocarcinoma, testicular germ cell tumors, thyroid carcinoma, thymoma, uterine corpus endometrial carcinoma, uterine carcinosarcoma, uveal melanoma are suitable applications of the methods described in the present invention.

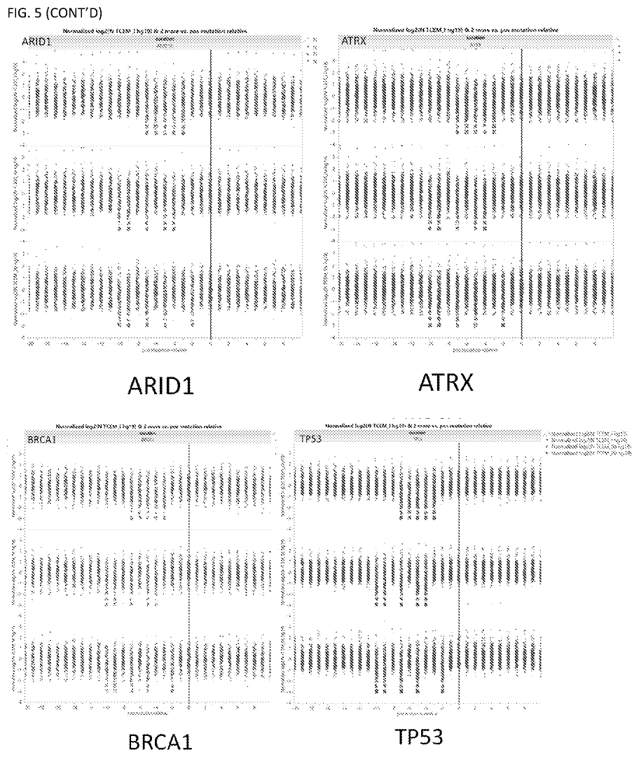

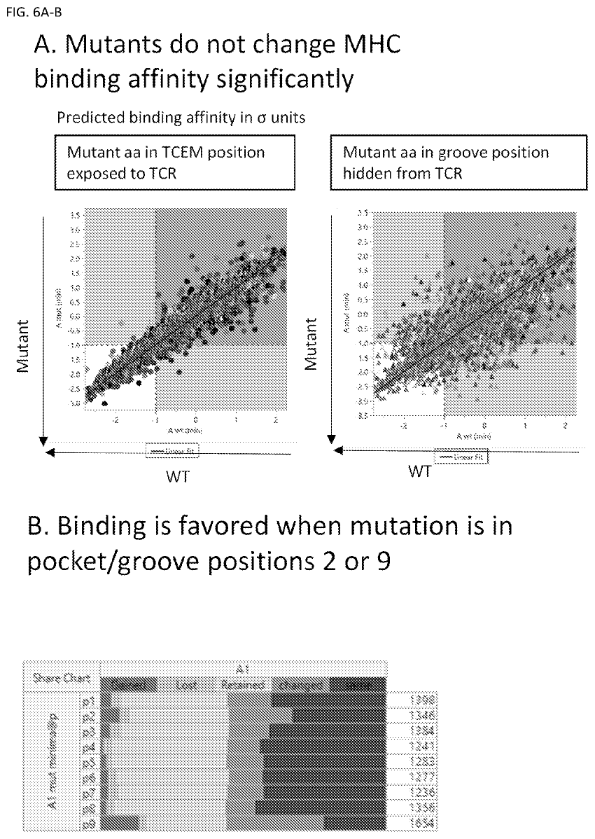

As shown in FIG. 1 examination of 37,622 mutations catalogued in the Genomic Data Commons (see the world wide web at portal.gdc.cancer.gov/) and which are common in 32 types of solid tumor showed that overall these mutations generate T cell exposed motifs which are less likely to be found in the normal human proteome. When the frequency of T cell exposed motifs generated in individual types of cancer were examined a similar pattern was found in each. FIG. 5 shows examples for several common oncogenes and suppressors; this sampling of genes is not considered limiting, as a similar pattern was observed in each of over 120 oncogenes and tumor suppressors. While the overall trend is for T cell exposed motifs in mutants detected at clinical presentation to be less common than their wildtype counterparts, many T cell exposed motifs created by mutation are completely absent from the normal human proteome. Others are rare when compared to the T cell exposed motifs in a representative gastrointestinal microbiome. As the tumor normal T cell exposed motif frequencies are examined for individual cancer patients it is seen that among the personal set of mutations at least some mutants in each case result in rare T cell exposed motifs. The patient is unlikely to have T cells in their extant repertoire that are cognate for such motifs. Furthermore, it was observed that the preferential binding of peptides comprising mutant amino acids was most likely to place the mutant amino acid in a pocket position as seen in FIG. 6. This precludes the exposure of a T cell exposed motif that is tumor specific.

The present invention provides methods to design the flanking regions either side of a potential T cell exposed motif that comprises a mutant amino acid so as to favor binding to the subject's MHC molecules with the mutant amino acid exposed, thus providing a tumor specific epitope and to use allele-matched donor cells to provide the missing repertoire.

### EXAMPLES

### Example 1: Selection of Mutant Peptides and Generation of Better Binding Peptides

The development of vaccines and stimulants for donor dendritic cells and T cells in vitro to comprise peptides with a selected desired affinity for the cancer patient's alleles builds on methods previously described to precisely predict MHC binding, identify and analyze T cell exposed motifs and generate peptides with altered binding affinity (See PCT Appl. US14/41523, PCT Appl. US15/39969, and PCT Appl US17/21781, all of which are incorporated herein by reference in their entirety).

### Identification of Relevant Peptide Positions.

In order for a T cell to differentially target a tumor cell expressing a mutated protein, the mutated amino acid has to be located in a position “visible” or exposed to the T cell receptor and not hidden in the pocket or groove exposed positions that determine binding. We refer to this as the T cell exposed motif or TCEM. A first step in designing a neoepitope vaccine or stimulant panel is therefore to identify those peptide positions which expose the mutated amino acid. For MHC I this means the mutant amino acid must be at positions 4,5,6,7 or 8 of a 9-mer peptide and for MHC II at positions 2,3,5,7,8 of the 9-mer core of a 15 mer. This identifies TCEM IIA; TCEM IIB positions are at −1,3,5,7,8. We first calculate the predicted binding affinity of all sequential peptide positions in the mutant protein and then selected those peptides with relevant TCEM comprising mutated amino acids.

A T cell is only able to target a TCEM in vivo in the context of the tumor if that motif is presented in the host from the naturally occurring mutant peptide. Mutant TCEM that lie in peptides that are extremely unlikely to ever be presented are thus poor targets. We therefore filter the TCEM to identify those which have some likelihood of exposure in the host, limiting to those whose predicted binding affinity is greater than the mean for the protein. This is not an absolute requirement but maximizes the potential for a successful targeting.

For each of the selected peptides comprising a mutant TCEM, a bank of peptides is generated by randomly varying the flanking amino acids, and recalculating the new binding affinity for each allele of interest. For a 9-mer with a pentamer exposed TCEM, this implies up to 160,000 (20+) different peptides could be generated, each with a different binding affinity. For practical purposes a bank of 5,000 or up to 15,000 peptides is usually sufficient to provide peptides within the range of binding affinity desired. For MHC II we opted to vary only those amino acids outside the core 9 mer peptide comprising the TCEM, as the intercalated amino acids which are in pocket (groove exposed) positions affect binding but may also influence the positioning of the exposed amino acids.

A further practical consideration is solubility of the peptide. A score is generated based on the polarity of the constituent amino acids and those peptides most likely to be soluble are put forward as candidates. Sufficient candidate peptides can be generated to prevent this from becoming a limitation.

For a group of 5 proteins each with one mutation and a patient with 4 known alleles therefore a maximum number of allele TCEM combinations is 5 TCEM×5 proteins×4 alleles or 100 possible ways to stimulate T cells which will uniquely target those mutated proteins within that individual. This total number of candidates is down-selected somewhat by removing those in which there is a very low probability of natural presentation.

### Example 2: Selection of Personalized Simulated Peptides

The process described in Example I generates a selection of peptides of different binding affinity for each combination of mutant-containing-TCEM for those patient alleles shared with a prospective T cell donor. Peptides are then selected which have a desired predicted binding affinity for the shared alleles. As peptides of many different binding affinities are provided, the desired MHC binding affinity may be selected for each MHC allele of interest. To achieve stimulation, predicted binding affinity of approximately about 2 standard deviations below the mean of the whole protein is desirable, placing them at about the 95th percentil; i.e. the top 5% binders, but not higher, because conceivably very high affinity peptide could lead to immunosuppression or exhaustion. However, this is a non-limiting example, and the desired affinity may be adjusted based on the particular circumstances. In addition, preference is given to those peptides which also have a low probability of binding for the non-shared alleles carried by the donor but not the recipient subject. Typically this results in selection of a peptide with binding affinity of less than 500 nanomolar, and preferably less than 200 nanomolar or less than 100 nanomolar, or less than 50 nanomolar.

### Example 3: Identification of Tumor Mutations and Screening for Inherited Mutations in the Donor

The following example provides one example of how comparison of DNA sequences between a tumor biopsy and normal tissue is accomplished. This approach is a non-limiting example and other methods are applicable [60]. Sequences derived from both the tumor biopsy and normal tissue (typically PBMCs) are determined. The fastq split-read files of both strands of DNA produced by the sequencing instruments for the normal and tumor DNA and the reverse transcribed RNA are used as input. The DNA sequences are first converted to the alignment map (.bam) files where the paired reads are matched and combined for computational purposes. The sequences are then aligned to the hg38 reference genome using the Burroughs Wheeler aligner. Output of the alignment is then sorted by genome coordinate and the base quality scores recalibrated for these processed files. This recalibration improves the accuracy of the subsequent step where the tumor and normal samples are compared across all genome coordinates. In this step somatic mutations in the tumor that are not found in the normal (germline) produce a.vcf file that defines the variants at specific genome coordinates, and the frequency of the mutations in the tumor cells and the accuracy of the determination based on the sequence characteristics in the region. Once coordinates with mutations are defined it is possible to assign these changes to specific genes and proteins in the genome. The .vcf files are further used to compare the variants at any gene coordinate to a large database of aggregated genomic data from many different ethnic backgrounds. A tumor mutation that is rare will not be found in the database the normal sequences of over a hundred thousand individuals and thus is deemed a truly unique stochastic event in this tumor.

RNA transcripts from the tumor are also determined. Mutant proteins are evaluated to determine if there is RNA transcription indicative of expression. This is taken into consideration as a factor in down selecting apparent mutants to a set of potentially targetable neoepitopes.

Bulk RNA transcript enumeration is carried out using a bioinformatic process that has been designed to tally transcription of different genes. The resulting data is expressed as the FPKM (fragments per kilobase per million total reads) that normalizes the metric for both the length of the transcribed coding region and the number of total reads in the bulk sample detected by the sequencing machine. The bioinformatic software used for transcript enumeration (Magic-BLAST from NCBI) has been designed to assess gene expression and as such is not directly capable of measuring the frequency of potentially mutated codons within the transcripts. In order to compute the mutant frequency in the mRNA transcripts it is necessary to separately enumerate the normal and mutant transcripts. This is achieved by creating a version of the SAM (sequence alignment map) file of the RNA sequences with a bioinformatic software that modifies the cigar (compact idiosyncratic gapped alignment report) strings that map the alignments of the (missing) intronic sequences in the mRNA. Once this modified SAM file is created it can be processed with the standard mutation detection tool, such as mutect 2 that provides the differential mutant and normal read tallies. The ratios of these read tallies are thus the mutant and normal frequency of the allele in the mRNA transcripts. If both parental chromosomes are being expressed equally then the frequency of the mutant and normal allele in the RNA will correlate with the frequency in the DNA. Allele specific differences in expression will give rise to poor correlations. In the extreme, where there is highly differential expression of the parental chromosomes, the mutant may be the only one expressed or may not be expressed at all compared to the normal.

Therefore, in preferred embodiments, the RNA fraction comprising the mutant amino acid is compared to the tumor DNA tumor fraction encoding the gene mutation. In preferred embodiments tumor specific mutations which can be targeted by T cells are selected from those in which the RNA/DNA ratio exceeds 10%. In most preferred embodiments the targetable mutations are selected from those in which the RNA/DNA ratio exceeds 20%.

### Example 4: Determination of HLA Haplotypes Determined from Whole Exome Sequences

A ‘BAM slice’ of the exome file containing the HLA locus (GRch38=chr6:29722700-33143300) was used. The principles outlined for the Optitype [61] which focuses on the read matches to exons 2 and 3 of the MHC molecules was used in conjunction with the magicBLAST aligner [34]. magicBLAST has features that are particularly suited for this type of application. Optitype has been shown to be one of the most accurate methods [62] but only has prediction capabilities for MHC I and thus teaches away from MHC II typing. This general approach was modified as follows to provide MHC II typing also.

The BAM formatted ‘slice’ was converted to a fastq split read format required by magicBLAST using tools from GATK (Broad Institute). A special magicBLAST database for both MHC I and MHC II needed for the alignment process was created from the IMGT HLA sequence database (imgt.org). Exons 2 and 3 are each 270 nucleotides and code for the amino acid variations that form the basis of the different HLA haplotypes. A matrix 540× N (N=number of reads) was created and was used to tally the 100% read match at each nucleotide position produced by magicBLAST. The magicBLAST 100% alignment statistics in the matrix were then tallied across all reads and matched to the different MHC genotypes. Whereas Optitype uses a special integer linear programming approach with the hit matrix to assign the best fit HLA, we demonstrated that a simple tally of the hits in the matrix are adequate to clearly identify the haplotype of the exome data.

### Example 5: Determination of Potential Off-Target Matches in the Proteome

It is necessary to consider both the potential off target matches in the cancer affected subject, and in the case where a donor is vaccinated prior to donation, to consider the possibility of off target adverse effects to the donor. To identify potential off-target effects of the T cells stimulated by the peptides designed to generate targeting of cancer mutations, we compare the T cell exposed motifs (TCEM) with those in the human proteome to identify relevant matches. The entire human proteome, comprising over 88.000 proteins (including all known isoforms of each protein), was pre-analyzed to determine the binding affinity of each peptide in each protein for all MHC alleles. The TCEM comprised in the peptides selected for each cancer patient, selected as described in Example 1 are assembled into a “call list”. The human proteome reference database is searched for all TCEM matching those on the patient call list and a subset of proteins with matching TCEM is assembled. The peptides in this subset which contain the TCEM on the call list are then examined to determine if the TCEM would be likely to be presented in the MHC corresponding to that patient's alleles. If the proteome peptide comprising the TCEM of interest is predicted to bind to any one of the patient's known alleles with an affinity<1 SD below the mean for the protein, the protein is included in an advisory list. The list is curated to remove duplicates and references to any protein fragments catalogued in UniProt (www.uniprot.org). Individual proteins may be reviewed in UniProt and elsewhere to determine if there is evidence of pathologies arising from deficiencies or mutations in the protein. Instances in which a protein of immediate concern is targeted are flagged with a “caution” and excluded from the proposed peptides encoded in a vaccine or in vitro cell stimulation. Examples include, but are not limited to, coagulation factors, neurotransmitters, complement, and other proteins with known essential and non-redundant functions. Decision on off-targeting of proteins in the advisory list may be based on a risk-benefit analysis of the patient's condition but access to such a list allows the oncologist to make an informed decision. The most complete typing of a patient's alleles enables a more complete assessment of potential off-targets. Notably, as the relevance of each target will depend on its presentation as a result of the MHC binding of the peptide in which the TCEM occurs, identifying the potential off-target impacts is as personalized as the design of the peptide array for that cancer patient, or in the case of vaccination of the donor to that donor.

### Example 6: Personalized Neoepitope Peptides in Missense Mutations in a Pediatric Case

Case NI is an adolescent glioblastoma in which mutations were noted in TP53, H3F3A and tubulin (TUBE1).

Tables 1 and 2 show the predicted natural binding to the alleles carried by this individual of those peptides which would expose the mutant amino acids in the natural context of the tumor. Three features are notable. Firstly, the TP53 mutation creates several motifs that are rare in the normal human proteome (<3 SD below the mean frequency) and would potentially have a low number of cognate T cells. Not shown is that they were also extremely rare in the immunoglobulinome. Secondly, in H3F3A and TUBE1 there are several positions that have an apparently stimulatory level of predicted binding for MHC I A alleles (less than −1.5 SD units). This may be indicative of tolerization of T cells cognate for this allele-peptide combination. Thirdly, there is very little binding to MHC II alleles of the peptides with the tumor specific mutation exposed.

TABLE 1
Predicted binding to subject's MHC I alleles of peptides which place the mutant
amino acid in an exposed position.

| | | | | | | | | Motif |
|---|---|---|---|---|---|---|---|---|
| | | | | | | | | frequency |
| | | | | | | | | relative |
| | | | | Predicted binding to MHC I alleles | | | | to mean |
| | | | SEQ | in standard deviation units relative | | | | Human |
| | Gene | TCEM I | ID | to the mean for the proteins | | | | proteome |
| gi | symbol | motif | NO: | A_1101 | A_0301 | B_4403 | B_4001 | TCEM I |
| P04637-R273H | TP53 | SFEVH | 1 | 1.07 | 0.21 | −0.03 | −0.26 | −2.04 |
| P04637-R273H | TP53 | FEVHV | 2 | 0.41 | 0.09 | 0.28 | 0.25 | −0.36 |
| P04637-R273H | TP53 | EVHVC | 3 | 0.08 | 0.70 | 0.17 | −1.60 | −3.66 |
| P04637-R273H | TP53 | VHVCA | 4 | 0.28 | −0.29 | −0.73 | 0.59 | −1.36 |
| P04637-R273H | TP53 | HVCAC | 5 | −0.84 | 0.45 | −2.22 | −3.12 | −3.16 |
| P84243-G35R | H3F3A | PSTGR | 6 | 1.27 | 0.31 | 0.94 | 0.15 | 0.01 |
| P84243-G35R | H3F3A | STGRV | 7 | −2.21 | −1.12 | 0.72 | 0.61 | 0.90 |
| P84243-G35R | H3F3A | TGRVK | 8 | −1.87 | −1.37 | 1.48 | 0.05 | −1.54 |
| P84243-G35R | H3F3A | GRVKK | 9 | −0.79 | −0.20 | 0.16 | 1.02 | −0.86 |
| P84243-G35R | H3F3A | RVKKP | 10 | −0.53 | −1.19 | 0.38 | 0.14 | −1.54 |
| Q9UJT0-T233A | TUBE1 | KLGTA | 11 | 0.77 | 0.04 | 0.60 | −0.17 | 0.08 |
| Q9UJT0-T233A | TUBE1 | LGTAV | 12 | −2.22 | −1.69 | 0.72 | 0.57 | 1.10 |
| Q9UJT0-T233A | TUBE1 | GTAVK | 13 | −0.81 | −0.04 | 0.96 | −0.42 | −0.25 |
| Q9UJT0-T233A | TUBE1 | TAVKP | 14 | −2.32 | −2.74 | 1.31 | 0.53 | −0.30 |
| Q9UJT0-T233A | TUBE1 | AVKPK | 15 | 0.38 | 0.66 | 0.17 | −0.19 | −0.30 |

TABLE 2
Predicted binding to subject's MHC II alleles of peptides which place the
mutant amino acid in an exposed position.

| | | | | Predicted binding to | | |
|---|---|---|---|---|---|---|
| | | | | MHC II alleles- | | Motif |
| | | | | standard deviation | | frequency |
| | | | | units relative to the | | relative |
| | | | SEQ | mean for the proteins | | to mean |
| | Gene | TCEM | ID | DRB1_0701 | | human |
| gi | symbol | IIa | NO: | homozygous | DRB3_0101 | TCEM IIa |
| P04637-R273H | TP53 | RN~F~VH | 16 | −1.38 | −1.80 | −2.44 |
| P04637-R273H | TP53 | NS~E~HV | 17 | −0.14 | −0.30 | −0.47 |
| P04637-R273H | TP53 | FE~H~CA | 18 | 0.97 | 0.28 | −3.62 |
| P04637-R273H | TP53 | VH~C~CP | 19 | 0.01 | −0.14 | −3.62 |
| P04637-R273H | TP53 | HV~A~PG | 20 | 0.69 | −0.17 | −0.29 |
| P84243-G35R | H3F3A | SA~S~GR | 21 | 1.68 | 2.08 | 1.21 |
| P84243-G35R | H3F3A | AP~T~RV | 22 | 1.52 | 1.34 | −0.15 |
| P84243-G35R | H3F3A | ST~R~KK | 23 | 0.30 | 0.92 | 0.62 |
| P84243-G35R | H3F3A | GR~K~PH | 24 | 1.20 | 0.37 | −0.35 |
| P84243-G35R | H3F3A | RV~K~HR | 25 | 0.62 | 0.45 | −0.41 |
| P84243-G35R | H3F3A | VK~P~RY | 26 | 1.25 | 0.40 | −0.24 |
| P84243-G35R | H3F3A | KK~H~YR | 27 | 0.50 | −0.48 | −0.77 |
| Q9UJT0-T233A | TUBE1 | SG~L~TA | 28 | 0.92 | 2.15 | 1.52 |
| Q9UJT0-T233A | TUBE1 | GK~G~AV | 29 | 1.59 | 2.92 | 0.49 |
| Q9UJT0-T233A | TUBE1 | LG~A~KP | 30 | 0.73 | 0.99 | 0.16 |
| Q9UJT0-T233A | TUBE1 | TA~K~KS | 31 | 1.55 | 0.77 | −0.41 |
| Q9UJT0-T233A | TUBE1 | AV~P~SL | 32 | 1.20 | 1.77 | 1.25 |

Peptides were then designed as indicated in Examples 1-3 to provide appropriate binding to stimulate the alleles shared between subject and their T cell donor. In cases where an apparently stimulatory binding affinity was detected the natural peptides were used. The designed peptides are shown in Tables 3 and 4

TABLE 3
MHC I binding bespoke peptides

| | | | | TCEM | | | | | | |
|---|---|---|---|---|---|---|---|---|---|---|
| | | Peptide | | SEQ | | | | | | |
| Gene | proposed | SEQ ID | TCEM | ID | | | | | | |
| symbol | peptide | NO: | core | NO: | Source | Allele | A_0301 | A_1101 | B_4001 | B_4403 |
| TP53 | SNFEVHVCK | 33 | EVHVC | 3 | | B4001 | | | −2.01 | |
| TP53 | SGNVHVCAF | 34 | VHVCA | 4 | | B4403 | | | | −2.04 |
| TP53 | FEVHVCACP | 35 | HVCAC | 5 | natural | B4403 | | | | −2.22 |
| TP53 | GSGHVCACR | 36 | HVCAC | 5 | | A1101 | | −2.06 | | |
| H3F3A | HAASTGRVK | 37 | STGRV | 7 | | A1101 | | −2.08 | | |
| H3F3A | PGWSTGRVP | 38 | STGRV | 7 | | A0301 | −2.07 | | | |
| H3F3A | SSWTGRVKP | 39 | TGRVK | 8 | | A1101 | | −2.08 | | |
| H3F3A | FSPRVKKPK | 40 | RVKKP | 10 | | A0301 | −2.02 | | | |
| TUBE1 | PFKLGTAVK | 41 | LGTAV | 12 | | A0301 | −2.01 | | | |
| TUBE1 | SGKLGTAVK | 42 | LGTAV | 12 | natural | A1101 | | −2.22 | | |
| TUBE1 | SASGTAVKP | 43 | GTAVK | 13 | | A1101 | | −2.00 | | |
| TUBE1 | SPQTAVKPK | 44 | TAVKP | 14 | | A1101 | | −2.00 | | |

TABLE 4
MHC II binding bespoke peptides

| | | Peptide | | TCEM | | | |
|---|---|---|---|---|---|---|---|
| Gene | proposed | SEQ ID | TCEM | SEQ | | | |
| symbol | peptide | NO: | core | ID NO: | Allele | DRB1_0701 | DRB3_0101 |
| TP53 | PLGKRNSFEVHWWAL | 45 | RN~F~VH | 16 | DRB1_0701 | −2.03 | |
| TP53 | PALHRNSFEVHWWNN | 46 | RN~F~VH | 17 | DRB3_0101 | | −2.07 |
| TP53 | VLKHNSFEVHVNMLA | 47 | NS~E~HV | 16 | DRB3_0101 | | −2.08 |
| H3F3A | NGLWSTGRVKKWLLG | 48 | ST~R~KK | 23 | DRB1_0701 | −2.04 | |
| H3F3A | NWLMSTGRVKKLHVL | 49 | ST~R~KK | 24 | DRB1_0701 | −1.98 | |
| H3F3A | NLGHGRVKKPHWLLL | 50 | GR~K~PH | 23 | DRB3_0101 | | −2.07 |
| TUBE1 | WGSWSGKLGTASHNL | 51 | SG~L~TA | 28 | DRB1_0701 | −2.05 | |
| TUBE1 | HANLSGKLGTAAMLS | 52 | SG~L~TA | 28 | DRB1_0701 | −2.05 | |
| TUBE1 | WPWALGTAVKPKKLA | 53 | LG~A~KP | 30 | DRB1_0701 | −2.05 | |

### Example 7: Personalized Neoepitopes in an Adult Glioblastoma

Case 33 is an adult glioblastoma case with mutations in TP53, Serpine 1, TSC1, SEC22A, SLC25A13, and Kinestrin (KNSTRN). As shown in Table 5 and 6 several of the mutations create T cell exposed motifs comprising the mutant amino acid which are quite rare compared to the frequency of the same motif in the human proteome as a reference index (<3 SD below the mean frequency). Also there are several positions that have an apparently stimulatory level of predicted binding for MHC I A alleles (less than −1.5 SD units) although 10 apparently there has not been an effective immune response.

TABLE 5
Predicted binding to subject's MHC I alleles of peptides which place the mutant
amino acid in an exposed position.

| | | | | | Predicted binding to MHC I | | | | Motif |
|---|---|---|---|---|---|---|---|---|---|
| | | | | | alleles in | | | | frequency |
| | | | | | standard deviation units relative | | | | relative |
| | | | | SEQ | to the mean for the proteins | | | | to mean |
| gi | Gene ID | pos | TCEM I | ID NO: | A_0101 | A_0201 | B_0702 | B_3501 | freq TCR I |
| P04637-R248Q | TP53 | 241 | GGMNQ | 54 | 0.52 | 0.05 | 0.22 | 0.40 | −1.21 |
| P04637-R248Q | TP53 | 242 | GMNQR | 55 | −0.46 | −0.85 | 0.63 | 0.56 | −3.66 |
| P04637-R248Q | TP53 | 243 | MNQRP | 56 | −1.69 | −0.47 | −1.22 | −1.58 | −3.66 |
| P04637-R248Q | TP53 | 244 | NQRPI | 57 | 0.37 | −0.28 | −1.67 | −1.67 | −1.21 |
| P04637-R248Q | TP53 | 245 | QRPIL | 58 | −0.16 | −1.88 | 0.14 | 1.32 | −3.16 |
| P05121-T237I | SERPINE1 | 230 | YTEFI | 59 | 0.46 | −0.46 | 0.29 | 0.58 | −2.04 |
| P05121-T237I | SERPINE1 | 231 | TEFIT | 60 | −1.42 | −0.94 | 0.30 | −0.57 | −0.47 |
| P05121-T237I | SERPINE1 | 232 | EFITP | 61 | 0.67 | 0.64 | 0.93 | 1.55 | −1.21 |
| P05121-T237I | SERPINE1 | 233 | FITPD | 62 | −1.05 | −0.42 | 0.46 | −0.63 | −3.66 |
| P05121-T237I | SERPINE1 | 234 | ITPDG | 63 | 0.44 | 0.58 | 0.29 | 0.08 | −0.77 |
| Q92574-R908L | TSC1 | 901 | TSQKL | 64 | −0.93 | −1.29 | −1.98 | −0.95 | 0.29 |
| Q92574-R908L | TSC1 | 902 | SQKLI | 65 | −0.42 | −0.40 | −1.03 | −1.11 | −0.86 |
| Q92574-R908L | TSC1 | 903 | QKLIL | 66 | −0.93 | 0.62 | 0.64 | 1.46 | 0.61 |
| Q92574-R908L | TSC1 | 904 | KLILE | 67 | −1.10 | −0.82 | −0.38 | −1.13 | 0.29 |
| Q92574-R908L | TSC1 | 905 | LILEL | 68 | 1.09 | 0.36 | 2.16 | 2.14 | 1.07 |
| Q96IW7- | SEC22A | 245 | YTGWG | 69 | −0.35 | 0.13 | −0.47 | −0.65 | −3.66 |
| R252G | | | | | | | | | |
| Q96IW7- | SEC22A | 246 | TGWGN | 70 | 1.89 | −0.61 | 0.17 | 1.01 | −1.21 |
| R252G | | | | | | | | | |
| Q96IW7- | SEC22A | 247 | GWGNV | 71 | 0.61 | 0.16 | 1.44 | −0.05 | −1.54 |
| R252G | | | | | | | | | |
| Q96IW7- | SEC22A | 248 | WGNVK | 72 | −0.62 | 0.69 | −0.75 | −0.44 | −0.41 |
| R252G | | | | | | | | | |
| Q96IW7- | SEC22A | 249 | GNVKS | 73 | 0.70 | 0.33 | −1.20 | −1.77 | −0.30 |
| R252G | | | | | | | | | |
| Q9UJS0- | SLC25A13 | 613 | PMGSK | 74 | 0.33 | 0.21 | 0.11 | 1.08 | −0.36 |
| E620K | | | | | | | | | |
| Q9UJS0- | SLC25A13 | 614 | MGSKP | 75 | 1.86 | −0.26 | 1.59 | 0.44 | −2.04 |
| E620K | | | | | | | | | |
| Q9UJS0- | SLC25A13 | 615 | GSKPV | 76 | 0.70 | 0.35 | −1.85 | −0.50 | 0.61 |
| E620K | | | | | | | | | |
| Q9UJS0- | SLC25A13 | 616 | SKPVP | 77 | −0.64 | 0.08 | 0.22 | 1.69 | 0.79 |
| E620K | | | | | | | | | |
| Q9UJS0- | SLC25A13 | 617 | KPVPK | 78 | −0.93 | 0.35 | −0.12 | 0.00 | 0.01 |
| E620K | | | | | | | | | |
| Q9Y448- | KNSTRN | 207 | LEKFW | 79 | −0.57 | 0.37 | 0.47 | 0.94 | −0.86 |
| R214W | | | | | | | | | |
| Q9Y448- | KNSTRN | 208 | EKFWD | 80 | 0.47 | −0.88 | 1.60 | 0.14 | −3.16 |
| R214W | | | | | | | | | |
| Q9Y448- | KNSTRN | 209 | KFWDN | 81 | −0.05 | −2.21 | 0.33 | −0.83 | −3.66 |
| R214W | | | | | | | | | |
| Q9Y448- | KNSTRN | 210 | FWDNC | 82 | 0.34 | 0.09 | 0.07 | −0.53 | −3.66 |
| R214W | | | | | | | | | |
| Q9Y448- | KNSTRN | 211 | WDNCL | 83 | 0.42 | 0.96 | −0.62 | 0.33 | −3.16 |
| R214W | | | | | | | | | |

TABLE 6
Predicted binding to subject's MHC II alleles of peptides which place the mutant
amino acid in an exposed position.

| | | | | | | | Motif |
|---|---|---|---|---|---|---|---|
| | | | | | Predicted binding to MHC II | | frequency |
| | | | | | alleles-standard deviation | | relative to |
| | | | | | units relative | | mean |
| | | | | SEQ ID | to the mean for the proteins | | freq TCR |
| gi | Gene ID | pos | TCEM IIa | NO: | DRB1_1101 | DRB1_1501 | IIa |
| P04637-R248Q | TP53 | 238 | CM~G~NQ | 84 | 1.65 | 0.81 | −3.62 |
| P04637-R248Q | TP53 | 239 | MG~M~QR | 85 | −0.08 | −0.36 | −2.44 |
| P04637-R248Q | TP53 | 241 | GM~Q~PI | 86 | 0.29 | −0.56 | −3.62 |
| P04637-R248Q | TP53 | 242 | MN~R~IL | 87 | 0.47 | −0.40 | 0.58 |
| P04637-R248Q | TP53 | 243 | NQ~P~LT | 88 | −0.62 | −1.55 | −0.41 |
| P04637-R248Q | TP53 | 244 | QR~I~TI | 89 | −1.27 | −1.49 | −1.08 |
| P04637-R248Q | TP53 | 245 | RP~L~II | 90 | −1.28 | −1.71 | 0.64 |
| P05121-T237I | SERPINE1 | 227 | FN~T~FI | 91 | −0.59 | −0.43 | −0.19 |
| P05121-T237I | SERPINE1 | 228 | NY~E~IT | 92 | 0.04 | −0.03 | −2.44 |
| P05121-T237I | SERPINE1 | 230 | TE~I~PD | 93 | −1.76 | −1.28 | −3.62 |
| P05121-T237I | SERPINE1 | 231 | EF~T~DG | 94 | −1.05 | −0.17 | −0.61 |
| P05121-T237I | SERPINE1 | 232 | FI~P~GH | 95 | −0.86 | −1.11 | 0.99 |
| P05121-T237I | SERPINE1 | 233 | IT~D~HY | 96 | −0.62 | −0.53 | −3.62 |
| P05121-T237I | SERPINE1 | 234 | TP~G~YY | 97 | 0.23 | 0.08 | −0.86 |
| Q92574-R908L | TSC1 | 898 | LD~S~KL | 98 | 0.15 | −0.31 | 0.90 |
| Q92574-R908L | TSC1 | 899 | DT~Q~LI | 99 | −0.30 | −0.36 | −0.29 |
| Q92574-R908L | TSC1 | 901 | SQ~L~LE | 100 | 0.05 | −0.53 | 1.49 |
| Q92574-R908L | TSC1 | 902 | QK~I~EL | 101 | 0.11 | 0.21 | 1.41 |
| Q92574-R908L | TSC1 | 903 | KL~L~LE | 102 | 0.20 | −0.61 | 1.73 |
| Q92574-R908L | TSC1 | 904 | LI~E~ES | 103 | −0.07 | −0.61 | 0.89 |
| Q92574-R908L | TSC1 | 905 | IL~L~SH | 104 | −1.02 | −0.77 | 0.09 |
| Q96IW7-R252G | SEC22A | 242 | VY~T~WG | 105 | −1.06 | −0.89 | −3.62 |
| Q96IW7-R252G | SEC22A | 243 | YY~G~GN | 106 | −1.65 | −1.20 | −1.08 |
| Q96IW7-R252G | SEC22A | 245 | TG~G~VK | 107 | −0.71 | −0.93 | −0.54 |
| Q96IW7-R252G | SEC22A | 246 | GW~N~KS | 108 | −0.65 | −0.15 | −2.44 |
| Q96IW7-R252G | SEC22A | 247 | WG~V~SF | 109 | −0.34 | −0.83 | −3.62 |
| Q96IW7-R252G | SEC22A | 248 | GN~K~FL | 110 | 0.50 | −0.06 | −0.69 |
| Q96IW7-R252G | SEC22A | 249 | NV~S~LT | 111 | 0.25 | 0.80 | 0.34 |
| Q9UJS0-E620K | SLC25A13 | 610 | VK~M~SK | 112 | 0.80 | 0.99 | −0.69 |
| Q9UJS0-E620K | SLC25A13 | 611 | KP~G~KP | 113 | 0.08 | 0.02 | −0.06 |
| Q9UJS0-E620K | SLC25A13 | 613 | MG~K~VP | 114 | 0.26 | 0.90 | −3.62 |
| Q9UJS0-E620K | SLC25A13 | 614 | GS~P~PK | 115 | 0.73 | 0.76 | 0.42 |
| Q9UJS0-E620K | SLC25A13 | 615 | SK~V~KS | 116 | 0.18 | −0.08 | 0.42 |
| Q9UJS0-E620K | SLC25A13 | 616 | KP~P~SR | 117 | 0.94 | 0.95 | −0.24 |
| Q9UJS0-E620K | SLC25A13 | 617 | PV~K~RI | 118 | 0.40 | 0.33 | −0.19 |
| Q9Y448-R214W | KNSTRN | 204 | EL~E~FW | 119 | −0.66 | −0.50 | 0.02 |
| Q9Y448-R214W | KNSTRN | 205 | LL~K~WD | 120 | −0.92 | −1.84 | −1.21 |
| Q9Y448-R214W | KNSTRN | 207 | EK~W~NC | 121 | −1.49 | −0.66 | −3.62 |
| Q9Y448-R214W | KNSTRN | 208 | KF~D~CL | 122 | −1.14 | −1.98 | −1.54 |
| Q9Y448-R214W | KNSTRN | 209 | FW~N~LA | 123 | −1.10 | −1.92 | −2.44 |
| Q9Y448-R214W | KNSTRN | 210 | WD~C~AI | 124 | −0.93 | −0.73 | −3.62 |
| Q9Y448-R214W | KNSTRN | 211 | DN~L~IL | 125 | −1.79 | −1.14 | −0.02 |

Therefore, peptides were then designed as indicated in Examples 1-3 to provide appropriate binding to stimulate the alleles shared between the subject and an allele-matched T cell donor. In cases where an apparently stimulatory binding affinity was detected the natural peptides were used. The designed peptides are shown in Tables 7 and 8.

TABLE 7
MHC I binding bespoke peptides

| | | | Peptide | | TCEM | | | | | | |
|---|---|---|---|---|---|---|---|---|---|---|---|
| gene_ | | proposed | SEQ | core | SEQ ID | | | | | | |
| symbol | pos | peptide | ID NO: | TCEM | NO: | Source | Allele | A_0101 | A_0201 | B_0702 | B_3501 |
| KNSTRN | 208 | GVAEKFWDL | 126 | EKFWD | 80 | | A0201 | | −1.99 | | |
| KNSTRN | 209 | LLEKFWDNC | 127 | KFWDN | 81 | natural | A0201 | | −2.21 | | |
| KNSTRN | 211 | SPVWDNCLT | 128 | WDNCL | 83 | | B0702 | | | −2.02 | |
| SEC22A | 245 | ETTYTGWGY | 129 | YTGWG | 69 | | B3501 | | | | −2.02 |
| SEC22A | 246 | KLPTGWGNF | 130 | TGWGN | 70 | | A0201 | | −1.99 | | |
| SEC22A | 248 | HPIWGNVKY | 131 | WGNVK | 72 | | B0702 | | | −2.01 | |
| SERPINE1 | 231 | VANTEFITV | 132 | TEFIT | 60 | | A0201 | | −2.00 | | |
| SERPINE1 | 233 | AGDFITPDA | 133 | FITPD | 62 | | A0101 | −2.06 | | | |
| SERPINE1 | 233 | WPQFITPDV | 134 | FITPD | 62 | | B3501 | | | | −2.02 |
| SLC25A13 | 613 | GVKPMGSKP | 135 | PMGSK | 74 | natural | C0401 | | | | |
| SLC25A13 | 615 | PPGGSKPVG | 136 | GSKPV | 76 | | B0702 | | | −2.08 | |
| SLC25A13 | 617 | SGSKPVPKY | 137 | KPVPK | 78 | | A0101 | −2.06 | | | |
| TP53 | 242 | TLLGMNQRS | 138 | GMNQR | 55 | | A0201 | | −2.05 | | |
| TP53 | 243 | AGEMNQRPI | 139 | MNQRP | 56 | | A0101 | −2.02 | | | |
| TP53 | 243 | MGGMNQRPI | 140 | MNQRP | 56 | natural | A0101 | −1.69 | | | |
| TP53 | 245 | GMNQRPILT | 141 | QRPIL | 58 | natural | A0201 | | −1.88 | | |
| TSC1 | 901 | FSLTSQKLL | 142 | TSQKL | 64 | | B3501 | | | | −2.04 |
| TSC1 | 902 | LPSSQKLIL | 143 | SQKLI | 65 | | B3501 | | | | −2.03 |
| TSC1 | 904 | LGKKLILEP | 144 | KLILE | 67 | | A0101 | −2.05 | | | |

TABLE 8
MHC II binding bespoke peptides

| | | | Peptide | | TCEM | | | | |
|---|---|---|---|---|---|---|---|---|---|
| Gene | | | SEQ ID | TCEM | SEQ ID | | | DRB1_ | DRB1_ |
| symbol | pos | proposed peptide | NO: | core | NO: | Source | Allele | 1101 | 1501 |
| KNSTRN | 207 | SVVIEKFWDNCASYV | 147 | EK~W~NC | 121 | | DRB1_1101 | −2.07 | |
| KNSTRN | 209 | LLEKFWDNCLAILES | 146 | FW~N~LA | 123 | natural | DRB1_1501 | | −1.92 |
| KNSTRN | 210 | LEKFWDNCLAILESK | 145 | WD~C~AI | 124 | natural | DRB3_0202 | | |
| SERPINE1 | 230 | DPVYTEFITPDWLRY | 151 | TE~I~PD | 93 | | DRB1_1101 | −2.02 | |
| SERPINE1 | 230 | RAQYTEFITPDIPPR | 153 | TE~I~PD | 93 | | DRB1_1501 | | 2.06 |
| SERPINE1 | 233 | FTQWITPDGHYKVKL | 152 | IT~D~HY | 96 | | DRB3_0202 | | |
| TP53 | 239 | SSLFMGGMNQRQIWR | 158 | MG~M~QR | 85 | | DRB3_0202 | | |
| TP53 | 241 | QVVFGMNQRPIPWPP | 157 | GM~Q~PI | 86 | | DRB1_1501 | | −2.00 |
| TP53 | 243 | PFAFNQRPILTLPKE | 159 | NQ~P~LT | 88 | | DRB1_1501 | | −2.03 |
| SEC22A | 242 | VILTVYYTGWGWRSR | 148 | VY~T~WG | 105 | | DRB1_1101 | −2.08 | |
| SEC22A | 242 | SILLVYYTGWGWRDS | 150 | VY~T~WG | 105 | | DRB3_0202 | | |
| SEC22A | 247 | RLQLWGNVKSFGFQQ | 149 | WG~V~SF | 109 | | DRB1_1501 | | −2.02 |
| SLC25A13 | 613 | KGFYMGSKPVPPRRR | 154 | MG~K~VP | 114 | | DRB3_0202 | | |
| TSC1 | 903 | RQISKLILELEVYYP | 160 | KL~L~LE | 102 | | DRB1_1501 | | −2.04 |
| TSC1 | 904 | GLWELILELESNFQA | 161 | LI~E~ES | 103 | | DRB1_1501 | | −2.01 |

### Example 8: Personalized Neoepitope Peptides to Stimulate Response to Low Grade Pediatric Glioma Fusion KIAA1549-BRAF

Two mutations are highly characteristic of pediatric low-grade gliomas: KIAA1549-BRAF fusion and BRAFV600E. In one study these together accounted for 68% of pediatric low-grade gliomas [45]. At present only two forms (long and short) of fusion of KIAA1549 to BRAF are described, providing unique neoepitopes at the fusion junction. The in-frame fusion maintains the kinase activity of BRAF while also truncating the N terminal through which the kinase activity of BRAF is regulated [63]. KIAA1549 has 4 recorded isoforms. Two “short forms” lack the initial 1216 amino acids and these may participate in fusions which occur most commonly at 1749 (exon 16). A second isoform of the long canonical form has a deletion of aa 1867-1882 (region absent in the fusion). The most common fusion site is KIAA1549 ex16: BRAF ex9 (approximately 80% cases), exemplified by Genbank gi 211920461, but fusions of KIAA1549ex16: BRAF ex11 (e.g., gi 211920463) and KIAA1549ex15:BRAFex9 (gi 211920465) are also recorded. In TABLE 9 and 10 we show the novel T cell exposed motifs that characterize these 3 fusion junctions and provide bespoke peptides that will target them.

TABLE 9
Bespoke peptides designed for unique motifs at junction site of KIAA1549-BRAF fusions for MHC I alleles

| | | | | | | | | | predicted | predicted | |
|---|---|---|---|---|---|---|---|---|---|---|---|
| | | | | | | | | | affinity | affinity | |
| | | | proposed | SEQ | originating | SEQ ID | TCEM | SEQ ID | proposed | originating | |
| gi | pos | curation | peptide | ID NO: | peptide | NO: | core | NO: | peptide | peptide | Allele |
| 211920461 | 1743 | KIAA1549_BRAF 16_9 | GLPNPCSDL | 185 | TANNPCSDL | 174 | NPCSD | 163 | −2.03 | −0.12 | A0201 |
| 211920461 | 1743 | KIAA1549_BRAF 16_9 | FAVNPCSDV | 186 | TANNPCSDL | 174 | NPCSD | 163 | −2.02 | −0.12 | |
| 211920461 | 1744 | KIAA1549_BRAF 16_9 | NIYPCSDLV | 187 | ANNPCSDLI | 175 | PCSDL | 164 | −2.02 | −0.08 | |
| 211920461 | 1744 | KIAA1549_BRAF 16_9 | VMQPCSDLL | 188 | ANNPCSDLI | 175 | PCSDL | 164 | −2.02 | −0.08 | |
| 211920461 | 1745 | KIAA1549_BRAF 16_9 | IDTCSDLIK | 189 | NNPCSDLIR | 176 | CSDLI | 165 | −2.01 | −0.74 | A0301 |
| 211920461 | 1745 | KIAA1549_BRAF 16_9 | EVRCSDLIP | 190 | NNPCSDLIR | 176 | CSDLI | 165 | −2.02 | −0.74 | |
| 211920461 | 1745 | KIAA1549_BRAF 16_9 | RAECSDLIP | 191 | NNPCSDLIR | 176 | CSDLI | 165 | −2.07 | −1.68 | A1101 |
| 211920461 | 1745 | KIAA1549_BRAF 16_9 | GSNCSDLIP | 192 | NNPCSDLIR | 176 | CSDLI | 165 | −2.03 | −1.68 | |
| 211920461 | 1746 | KIAA1549_BRAF 16_9 | IVASDLIRY | 193 | NPCSDLIRD | 177 | SDLIR | 166 | −2.08 | −0.79 | |
| 211920461 | 1746 | KIAA1549_BRAF 16_9 | SIQSDLIRP | 194 | NPCSDLIRD | 177 | SDLIR | 166 | −2.01 | −0.79 | |
| 211920461 | 1745 | KIAA1549_BRAF 16_9 | EPRCSDLIR | 195 | NNPCSDLIR | 176 | CSDLI | 165 | −2.07 | −1.68 | A6801 |
| 211920461 | 1745 | KIAA1549_BRAF 16_9 | RAVCSDLIK | 196 | NNPCSDLIR | 176 | CSDLI | 165 | −2.06 | −1.68 | |
| 211920461 | 1746 | KIAA1549_BRAF 16_9 | VGTSDLIRK | 197 | NPCSDLIRD | 177 | SDLIR | 166 | −2.07 | −0.58 | |
| 211920461 | 1746 | KIAA1549_BRAF 16_9 | TVLSDLIRK | 198 | NPCSDLIRD | 177 | SDLIR | 166 | −2.07 | −0.58 | |
| 211920461 | 1743 | KIAA1549_BRAF 16_9 | APPNPCSDW | 199 | TANNPCSDL | 174 | NPCSD | 163 | −2.04 | −0.34 | B0702 |
| 211920461 | 1743 | KIAA1549_BRAF 16_9 | NPLNPCSDA | 200 | TANNPCSDL | 174 | NPCSD | 163 | −2.04 | −0.34 | |
| 211920461 | 1744 | KIAA1549_BRAF 16_9 | PGRPCSDLT | 201 | ANNPCSDLI | 175 | PCSDL | 164 | −2.03 | −0.29 | |
| 211920461 | 1744 | KIAA1549_BRAF 16_9 | KGEPCSDLL | 202 | ANNPCSDLI | 175 | PCSDL | 164 | −2.07 | −0.29 | |
| 211920461 | 1746 | KIAA1549_BRAF 16_9 | KPMSDLIRG | 203 | NPCSDLIRD | 177 | SDLIR | 166 | −2.02 | −0.73 | |
| 211920461 | 1746 | KIAA1549_BRAF 16_9 | KPESDLIRA | 204 | NPCSDLIRD | 177 | SDLIR | 166 | −2.02 | −0.73 | |
| 211920461 | 1743 | KIAA1549_BRAF 16_9 | GARNPCSDV | 205 | TANNPCSDL | 174 | NPCSD | 163 | −2.02 | 0.22 | B0801 |
| 211920461 | 1743 | KIAA1549_BRAF 16_9 | AARNPCSDV | 206 | TANNPCSDI | 174 | NPCSD | 163 | −2.03 | 0.22 | |
| 211920461 | 1744 | KIAA1549_BRAF 16_9 | YSRPCSDLV | 207 | ANNPCSDLI | 175 | PCSDL | 164 | −2.04 | 0.10 | |
| 211920461 | 1744 | KIAA1549_BRAF 16_9 | RTRPCSDLV | 208 | ANNPCSDLI | 175 | PCSDL | 164 | −2.04 | 0.10 | |
| 211920461 | 1745 | KIAA1549_BRAF 16_9 | YTFCSDLIT | 209 | NNPCSDLIR | 176 | CSDLI | 165 | −2.01 | −1.89 | B2705 |
| 211920461 | 1745 | KIAA1549_BRAF 16_9 | RNECSDLIR | 210 | NNPCSDLIR | 176 | CSDLI | 165 | −2.00 | −1.89 | |
| 211920461 | 1746 | KIAA1549_BRAF 16_9 | LGKSDLIRF | 211 | NPCSDLIRD | 177 | SDLIR | 166 | −2.03 | −0.70 | B3501 |
| 211920461 | 1746 | KIAA1549_BRAF 16_9 | RVVSDLIRF | 212 | NPCSDLIRD | 177 | SDLIR | 166 | −2.05 | −0.70 | |
| 211920463 | 1746 | KIAA1549_BRAF 16_11 | LVWSKTLGD | 213 | NPCSKTLGR | 178 | SKTLG | 167 | −2.04 | −1.22 | A0301 |
| 211920463 | 1746 | KIAA1549_BRAF 16_11 | IPLSKTLGR | 214 | NPCSKTLGR | 178 | SKTLG | 167 | −2.01 | −1.22 | |
| 211920463 | 1746 | KIAA1549_BRAF 16_11 | DAFSKTLGR | 215 | NPCSKTLGR | 178 | SKTLG | 167 | −2.04 | −1.58 | A1101 |
| 211920463 | 1746 | KIAA1549_BRAF 16_11 | ASGSKTLGR | 216 | NPCSKTLGR | 178 | SKTLG | 167 | −2.04 | −1.58 | |
| 211920463 | 1746 | KIAA1549_BRAF 16_11 | FSVSKTLGK | 217 | NPCSKTLGR | 178 | SKTLG | 167 | −2.07 | −1.77 | A6801 |
| 211920463 | 1746 | KIAA1549_BRAF 16_11 | VAPSKTLGR | 218 | NPCSKTLGR | 178 | SKTLG | 167 | −2.06 | −1.77 | |
| 211920463 | 1744 | KIAA1549_BRAF 16_11 | FMRPCSKTI | 219 | ANNPCSKTL | 179 | PCSKT | 168 | −2.05 | −0.24 | B0702 |
| 211920463 | 1744 | KIAA1549_BRAF 16_11 | LPIPCSKTP | 220 | ANNPCSKTL | 179 | PCSKT | 168 | −2.06 | −0.24 | |
| 211920463 | 1746 | KIAA1549_BRAF 16_11 | LASSKTLGL | 221 | NPCSKTLGR | 178 | SKTLG | 167 | −2.06 | −1.05 | |
| 211920463 | 1746 | KIAA1549_BRAF 16_11 | AGMSKTLGL | 222 | NPCSKTLGR | 178 | SKTLG | 167 | −2.06 | −1.05 | |
| 211920463 | 1744 | KIAA1549_BRAF 16_11 | VSKPCSKTV | 223 | ANNPCSKTL | 179 | PCSKT | 168 | −2.05 | 0.12 | B0801 |
| 211920463 | 1744 | KIAA1549_BRAF 16_11 | KLRPCSKTL | 224 | ANNPCSKTL | 179 | PCSKT | 168 | −2.06 | 0.12 | |
| 211920463 | 1745 | KIAA1549_BRAF 16_11 | ARSCSKTLS | 225 | NNPCSKTLG | 180 | CSKTL | 169 | −2.02 | −0.61 | B2705 |
| 211920463 | 1745 | KIAA1549_BRAF 16_11 | PQLCSKTLL | 226 | NNPCSKTLG | 180 | CSKTL | 169 | −2.03 | −0.61 | |
| 211920463 | 1746 | KIAA1549_BRAF 16_11 | LKDSKTLGS | 227 | NPCSKTLGR | 178 | SKTLG | 167 | −2.05 | −0.22 | |
| 211920463 | 1746 | KIAA1549_BRAF 16_11 | HNFSKTLGR | 228 | NPCSKTLGR | 178 | SKTLG | 167 | −2.01 | −0.22 | |
| 211920463 | 1746 | KIAA1549_BRAF 16_11 | VITSKTLGY | 229 | NPCSKTLGR | 178 | SKTLG | 167 | −2.03 | −1.86 | B3501 |
| 211920463 | 1746 | KIAA1549_BRAF 16_11 | FKFSKTLGY | 230 | NPCSKTLGR | 178 | SKTLG | 167 | −2.06 | −1.86 | |
| 211920465 | 1637 | KIAA1549_BRAF 15_9 | HMAIGCPDL | 231 | SAYIGCPDL | 181 | IGCPD | 170 | −2.00 | −0.32 | A0201 |
| 211920465 | 1637 | KIAA1549_BRAF 15_9 | KIAIGCPDV | 232 | SAYIGCPDL | 181 | IGCPD | 170 | −2.05 | −0.32 | |
| 211920465 | 1638 | KIAA1549_BRAF 15_9 | RVEGCPDLV | 233 | AYIGCPDLI | 182 | GCPDL | 171 | −2.00 | −1.34 | |
| 211920465 | 1638 | KIAA1549_BRAF 15_9 | YLEGCPDLS | 234 | AYIGCPDLI | 182 | GCPDL | 171 | −2.05 | −1.34 | |
| 211920465 | 1639 | KIAA1549_BRAF 15_9 | TSLCPDLIP | 235 | YIGCPDLIR | 183 | CPDLI | 172 | −2.01 | −1.68 | A0301 |
| 211920465 | 1639 | KIAA1549_BRAF 15_9 | QAQCPDLIP | 236 | YIGCPDLIR | 183 | CPDLI | 172 | −2.06 | −1.68 | |
| 211920465 | 1640 | KIAA1549_BRAF 15_9 | KTSPDLIRY | 237 | IGCPDLIRD | 184 | PDLIR | 173 | −2.01 | −1.07 | |
| 211920465 | 1640 | KIAA1549_BRAF 15_9 | VSAPDLIRP | 238 | IGCPDLIRD | 184 | PDLIR | 173 | −2.03 | −1.07 | |
| 211920465 | 1637 | KIAA1549_BRAF 15_9 | AVVIGCPDP | 239 | SAYIGCPDL | 181 | IGCPD | 170 | −2.06 | −0.35 | A1101 |
| 211920465 | 1637 | KIAA1549_BRAF 15_9 | QNHIGCPDK | 240 | SAYIGCPDL | 181 | IGCPD | 170 | −2.04 | −0.35 | |
| 211920465 | 1639 | KIAA1549_BRAF 15_9 | PEQCPDLIK | 241 | YIGCPDLIR | 183 | CPDLI | 172 | −2.05 | −1.79 | |
| 211920465 | 1639 | KIAA1549_BRAF 15_9 | WSACPDLIP | 242 | YIGCPDLIR | 183 | CPDLI | 172 | −2.02 | −1.79 | |
| 211920465 | 1640 | KIAA1549_BRAF 15_9 | AQSPDLIRR | 243 | IGCPDLIRD | 184 | PDLIR | 173 | −2.01 | −1.22 | |
| 211920465 | 1640 | KIAA1549_BRAF 15_9 | SEYPDLIRR | 244 | IGCPDLIRD | 184 | PDLIR | 173 | −2.07 | −1.22 | |
| 211920465 | 1639 | KIAA1549_BRAF 15_9 | KAVCPDLIR | 245 | YIGCPDLIR | 183 | CPDLI | 172 | −2.05 | −1.83 | A6801 |
| 211920465 | 1639 | KIAA1549_BRAF 15_9 | QPTCPDLIR | 246 | YIGCPDLIR | 183 | CPDLI | 172 | −2.02 | −1.83 | |
| 211920465 | 1640 | KIAA1549_BRAF 15_9 | RPPPDLIRR | 247 | IGCPDLIRD | 184 | PDLIR | 173 | −2.07 | −1.20 | |
| 211920465 | 1640 | KIAA1549_BRAF 15_9 | SSLPDLIRK | 248 | IGCPDLIRD | 184 | PDLIR | 173 | −2.03 | −1.20 | |
| 211920465 | 1637 | KIAA1549_BRAF 15_9 | SPLIGCPDY | 249 | SAYIGCPDL | 181 | IGCPD | 170 | −2.04 | −0.93 | B0702 |
| 211920465 | 1637 | KIAA1549_BRAF 15_9 | RGRIGCPDF | 250 | SAYIGCPDL | 181 | IGCPD | 170 | −2.06 | −0.93 | |
| 211920465 | 1638 | KIAA1549_BRAF 15_9 | HLTGCPDLV | 251 | AYIGCPDLI | 182 | GCPDL | 171 | −2.04 | −0.50 | B0801 |
| 211920465 | 1638 | KIAA1549_BRAF 15_9 | EAKGCPDLV | 252 | AYIGCPDLI | 182 | GCPDL | 171 | −2.04 | −0.50 | |
| 211920465 | 1638 | KIAA1549_BRAF 15_9 | TRRGCPDLL | 253 | AYIGCPDLI | 182 | GCPDL | 171 | −2.01 | −0.43 | B2705 |
| 211920465 | 1638 | KIAA1549_BRAF 15_9 | YKSGCPDLP | 254 | AYIGCPDLI | 182 | GCPDL | 171 | −2.01 | −0.43 | |
| 211920465 | 1637 | KIAA1549_BRAF 15_9 | YVPIGCPDW | 255 | SAYIGCPDL | 181 | IGCPD | 170 | −2.06 | −0.61 | B3501 |
| 211920465 | 1637 | KIAA1549_BRAF 15_9 | QATIGCPDW | 256 | SAYIGCPDL | 181 | IGCPD | 170 | −2.03 | −0.61 | |
| 211920465 | 1638 | KIAA1549_BRAF 15_9 | NAKGCPDLW | 257 | AYIGCPDLI | 182 | GCPDL | 171 | −2.02 | −0.10 | |
| 211920465 | 1638 | KIAA1549_BRAF 15_9 | RLVGCPDLF | 258 | AYIGCPDLI | 182 | GCPDL | 171 | −2.02 | −0.10 | |
| 211920465 | 1639 | KIAA1549_BRAF 15_9 | VPVCPDLIR | 259 | YIGCPDLIR | 183 | CPDLI | 172 | −2.04 | −1.58 | |
| 211920465 | 1639 | KIAA1549_BRAF 15_9 | KLHCPDLIF | 260 | YIGCPDLIR | 183 | CPDLI | 172 | −2.04 | −1.58 | |
| 211920465 | 1640 | KIAA1549_BRAF 15_9 | LPQPDLIRF | 261 | IGCPDLIRD | 184 | PDLIR | 173 | −2.04 | −0.80 | |
| 211920465 | 1640 | KIAA1549_BRAF 15_9 | VASPDLIRF | 262 | IGCPDLIRD | 184 | PDLIR | 173 | −2.03 | −0.80 | |

TABLE 10
Bespoke peptides designed for unique motifs at junction site of KIAA1549-BRAF fusions for MHC II alleles

| | | | | | | | | | predicted | predicted | |
|---|---|---|---|---|---|---|---|---|---|---|---|
| | | | | | | | | | binding | binding | |
| | | | | SEQ | | SEQ | | SEQ | affinity | affinity | |
| | | | | ID | originating | ID | | ID | proposed | originating | |
| gi | pos | curation | proposed peptide | NO: | peptide | NO: | TCEM core | NO: | peptide | peptide | Allele |
| 211920461 | 1743 | KIAA1549_BRAF | IAFTPCSDLIRRTFS | 275 | TANNPCSDLIRDQGF | 269 | PC~D~IR | 263 | −2.01 | −0.20 | DRB1_0301 |
| | | 16_9 | | | | | | | | | |
| 211920461 | 1743 | KIAA1549_BRAF | FGRLPCSDLIRVFDK | 276 | TANNPCSDLIRDQGF | 269 | PC~D~IR | 263 | −2.02 | −0.20 | |
| | | 16_9 | | | | | | | | | |
| 211920461 | 1743 | KIAA1549_BRAF | RPRLPCSDLIRVPHP | 277 | TANNPCSDLIRDQGF | 269 | PC~D~IR | 263 | −2.02 | 0.38 | DRB1_0401 |
| | | 16_9 | | | | | | | | | |
| 211920461 | 1743 | KIAA1549_BRAF | PSILPCSDLIRSTKL | 278 | TANNPCSDLIRDQGF | 269 | PC~D~IR | 263 | −2.02 | 0.38 | |
| | | 16_9 | | | | | | | | | |
| 211920461 | 1745 | KIAA1549_BRAF | RSIISDLIRDQGFVA | 279 | NNPCSDLIRDQGFRG | 270 | SD~I~DQ | 264 | −2.00 | 0.03 | DRB1_1501 |
| | | 16_9 | | | | | | | | | |
| 211920461 | 1745 | KIAA1549_BRAF | GSFLSDLIRDQMPPE | 280 | NNPCSDLIRDQGFRG | 270 | SD~I~DQ | 264 | −2.04 | 0.03 | |
| | | 16_9 | | | | | | | | | |
| 211920463 | 1746 | KIAA1549_BRAF | TVLVKTLGRRDLWQL | 281 | NPCSKTLGRRDSSDD | 271 | KT~G~RD | 265 | −2.03 | −0.17 | DRB1_0301 |
| | | 16_11 | | | | | | | | | |
| 211920463 | 1746 | KIAA1549_BRAF | IAAFKTLGRRDKNRE | 282 | NPCSKTLGRRDSSDD | 271 | KT~G~RD | 265 | −2.06 | −0.17 | |
| | | 16_11 | | | | | | | | | |
| 211920465 | 1637 | KIAA1549_BRAF | ISYFGCPDLIRASLM | 283 | SAYIGCPDLIRDQGF | 272 | GC~D~IR | 266 | −2.01 | 0.27 | DRB1_0101 |
| | | 15_9 | | | | | | | | | |
| 211920465 | 1637 | KIAA1549_BRAF | TRFLGCPDLIRGSLM | 284 | SAYIGCPDLIRDQGF | 272 | GC~D~IR | 266 | −2.01 | 0.27 | |
| | | 15_9 | | | | | | | | | |
| 211920465 | 1638 | KIAA1549_BRAF | HLFICPDLIRDSLLH | 285 | AYIGCPDLIRDQGFR | 273 | CP~L~RD | 267 | −2.00 | 0.27 | |
| | | 15_9 | | | | | | | | | |
| 211920465 | 1638 | KIAA1549_BRAF | AFSWCPDLIRDVSYH | 286 | AYIGCPDLIRDQGFR | 273 | CP~L~RD | 267 | −2.06 | 0.27 | |
| | | 15_9 | | | | | | | | | |
| 211920465 | 1639 | KIAA1549_BRAF | DIYLPDLIRDQVGTS | 287 | YIGCPDLIRDQGFRG | 274 | PD~I~DQ | 268 | −2.04 | 0.08 | |
| | | 15_9 | | | | | | | | | |
| 211920465 | 1639 | KIAA1549_BRAF | IPVLPDLIRDQFSLS | 288 | YIGCPDLIRDQGFRG | 274 | PD~I~DQ | 268 | −2.05 | 0.08 | |
| | | 15_9 | | | | | | | | | |
| 211920465 | 1637 | KIAA1549_BRAF | LDMVGCPDLIRRVGV | 289 | SAYIGCPDLIRDQGF | 272 | GC~D~IR | 266 | −2.01 | −0.49 | DRB1_0301 |
| | | 15_9 | | | | | | | | | |
| 211920465 | 1637 | KIAA1549_BRAF | VHEQGCPDLIRWFLH | 290 | SAYIGCPDLIRDQGF | 272 | GC~D~IR | 266 | −2.05 | −0.49 | |
| | | 15_9 | | | | | | | | | |
| 211920465 | 1638 | KIAA1549_BRAF | SDLICPDLIRDPQQL | 291 | AYIGCPDLIRDQGFR | 273 | CP~L~RD | 267 | −2.04 | −0.37 | |
| | | 15_9 | | | | | | | | | |
| 211920465 | 1638 | KIAA1549_BRAF | VELLCPDLIRDLNNQ | 292 | AYIGCPDLIRDQGFR | 273 | CP~L~RD | 267 | −2.04 | −0.37 | |
| | | 15_9 | | | | | | | | | |
| 211920465 | 1639 | KIAA1549_BRAF | YLILPDLIRDQEQTW | 293 | YIGCPDLIRDQGFRG | 274 | PD~I~DQ | 268 | −2.04 | −0.79 | |
| | | 15_9 | | | | | | | | | |
| 211920465 | 1639 | KIAA1549_BRAF | VYLRPDLIRDQQKFA | 294 | YIGCPDLIRDQGFRG | 274 | PD~I~DQ | 268 | −2.05 | −0.79 | |
| | | 15_9 | | | | | | | | | |
| 211920465 | 1637 | KIAA1549_BRAF | YTWRGCPDLIRPTIF | 295 | SAYIGCPDLIRDQGF | 272 | GC~D~IR | 266 | −2.05 | −0.31 | DRB1_0401 |
| | | 15_9 | | | | | | | | | |
| 211920465 | 1637 | KIAA1549_BRAF | YVVYGCPDLIRPQIV | 296 | SAYIGCPDLIRDQGF | 272 | GC~D~IR | 266 | −2.05 | −0.31 | |
| | | 15_9 | | | | | | | | | |
| 211920465 | 1639 | KIAA1549_BRAF | TTVLPDLIRDQEAAR | 297 | YIGCPDLIRDQGFRG | 274 | PD~I~DQ | 268 | −2.05 | −0.22 | |
| | | 15_9 | | | | | | | | | |
| 211920465 | 1639 | KIAA1549_BRAF | ARLEPDLIRDQLLSS | 298 | YIGCPDLIRDQGFRG | 274 | PD~I~DQ | 268 | −2.03 | −0.22 | |
| | | 15_9 | | | | | | | | | |
| 211920465 | 1637 | KIAA1549_BRAF | QIQVGCPDLIRVLPL | 299 | SAYIGCPDLIRDQGF | 272 | GC~D~IR | 266 | −2.09 | 0.18 | DRB1_1501 |
| | | 15_9 | | | | | | | | | |
| 211920465 | 1637 | KIAA1549_BRAF | TVLFGCPDLIRWNRT | 300 | SAYIGCPDLIRDQGF | 272 | GC~D~IR | 266 | −2.05 | 0.18 | |
| | | 15_9 | | | | | | | | | |
| 211920465 | 1638 | KIAA1549_BRAF | FLKLCPDLIRDPAYP | 301 | AYIGCPDLIRDQGFR | 273 | CP~L~RD | 267 | −2.01 | 0.00 | |
| | | 15_9 | | | | | | | | | |
| 211920465 | 1638 | KIAA1549_BRAF | HPVPCPDLIRDPLWT | 302 | AYIGCPDLIRDQGFR | 273 | CP~L~RD | 267 | −2.05 | 0.00 | |
| | | 15_9 | | | | | | | | | |
| 211920465 | 1639 | KIAA1549_BRAF | RLLHPDLIRDQIVVG | 303 | YIGCPDLIRDQGFRG | 274 | PD~I~DQ | 268 | −2.02 | −1.13 | |
| | | 15_9 | | | | | | | | | |
| 211920465 | 1639 | KIAA1549_BRAF | APEFPDLIRDQYYLA | 304 | YIGCPDLIRDQGFRG | 274 | PD~I~DQ | 268 | −2.02 | −1.13 | |
| | | 15_9 | | | | | | | | | |

### Example 9: Preparation of Donor Cells for Infusion

Methods of separation, maturation, culture and stimulation of dendritic cells and T cells are well known to those skilled in the art. The following methods are provided as a non-limiting example, but other methods and variations may be appropriate.

After collection or following thawing of frozen PBMC, the cells are rested overnight in RPMI/pooled AB serum. The following morning, non-adherent cells are separated from the adherent cells. The adherent cells comprising the dendritic cells are washed and then RPMI/AB serum supplemented with GM-CSF and IL4 (final concentration 100 ng/ml and 50 ng/ml respectively) and cultured for seven days (a minimum of four days) to induce dendritic cell maturation.

The non-adherent cells derived from the PBMCs comprise the T cells. Several alternate approaches may be used to achieve separation of CD8+ and CD4+ T cells. Methods using Miltenyi magnetic beads or Dynal beads are appropriate but non limiting examples. Magnetic Miltenyi beads B cells are removed by adding CD19 microbeads and removing the B cells by passing the cell suspension over the magnet column and collecting the flow through. CD8+ and CD4+ T cells are isolated serially, using the magnetic bead for each marker and passing the cells over the magnet. The flow-through contains the cells not selected at the first stage. Selected cells are flushed from the magnet column by washing and pressure with the reagent supplied by the manufacturer. The flow through is then treated with the other bead and the selected cells are recovered from the magnet. Dynal® bead separation differs from Miltenyi in the manner of cell removal from the bead. The cells that adhere to the large Ab-bound beads are released by incubation with a competitor Ab and/or the passage of time.

Matured dendritic cells are exposed to the peptide of interest, as peptides per se, or peptides in a particulate form which may be, but is not limited to, a liposome or nanoparticle, or alternatively as a plasmid or other nucleotide encoded peptide. After incubation for a desired number of hours the T cells are added to the culture.

T cells are cultured in RPMI supplemented with antibiotics, heparin, HEPES, pyruvate, and pooled human AB serum (10% final). CD8+ T cell cultures are also supplemented with IL2 (10U/ml) at the start and at day three. Cultures are typically re-fed after a week by pelleting and resuspending with the same medium and an equal number of irradiated autologous PBMC. Following culture on week 2 or 3 the T cells are recontacted with peptide pulsed dendritic cells.

Activated T cell isolation/concentration may be achieved by rapid flow cytometry with a wider-bore nozzle and in 20% serum. Collecting the cells directly in serum can help maintain viability. In preferred embodiments beads (e.g. Dynal beads) are used for separation prior to infusion. Alternatively, aliquots of cells may be frozen suspended in a sterile cryoprotectant solution containing 10% DMSO, aliquoted into sterile cryovials and frozen for later use.

### REFERENCES

1. Lefranc M P, Giudicelli V, Ginestoux C, Jabado-Michaloud J, Folch G, Bellahcene F, et al. IMGT, the international ImMunoGeneTics information system. Nucleic acids research. 2009; 37(Database issue):D1006-12. Epub 2008/11/04. doi: 10.1093/nar/gkn838. PubMed PMID: 18978023; PubMed Central PMCID: PMC2686541.
2. Vogelstein B, Papadopoulos N, Velculescu V E, Zhou S, Diaz L A, Jr., Kinzler K W. Cancer genome landscapes. Science. 2013; 339(6127):1546-58. Epub 2013/03/30. doi: 10.1126/science.1235122. PubMed PMID: 23539594; PubMed Central PMCID: PMCPMC3749880.
3. Ott P A, Hu Z, Keskin D B, Shukla S A, Sun J, Bozym D J, et al. An immunogenic personal neoantigen vaccine for patients with melanoma. Nature. 2017; 547(7662):217-21. Epub 2017/07/06. doi: 10.1038/nature22991. PubMed PMID: 28678778; PubMed Central PMCID: PMCPMC5577644.
4. Keskin D B, Anandappa A J, Sun J, Tirosh I, Mathewson N D, Li S, et al. Neoantigen vaccine generates intratumoral T cell responses in phase Ib glioblastoma trial. Nature. 2019; 565(7738):234-9. Epub 2018/12/21. doi: 10.1038/s41586-018-0792-9. PubMed PMID: 30568305.
5. Hilf N, Kuttruff-Coqui S, Frenzel K, Bukur V, Stevanovic S, Gouttefangeas C, et al. Actively personalized vaccination trial for newly diagnosed glioblastoma. Nature. 2019; 565(7738):240-5. Epub 2018/12/21. doi: 10.1038/s41586-018-0810-y. PubMed PMID: 30568303.
6. Fang Y, Mo F, Shou J, Wang H, Luo K, Zhang S, et al. A Pan-cancer Clinical Study of Personalized Neoantigen Vaccine Monotherapy in Treating Patients with Various Types of Advanced Solid Tumors. Clin Cancer Res. 2020; 26(17):4511-20. Epub 2020/05/23. doi: 10.1158/1078-0432.CCR-19-2881. PubMed PMID: 32439700.
7. Li F, Chen C, Ju T, Gao J, Yan J, Wang P, et al. Rapid tumor regression in an Asian lung cancer patient following personalized neo-epitope peptide vaccination. Oncoimmunology. 2016; 5(12):e1238539. Epub 2017/01/27. doi: 10.1080/2162402X.2016.1238539. PubMed PMID: 28123873; PubMed Central PMCID: PMCPMC5214696.
8. Hu Z, Leet D E, Allesoe R L, Oliveira G, Li S, Luoma A M, et al. Personal neoantigen vaccines induce persistent memory T cell responses and epitope spreading in patients with melanoma. Nat Med. 2021. Epub 2021/01/23. doi: 10.1038/s41591-020-01206-4. PubMed PMID: 33479501.
9. Fritsch E F, Burkhardt U E, Hacohen N, Wu C J. Personal Neoantigen Cancer Vaccines: A Road Not Fully Paved. Cancer immunology research. 2020; 8(12): 1465-9. Epub 2020/12/03. doi: 10.1158/2326-6066.CIR-20-0526. PubMed PMID: 33262163; PubMed Central PMCID: PMCPMC7717540.
10. Bremel R D, Homan E J. An integrated approach to epitope analysis II: A system for proteomic-scale prediction of immunological characteristics. ImmunomeRes. 2010; 6(1):8. doi: 1745-7580-6-8 [pii]; 10.1186/1745-7580-6-8 [doi].
11. Bremel R D, Homan E J. An integrated approach to epitope analysis I: Dimensional reduction, visualization and prediction of MHC binding using amino acid principal components and regression approaches. Immunome research. 2010; 6:7. Epub 2010/11/04. doi: 10.1186/1745-7580-6-7. PubMed PMID: 21044289; PubMed Central PMCID: PMC2990731.
12. Rammensee H, Bachmann J, Emmerich N P, Bachor O A, Stevanovic S. SYFPEITHI: database for MHC ligands and peptide motifs. Immunogenetics. 1999; 50(3-4):213-9. doi: 90500213.251 [pii].
13. Lythe G, Callard R E, Hoare R L, Molina-Paris C. How many TCR clonotypes does a body maintain? Journal of theoretical biology. 2016; 389:214-24. Epub 2015/11/08. doi: 10.1016/j.jtbi.2015.10.016. PubMed PMID: 26546971; PubMed Central PMCID: PMCPMC4678146.
14. Jenkins M K, Chu H H, Mclachlan J B, Moon J J. On the composition of the preimmune repertoire of T cells specific for Peptide-major histocompatibility complex ligands. Annu Rev Immunol. 2010; 28:275-94. Epub 2010/03/24. doi: 10.1146/annurev-immunol-030409-101253. PubMed PMID: 20307209.
15. Jenkins M K, Moon J J. The role of naive T cell precursor frequency and recruitment in dictating immune response magnitude. J Immunol. 2012; 188(9):4135-40. doi: 10.4049/jimmunol.1102661. PubMed PMID: 22517866; PubMed Central PMCID: PMC3334329.
16. Wucherpfennig K W, Allen P M, Celada F, Cohen I R, De Boer R, Garcia K C, et al. Polyspecificity of T cell and B cell receptor recognition. Seminars in immunology. 2007; 19(4):216-24. Epub 2007/04/03. doi: 10.1016/j.smim.2007.02.012. PubMed PMID: 17398114; PubMed Central PMCID: PMC2034306.
17. Bremel R D, Homan E J. Frequency Patterns of T-Cell Exposed Amino Acid Motifs in Immunoglobulin Heavy Chain Peptides Presented by MHCs. Frontiers in immunology. 2014; 5:541. doi: 10.3389/fimmu.2014.00541. PubMed PMID: 25389426; PubMed Central PMCID: PMC4211557.
18. Bremel R D, Homan J. Extensive T-cell epitope repertoire sharing among human proteome, gastrointestinal microbiome, and pathogenic bacteria: Implications for the definition of self. Frontiers in immunology. 2015; 6. doi: 10.3389/fimmu.2015.00538.
19. Bains I, Thiebaut R, Yates A J, Callard R. Quantifying thymic export: combining models of naive T cell proliferation and TCR excision circle dynamics gives an explicit measure of thymic output. J Immunol. 2009; 183(7):4329-36. Epub 2009/09/08. doi: 10.4049/jimmunol.0900743. PubMed PMID: 19734223.
20. Mold J E, Reu P, Olin A, Bernard S, Michaelsson J, Rane S, et al. Cell generation dynamics underlying naive T-cell homeostasis in adult humans. PLOS biology. 2019; 17(10):e3000383. Epub 2019/10/30. doi: 10.1371/journal.pbio.3000383. PubMed PMID: 31661488; PubMed Central PMCID: PMCPMC6818757.

21 Murray J M, Kaufmann G R, Hodgkin P D, Lewin S R, Kelleher A D, Davenport M P, et al. Naive T cells are maintained by thymic output in early ages but by proliferation without phenotypic change after age twenty. Immunology and cell biology. 2003; 81(6):487-95. Epub 2003/11/26. doi: 10.1046/j.1440-1711.2003.01191.x. PubMed PMID: 14636246.
22. Emerson R O, DeWitt W S, Vignali M, Gravley J, Hu J K, Osborne E J, et al. Immunosequencing identifies signatures of cytomegalovirus exposure history and HLA-mediated effects on the T cell repertoire. Nat Genet. 2017; 49(5):659-65. Epub 2017/04/04. doi: 10.1038/ng.3822. PubMed PMID: 28369038.
23. Chiang C L, Coukos G, Kandalaft L E. Whole Tumor Antigen Vaccines: Where Are We? Vaccines (Basel). 2015; 3(2):344-72. Epub 2015/09/08. doi: 10.3390/vaccines3020344. PubMed PMID: 26343191; PubMed Central PMCID: PMCPMC4494356.
24. Chiang C L, Kandalaft L E. In vivo cancer vaccination: Which dendritic cells to target and how? Cancer Treat Rev. 2018; 71:88-101. Epub 2018/11/06. doi: 10.1016/j.ctrv.2018.10.012. PubMed PMID: 30390423; PubMed Central PMCID: PMCPMC6295330.
25. Harari A, Graciotti M, Bassani-Sternberg M, Kandalaft L E. Antitumour dendritic cell vaccination in a priming and boosting approach. Nature reviews Drug discovery. 2020; 19(9):635-52. Epub 2020/08/09. doi: 10.1038/s41573-020-0074-8. PubMed PMID: 32764681.
26. Dillman R O, Cornforth A N, McClay E F, Depriest C. Patient-specific dendritic cell vaccines with autologous tumor antigens in 72 patients with metastatic melanoma. Melanoma Manag. 2019; 6(2):MMT20. Epub 2019/08/14. doi: 10.2217/mmt-2018-0010. PubMed PMID: 31406564; PubMed Central PMCID: PMCPMC6688559.
27. Ding Z, Li Q, Zhang R, Xie L, Shu Y, Gao S, et al. Personalized neoantigen pulsed dendritic cell vaccine for advanced lung cancer. Signal Transduct Target Ther. 2021; 6(1):26. Epub 2021/01/22. doi: 10.1038/s41392-020-00448-5. PubMed PMID: 33473101; PubMed Central PMCID: PMCPMC7817684.
28. Casey D L, Cheung N V. Immunotherapy of Pediatric Solid Tumors: Treatments at a Crossroads, with an Emphasis on Antibodies. Cancer immunology research. 2020; 8(2):161-6. Epub 2020/02/06. doi: 10.1158/2326-6066.CIR-19-0692. PubMed PMID: 32015013; PubMed Central PMCID: PMCPMC7058412.
29. Ferrara J L, Levine J E, Reddy P, Holler E. Graft-versus-host disease. Lancet. 2009; 373(9674): 1550-61. Epub 2009/03/14. doi: 10.1016/S0140-6736(09)60237-3. PubMed PMID: 19282026; PubMed Central PMCID: PMCPMC2735047.
30. Welniak L A, Blazar B R, Murphy W J. Immunobiology of allogeneic hematopoietic stem cell transplantation. Annu Rev Immunol. 2007; 25:139-70. Epub 2006/11/30. doi: 10.1146/annurev.immunol.25.022106.141606. PubMed PMID: 17129175.
31 Ichinohe T, Uchiyama T, Shimazaki C, Matsuo K, Tamaki S, Hino M, et al. Feasibility of HLA-haploidentical hematopoietic stem cell transplantation between noninherited maternal antigen (NIMA)-mismatched family members linked with long-term fetomaternal microchimerism. Blood. 2004; 104(12):3821-8. Epub 2004/07/29. doi: 10.1182/blood-2004-03-1212. PubMed PMID: 15280193.
32. Ichinohe T, Teshima T, Matsuoka K, Maruya E, Saji H. Fetal-maternal microchimerism: impact on hematopoietic stem cell transplantation. Curr Opin Immunol. 2005; 17(5):546-52. Epub 2005/08/09. doi: 10.1016/j.coi.2005.07.009. PubMed PMID: 16084712.
33. van Rood J J, Loberiza F R, Jr., Zhang M J, Oudshoorn M, Claas F, Cairo M S, et al. Effect of tolerance to noninherited maternal antigens on the occurrence of graft-versus-host disease after bone marrow transplantation from a parent or an HLA-haploidentical sibling. Blood. 2002; 99(5): 1572-7. Epub 2002/02/28. doi: 10.1182/blood.v99.5.1572. PubMed PMID: 11861270.
34 Boratyn G M, Thierry-Mieg J, Thierry-Mieg D, Busby B, Madden T L. Magic-BLAST, an accurate RNA-seq aligner for long and short reads. BMC Bioinformatics. 2019; 20(1):405. Epub 2019/07/28. doi: 10.1186/s12859-019-2996-x. PubMed PMID: 31345161; PubMed Central PMCID: PMCPMC6659269.
35. Rosenberg S A, Restifo N P, Yang J C, Morgan R A, Dudley M E. Adoptive cell transfer: a clinical path to effective cancer immunotherapy. Nature reviews Cancer. 2008; 8(4):299-308. Epub 2008/03/21. doi: 10.1038/nrc2355. PubMed PMID: 18354418; PubMed Central PMCID: PMCPMC2553205.
36 Kast F, Klein C, Umana P, Gros A, Gasser S. Advances in identification and selection of personalized neoantigen/T-cell pairs for autologous adoptive T cell therapies. Oncoimmunology. 2021; 10(1): 1869389. Epub 2021/02/02. doi: 10.1080/2162402X.2020.1869389. PubMed PMID: 33520408; PubMed Central PMCID: PMCPMC7808433.
37. Nair S, Archer G E, Tedder T F. Isolation and generation of human dendritic cells. Current protocols in immunology/edited by John E Coligan [et al]. 2012; Chapter 7:Unit7 32. doi: 10.1002/0471142735.im0732s99. PubMed PMID: 23129155; PubMed Central PMCID: PMCPMC4559332.
38. Ali M, Foldvari Z, Giannakopoulou E, Boschen M L, Stronen E, Yang W, et al. Induction of neoantigen-reactive T cells from healthy donors. Nature protocols. 2019; 14(6): 1926-43. Epub 2019/05/19. doi: 10.1038/s41596-019-0170-6. PubMed PMID: 31101906.
39 Teschner D, Wenzel G, Distler E, Schnurer E, Theobald M, Neurauter A A, et al. In vitro stimulation and expansion of human tumour-reactive CD8+ cytotoxic T lymphocytes by anti-CD3/CD28/CD137 magnetic beads. Scandinavian journal of immunology. 2011; 74(2): 155-64. Epub 2011/04/27. doi: 10.1111/j.1365-3083.2011.02564.x. PubMed PMID: 21517928.
40. Wolfl M, Kuball J, Ho W Y, Nguyen H, Manley T J, Bleakley M, et al. Activation-induced expression of CD137 permits detection, isolation, and expansion of the full repertoire of CD8+ T cells responding to antigen without requiring knowledge of epitope specificities. Blood. 2007; 110(1):201-10. Epub 2007/03/21. doi: 10.1182/blood-2006-11-056168. PubMed PMID: 17371945; PubMed Central PMCID: PMCPMC1896114.
41 Pui C H, Gajjar A J, Kane J R, Qaddoumi I A, Pappo A S. Challenging issues in pediatric oncology. Nat Rev Clin Oncol. 2011; 8(9):540-9. Epub 2011/06/29. doi: 10.1038/nrclinonc.2011.95. PubMed PMID: 21709698; PubMed Central PMCID: PMCPMC3234106.
42. Downing J R, Wilson R K, Zhang J, Mardis E R, Pui C H, Ding L, et al. The Pediatric Cancer Genome Project. Nat Genet. 2012; 44(6):619-22. Epub 2012/05/30. doi: 10.1038/ng.2287. PubMed PMID: 22641210; PubMed Central PMCID: PMCPMC3619412.
43 Grobner S N, Worst B C, Weischenfeldt J, Buchhalter I, Kleinheinz K, Rudneva V A, et al. The landscape of genomic alterations across childhood cancers. Nature. 2018; 555(7696):321-7. Epub 2018/03/01. doi: 10.1038/nature25480. PubMed PMID: 29489754.
44. Chang T C, Carter R A, Li Y, Li Y, Wang H, Edmonson M N, et al. The neoepitope landscape in pediatric cancers. Genome Med. 2017; 9(1):78. Epub 2017/09/01. doi: 10.1186/s13073-017-0468-3. PubMed PMID: 28854978; PubMed Central PMCID: PMCPMC5577668.
45. Ryall S, Zapotocky M, Fukuoka K, Nobre L, Guerreiro Stucklin A, Bennett J, et al. Integrated Molecular and Clinical Analysis of 1,000 Pediatric Low-Grade Gliomas. Cancer Cell. 2020; 37(4):569-83 e5. Epub 2020/04/15. doi: 10.1016/j.ccell.2020.03.011. PubMed PMID: 32289278; PubMed Central PMCID: PMCPMC7169997.
46. Zhang J, Walsh M F, Wu G, Edmonson M N, Gruber T A, Easton J, et al. Germline Mutations in Predisposition Genes in Pediatric Cancer. The New England journal of medicine. 2015; 373(24):2336-46. Epub 2015/11/19. doi: 10.1056/NEJMoa1508054. PubMed PMID: 26580448; PubMed Central PMCID: PMCPMC4734119.
47. Li-Fraumeni Syndrome [Internet]. NCBI. 2020. Available from: ncbi.nlm.nih.gov/books/NBK532286.
48. Dees N D, Zhang Q, Kandoth C, Wendl M C, Schierding W, Koboldt D C, et al. MuSiC: identifying mutational significance in cancer genomes. Genome Res. 2012; 22(8): 1589-98. Epub 2012/07/05. doi: 10.1101/gr.134635.111. PubMed PMID: 22759861; PubMed Central PMCID: PMCPMC3409272.
49. Blaeschke F, Paul M C, Schuhmann M U, Rabsteyn A, Schroeder C, Casadei N, et al. Low mutational load in pediatric medulloblastoma still translates into neoantigens as targets for specific T-cell immunotherapy. Cytotherapy. 2019; 21(9):973-86. Epub 2019/07/29. doi: 10.1016/j.jcyt.2019.06.009. PubMed PMID: 31351799.
50. Cheung N K, Dyer M A. Neuroblastoma: developmental biology, cancer genomics and immunotherapy. Nature reviews Cancer. 2013; 13(6):397-411. Epub 2013/05/25. doi: 10.1038/nrc3526. PubMed PMID: 23702928; PubMed Central PMCID: PMCPMC4386662.
51. Brady S W, Liu Y, Ma X, Gout A M, Hagiwara K, Zhou X, et al. Pan-neuroblastoma analysis reveals age- and signature-associated driver alterations. Nature communications. 2020; 11(1):5183. Epub 2020/10/16. doi: 10.1038/s41467-020-18987-4. PubMed PMID: 33056981; PubMed Central PMCID: PMCPMC7560655.
52 Brodeur G M, Bagatell R. Mechanisms of neuroblastoma regression. Nat Rev Clin Oncol. 2014; 11(12):704-13. Epub 2014/10/22. doi: 10.1038/nrclinonc.2014.168. PubMed PMID: 25331179; PubMed Central PMCID: PMCPMC4244231.
53. Morandi F, Sabatini F, Podesta M, Airoldi I. Immunotherapeutic Strategies for Neuroblastoma: Present, Past and Future. Vaccines (Basel). 2021; 9(1). Epub 2021/01/17. doi: 10.3390/vaccines9010043. PubMed PMID: 33450862; PubMed Central PMCID: PMCPMC7828327.
54. Caruso D A, Orme L M, Amor G M, Neale A M, Radcliff F J, Downie P, et al. Results of a Phase I study utilizing monocyte-derived dendritic cells pulsed with tumor RNA in children with Stage 4 neuroblastoma. Cancer. 2005; 103(6):1280-91. Epub 2005/02/05. doi: 10.1002/cncr.20911. PubMed PMID: 15693021.
55. Kabir T F, Kunos C A, Villano J L, Chauhan A. Immunotherapy for Medulloblastoma: Current Perspectives. Immunotargets Ther. 2020; 9:57-77. Epub 2020/05/06. doi: 10.2147/ITT.S198162. PubMed PMID: 32368525; PubMed Central PMCID: PMCPMC7182450.
56 Kauer M, Ban J, Kofler R, Walker B, Davis S, Meltzer P, et al. A molecular function map of Ewing's sarcoma. PloS one. 2009; 4(4):e5415. Epub 2009/05/01. doi: 10.1371/journal.pone.0005415. PubMed PMID: 19404404; PubMed Central PMCID: PMCPMC2671847.
57 Sankar S, Lessnick S L. Promiscuous partnerships in Ewing's sarcoma. Cancer Genet. 2011; 204(7):351-65. Epub 2011/08/30. doi: 10.1016/j.cancergen.2011.07.008. PubMed PMID: 21872822; PubMed Central PMCID: PMCPMC3164520.
58. Morales E, Olson M, Iglesias F, Dahiya S, Luetkens T, Atanackovic D. Role of immunotherapy in Ewing sarcoma. J Immunother Cancer. 2020; 8(2). Epub 2020/12/10. doi: 10.1136/jitc-2020-000653. PubMed PMID: 33293354; PubMed Central PMCID: PMCPMC7725096.
59. Peng W, Huang X, Yang D. EWS/FLI-1 peptide-pulsed dendritic cells induces the antitumor immunity in a murine Ewing's sarcoma cell model. International immunopharmacology. 2014; 21(2):336-41. Epub 2014/05/28. doi: 10.1016/j.intimp.2014.05.013. PubMed PMID: 24861249.
60. Richters M M, Xia H, Campbell K M, Gillanders W E, Griffith O L, Griffith M. Best practices for bioinformatic characterization of neoantigens for clinical utility. Genome Med. 2019; 11(1):56. Epub 2019/08/30. doi: 10.1186/s13073-019-0666-2. PubMed PMID: 31462330; PubMed Central PMCID: PMCPMC6714459.
61. Szolek A, Schubert B, Mohr C, Sturm M, Feldhahn M, Kohlbacher O. OptiType: precision HLA typing from next-generation sequencing data. Bioinformatics. 2014; 30(23):3310-6. Epub 2014/08/22. doi: 10.1093/bioinformatics/btu548. PubMed PMID: 25143287; PubMed Central PMCID: PMCPMC4441069.
62. Larjo A, Eveleigh R, Kilpelainen E, Kwan T, Pastinen T, Koskela S, et al. Accuracy of Programs for the Determination of Human Leukocyte Antigen Alleles from Next-Generation Sequencing Data. Frontiers in immunology. 2017; 8:1815. Epub 2018/01/13. doi: 10.3389/fimmu.2017.01815. PubMed PMID: 29326702; PubMed Central PMCID: PMCPMC5733459.
63. Jones D T, Kocialkowski S, Liu L, Pearson D M, Backlund L M, Ichimura K, et al. Tandem duplication producing a novel oncogenic BRAF fusion gene defines the majority of pilocytic astrocytomas. Cancer Res. 2008; 68(21):8673-7. Epub 2008/11/01. doi: 10.1158/0008-5472.CAN-08-2097. PubMed PMID: 18974108; PubMed Central PMCID: PMCPMC2577184.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

1. A method of treating a subject affected by cancer by providing tumor-mutation specific T cells from an MHC allele-matched donor, comprising the following steps:
obtaining a biopsy of the subject's tumor;
obtaining sequences for proteins in the biopsy;
identifying proteins from the biopsy containing mutated amino acids and the peptide comprising each of the mutated amino acids;
determining T cell exposed motifs which comprise mutated amino acids in each of the proteins;
determining the predicted binding affinity to the subject's MHC alleles of peptides which comprise each of the T cell exposed motifs comprising mutated amino acids, or a subset thereof;
generating an array of alternative peptides not present in the tumor, wherein each peptide in the array comprises the amino acids of one of the T cell exposed motifs, and in which the amino acids not within the T cell exposed motif are substituted to change the predicted MHC binding affinity;
selecting a group of one or more selected peptides from the array of alternative peptides which have a desired predicted binding affinity for one or more of the subject's MHC alleles; and
synthesizing the group of one or more selected peptides, or nucleic acids encoding the selected peptides; and
obtaining T cells from a donor who carries at least one matched MHC allele to the subject; and
contacting dendritic cells in vitro with the selected peptides, or nucleic acids encoding the selected peptide, then contacting the dendritic cells with the T cells from the donor; and
multiplying in vitro the T cells responsive to the selected peptide; and infusing the T cells responsive to the selected peptide from the donor into the subject.

2. The method of claim 1 wherein the subject's MHC alleles are MHC I alleles.

3. The method of claim 1 wherein the subject's MHC alleles are MHC II alleles.

4. The method of claim 1, wherein the T cell exposed motifs comprise 5 sequential amino acids that engage the T cell receptor of a CD8+ T cell.

5. The method of claim 1, wherein the T cell exposed motifs comprise a discontinuous sequence of 5 amino acids that engage the T cell receptor of a CD4+ T cell.

6-9. (canceled)

10. The method of claim 1, wherein the donor carries one or more MHC I alleles matched to those of the subject.

11. The method of claim 1, wherein the donor carries one or more MHC II alleles matched to those of the subject.

12. (canceled)

13. The method of claim 1 wherein the donor and subject are matched in at least 25% of their MHC alleles.

14-22. (canceled)

23. The method of claim 1, wherein the T cells responsive to the selected peptide are preferentially separated from non-responsive T cells prior to infusion to the subject.

24-25. (canceled)

26. The method of claim 1, wherein the donor is vaccinated with the selected peptides of claim 1 prior to donation of cells.

27-30. (canceled)

31. The method of claim 1, wherein the infusion of the subject with the T cells responsive to the selected peptides is followed by vaccination of the subject with one or more of the selected peptides or nucleic acids encoding the peptides.

32. The method of claim 1, wherein a tissue sample from the donor is sequenced to determine if the tumor mutations found in the subject are hereditary.

33. The method of claim 1, further comprising analyzing the selected peptides to reduce the potential of adverse off-target binding in the subject, the analysis comprising:
searching a reference database of T cell exposed motifs in the human proteome to identify T cell exposed motifs that match the T cell exposed motifs which comprise mutated amino acids in the subject;
determining the predicted MHC binding of the peptides in the human proteome which comprise the T cell exposed motifs;
determining if there is high predicted binding to the subject's MHC alleles;
evaluating if the peptides in the human proteome occur in proteins which constitute a risk of adverse reactions if targeted by a T cell; and
eliminating any selected peptide comprising T cell exposed motifs giving rise to the risk.

34-36. (canceled)

37. The method of claim 1, wherein the subject is under 25 years of age.

38. (canceled)

39. The method of claim 1, wherein the tumor is a solid tumor.

40. The method of claim 1, wherein the tumor is a pediatric tumor.

41. The method of claim 40 wherein the pediatric tumor is selected from the group consisting of medulloblastoma, neuroblastoma, rhabdomyosarcoma, Ewing's sarcoma, osteosarcoma and Wilms' tumor.

42-49. (canceled)

50. The method of claim 26, further comprising analyzing the selected peptides to reduce the potential of adverse off-target binding in the donor, the analysis comprising:
searching a reference database of T cell exposed motifs in the human proteome to identify T cell exposed motifs that match the T cell exposed motifs which comprise mutated amino acids in the selected peptides;
determining the predicted MHC binding of the peptides in the human proteome which comprise the T cell exposed motifs;
determining if there is high predicted binding to the donor's MHC alleles;
evaluating if the peptides in the human proteome occur in proteins which constitute a risk of adverse reactions if targeted by a T cell; and
eliminating any selected peptide comprising T cell exposed motifs giving rise to the risk.
